(19) United States
(12) Patent Application Publication
(10) Pub. No.: US 2025/0085287 A1
(43) Pub. Date: Mar. 13, 2025

(54) METHODS FOR DETECTION OF MEMBRANE BOUND GLYPICAN-3

(71) Applicant: ADICET THERAPEUTICS, INC., Redwood City, CA (US)

(72) Inventors: Sandra M. HAYES, San Mateo, CA (US); Jonathan Ta Shin WONG, San Francisco, CA (US); Arun BHAT, Seattle, WA (US); Mustafa TURKOZ, Redwood City, CA (US); Erika MEADDOUGH, Gilory, CA (US); Hui SHAO, San Mateo, CA (US); Jacqueline Kennedy WILDE, Sunnyvale, CA (US); Ori MALLER, Half Moon Bay, CA (US); Elizabeth PEREZ, Mountain View, CA (US); Matthew Ian HOOPES, Mount Shasta, CA (US); Kevin NISHIMOTO, San Mateo, CA (US)

(21) Appl. No.: 18/684,672

(22) PCT Filed: Aug. 19, 2022
(86) PCT No.: PCT/US2022/040931

§ 371 Date: Feb. 17, 2024

Related U.S. Application Data
(60) Provisional application No. 63/235,093, filed on Aug. 19, 2021.

(51) Int. Cl. G01N 33/574; A61K 47/68; C07K 14/725; C07K 16/30; G01N 33/577

(52) U.S. Cl. CPC G01N 33/57492; A61K 47/6859; C07K 14/7051; C07K 16/303; G01N 33/57469; G01N 33/577; C07K 2317/24; C07K 2317/31; C07K 2317/565; C07K 2317/92; G01N 2800/52

(57) ABSTRACT

Embodiments provide for anti-GPC3 antibodies compositions comprising the same, and methods of using such antibodies and compositions for the prevention, diagnosis, and treatment of cancer. In one embodiment, a method for predicting a therapeutic effect of an anti-GPC3 immunotherapy on a cancer characterized in that cells of the cancer express GPC3, comprises detecting the presence of said cells in a subject via an immunohistochemical methodology, and wherein when the presence of said cells is detected, the anti-GPC3 immunotherapy is predicted to have a therapeutic effect on the cancer in the subject.

### CROSS-REFERENCE TO RELATED APPLICATIONS

This application claims the benefit of priority to U.S. Provisional Application No. 63/235,093 filed on Aug. 19, 2021.

### FIELD OF THE INVENTION

The present invention relates to antibodies that target cancer cells expressing glypican-3 on their cell surface, and to compositions and methods of using such antibodies for the prevention, diagnosis, and treatment of such cancers.

### BACKGROUND

Glypican-3 (GPC3) is a membrane-bound heparin sulfate proteoglycan that is overexpressed in approximately 70%-80% of hepatocellular carcinomas (HCCs), as well as yolk sac tumors, gastric carcinoma, colorectal carcinoma, non-small cell lung cancer, and thyroid cancer (Moek et al., 2018. The American Journal of Pathology, 8:9; 1973-1981), yet is largely unexpressed in common healthy tissues. In this context, GPC3 represents a promising tumor antigen target. However, GPC3 can be expressed in the cytoplasm as well as on the membrane. Because certain promising treatment therapies (e.g., chimeric antigen receptor T-cell (CAR-T) therapy) are only currently capable of recognizing GPC3 on the cell surface, there is a need for therapies that specifically target cell surface GPC3, and for diagnostic methodologies capable to accurately assess GPC3 levels on the surface of tumor cells.

To date, there is just one commercially available GPC3 immunohistochemistry (IHC) in-vitro diagnostic (IVD) assay, which is a qualitative assay using an anti-GPC3 mAb (1G12) specific to the C-terminus of GPC3 (Cell Marque™, Rocklin, CA). According to the specifications of the assay, the GPC3 antibody displays a diffuse and membranous staining pattern in the neoplastic cells of HCCs. Furthermore, it has been noted that the sensitivity of the 1G12 mAb is low in tumor cell lines with low expression levels (Phung et al., 2012, mAbs Landes Bioscience, 4:5; 592-599). Thus, based on at least the above, there is a need for mAbs with higher sensitivity and that are capable of preferentially staining cell membranes of GPC3-expressing tumor cells, particularly for use in IVD assays for assessing membrane-bound GPC3.

### SUMMARY OF THE INVENTION

The present invention addresses and resolves the foregoing shortcomings in the prior art with compositions and methods providing improved discrimination between membrane-bound and cytosolic GPC3, for more accurate diagnostic analyses and treatments. In some embodiments, the invention provides anti-GPC3 antibodies, including fragments thereof, and methods of using the same, e.g., for the diagnosis, prevention, and/or treatment of cancer.

In one embodiment, anti-GPC3 antibodies of the invention bind to a GPC3 epitope that is positioned in a C-terminal beta chain of GPC3. In one embodiment, the anti-GPC3 antibody comprises a heavy chain variable region comprising SEQ ID NO: 2 and a light chain variable region comprising SEQ ID NO: 4.

In one embodiment, the heavy chain of an anti-GPC3 antibody of the present invention comprises a complementary determining region (CDR) 1 set forth as SEQ ID NO: 6, a CDR2 set forth as SEQ ID NO: 8, and a CDR3 set forth as SEQ ID NO: 10.

In one embodiment, the light chain of an anti-GPC3 antibody of the present invention comprises a CDR1 set forth as SEQ ID NO: 13, a CDR2 set forth as SEQ ID NO: 15, and a CDR3 set forth as SEQ ID NO: 17.

In one embodiment, the heavy chain of an anti-GPC3 antibody of the present invention comprises a complementary determining region (CDR) 1 set forth as SEQ ID NO: 6, a CDR2 set forth as SEQ ID NO: 8, and a CDR3 set forth as SEQ ID NO: 10, and the light chain of the an anti-GPC3 antibody of the present invention comprises a CDR1 set forth as SEQ ID NO: 13, a CDR2 set forth as SEQ ID NO: 15, and a CDR3 set forth as SEQ ID NO: 17.

In one embodiment, the heavy chain of an anti-GPC3 antibody comprises a CDR1, a CDR2, and a CDR3, respectively set forth as amino acid residues 31-35, 50-66, and 99-105 of SEQ ID NO: 2, and the light chain of the antibody comprises a CDR1, a CDR2, and a CDR3 respectively set forth as amino acid residues 24-34, 50-56, and 89-97 of SEQ ID NO: 4.

In one embodiment, the invention provides an anti-GPC3 antibody that competes with an antibody comprising a heavy chain variable region comprising SEQ ID NO:2 and a light chain variable region comprising SEQ ID NO: 4 for binding to GPC3.

Anti-GPC3 antibodies of the invention include, for example, monoclonal antibodies, antibody fragments, including Fab, Fab′, F(ab′)2, and Fv fragments, diabodies, single domain antibodies, chimeric antibodies, humanized antibodies, single-chain antibodies and antibodies that competitively inhibit the binding of an antibody comprising a heavy chain variable region comprising SEQ ID NO:2 and a light chain variable region comprising SEQ ID NO:4 to the GPC3.

In one embodiment, an anti-GPC3 antibody comprises a heavy chain variable region comprising an amino acid sequence selected from the group consisting of SEQ ID NO: 6, SEQ ID NO: 8, and SEQ ID NO: 10.

In one embodiment, an anti-GPC3 antibody comprises a light chain variable region comprising an amino acid sequence selected from the group consisting of SEQ ID NO: 13, SEQ ID NO: 15, and SEQ ID NO: 17.

In one embodiment, the anti-GPC3 antibody is a chimeric, humanized, or human antibody.

In one embodiment, the anti-GPC3 antibody is a monoclonal antibody.

In one embodiment, the anti-GPC3 antibody is an antibody fragment.

In one embodiment, the anti-GPC3 antibody is a bispecific antibody.

In one aspect, the invention provides a method for diagnosing cancer in a subject, comprising detecting the presence of GPC3 on cell surface of cells comprising the cancer in the subject or in a biological sample from the subject.

In one aspect, the invention provides a method for determining the prognosis for a subject diagnosed with cancer, comprising detecting the presence of GPC3 expressed on a cell surface of cells comprising the cancer in the subject or in a biological sample obtained from the subject. In one embodiment, the method involves detecting the presence of GPC3 in the subject or in a biological sample from the subject after the subject has received a therapeutic agent for the treatment of cancer. In embodiments, the therapeutic agent is an agent for treatment of a cancer that comprises cells of the cancer that express GPC3 on their cell surface.

In one aspect, the invention provides a method for predicting a therapeutic effect of an anti-GPC3 immunotherapy on a cancer. In embodiments, the cancer is comprised of cells that express GPC3. In embodiments, the method comprises detecting the presence of the cells, wherein when the cells are detected, the anti-GPC3 immunotherapy is predicted to have a therapeutic effect on the cancer in the subject. In embodiments, the predicting is conducted prior to the subject having received any anti-GPC3 immunotherapy. In embodiments, the predicting is conducted while the subject is in the process of receiving anti-GPC3 immunotherapy.

In one aspect, the invention provides nucleic acids encoding a GPC3 antibody (or portion(s) thereof) of the invention.

In one aspect, the invention provides vectors comprising DNA encoding any of the herein described anti-GPC3 antibodies or portions thereof. Host cells comprising any such vector are also provided. By way of example, the host cells may be CHO cells, E. coli cells, or yeast cells. A process for producing any of the herein described polypeptides is further provided and comprises culturing host cells under conditions suitable for expression of the desired polypeptide and recovering the desired polypeptide from the cell culture. In one embodiment, the vectors comprise SEQ ID NO: 1 and/or SEQ ID NO: 3 (Table 2).

In one aspect, the invention provides a CAR modified immune cell, preferably a CAR-T or CAR-NK cell, comprising a chimeric antigen receptor capable of binding to GPC3, preferably capable of binding to the beta chain of GPC3. In one aspect, the invention provides a CAR modified immune cell, preferably a CAR-T or CAR-NK cell, comprising a chimeric antigen receptor, wherein the chimeric antigen receptor comprises a light chain variable region of an anti-GPC3 antibody of the present disclosure, and a heavy chain variable region of an anti-GPC3 antibody of the present disclosure.

In one aspect, the invention provides a CAR modified immune cell (or plurality thereof), preferably a CAR-T or CAR-NK cell, comprising an anti-GPC3 antibody. In one embodiment, the anti-GPC3 antibody is an antibody fragment. In one embodiment, the anti-GPC3 antibody is an scFv. In one embodiment, the modified T-cell is an αβ T cell. In one embodiment, the modified T-cell is a γδ T cell.

In one aspect, the invention provides a pharmaceutical composition, comprising an anti-GPC3 antibody and a pharmaceutically acceptable carrier. In one aspect, the invention provides a pharmaceutical composition, comprising a CAR modified immune cell, preferably a CAR-T or CAR-NK cell, of the invention, and a pharmaceutically acceptable carrier. In one embodiment, the anti-GPC3 antibody is used in the form of an antibody-drug conjugate (ADC).

In one aspect, the invention provides methods for making an anti-GPC3 antibody. In one aspect, the invention provides methods for making a CAR modified immune cell disclosed herein. In one embodiment, the invention provides methods for making an ADC comprising an anti-GPC3 antibody.

In one aspect, the invention provides a method for the preparation of a medicament for the treatment of cancer. In embodiments, the invention is directed to the use of an anti-GPC3 antibody as disclosed herein, for the preparation of a medicament useful in the treatment of a condition which is responsive to the anti-GPC3 antibody.

In one aspect, the invention provides use of a nucleic acid of the invention in the preparation of a medicament for the therapeutic and/or prophylactic treatment of a disease, such as a cancer, a tumor and/or a cell proliferative disorder.

In one aspect, the invention provides use of an expression vector of the invention in the preparation of a medicament for the therapeutic and/or prophylactic treatment of a disease, such as a cancer, a tumor and/or a cell proliferative disorder.

In one aspect, the invention provides use of a host cell of the invention in the preparation of a medicament for the therapeutic and/or prophylactic treatment of a disease, such as a cancer, a tumor and/or a cell proliferative disorder.

In one aspect, the invention provides ADCs comprising an anti-GPC3 antibody conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In another aspect, the invention further provides methods of using the immunoconjugates. In one aspect, an immunoconjugate comprises any of the above anti-GPC3 antibodies covalently attached to a cytotoxic agent or a detectable agent.

In one aspect, the invention provides a method of inhibiting the proliferation or growth of a cell that expresses GPC3 on its cell surface, comprising contacting the cell with an anti-GPC3 antibody or CAR modified immune cell, preferably a CAR-T or CAR-NK cell, of the invention. In one embodiment, the anti-GPC3 antibody is used in the form of an ADC. In embodiments, the proliferation or growth of the cell comprises a cell proliferative disorder. In embodiments, the cell proliferative disorder is cancer.

In one aspect, the invention provides a method of therapeutically treating a mammal having a cancerous tumor comprising a cell that expresses GPC3, said method comprising administering to said mammal a therapeutically effective amount of an antibody or CAR modified immune cell(s), preferably a CAR-T or CAR-NK cell(s) of the invention, thereby effectively treating said mammal. In one embodiment, the mammal is a human subject. In one embodiment, the cancer is selected from the group consisting of liver cancer, ovarian cancer, lung cancer, Merkel cell carcinoma, and gastric or stomach cancer

In one aspect, the invention provides a method of inducing death of a cell that expresses GPC3 on its cell surface, comprising contacting the cell with an anti-GPC3 antibody or CAR modified immune cell(s), preferably a CAR-T or CAR-NK cell(s), of the invention. In one embodiment, the anti-GPC3 antibody is an ADC.

In a still further aspect, the invention concerns a composition of matter comprising an anti-GPC3 antibody as described herein, in some embodiments in combination with a carrier. Optionally, the carrier is a pharmaceutically acceptable carrier. In a still further aspect, the invention concerns a composition of matter comprising CAR modified immune cells, preferably a CAR-T or CAR-NK cells as described herein, in combination with a carrier. Optionally, the carrier is a pharmaceutically acceptable carrier.

Also provided herein are kits and methods of using the same.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DESCRIPTION OF THE FIGURES

Embodiments are illustrated by way of example and not by way of limitation in the figures of the accompanying drawings.

FIGS. 1A-1C illustrate results of biolayer inferometry (BLI) binding assays conducted using 204 (FIG. 1A), 1G12 (FIG. 1B), and GC33 (FIG. 1C).

FIG. 2A depicts detection of recombinant human (rh) GPC3 by 204 (left panels), GC33 (middle panels), and 1G12 (right panels), under reducing (R) and non-reducing (NR) conditions by western blot.

FIG. 2B depicts western blotting results of the 204 and 1G12 antibodies used to probe rhGPC3, rhGPC5, and rhGPC6 under reducing (R) and non-reducing (NR) conditions.

FIG. 3 depicts western blot analysis of soluble native human GPC3 detected by 204. Samples tested were obtained as supernatants from tumor cell lines HepG2, NCI-H661, and Hep3B.

FIG. 4 is a high-level schematic illustration of a major GPC3 isoform (isoform 2), illustrating the alpha chain, beta chain, furin cleavage site, GC33 immunogen, 1G12 immunogen, GC33 epitope, and possible 204 epitope.

FIG. 5 is another high-level schematic illustration of GPC3, showing an ADAM10 cleavage site, and region of 204 binding as compared to GC33.

FIG. 6A depicts a coomassie-stained gel showing cleavage of rhGPC3 with ADAM10 and ADAM17. An approximately 12 kDa fragment is liverated by cleavage of GPC3 with ADAM10.

FIG. 6B depicts western blot analysis of ADAM10 and ADAM17-cleaved GPC3 as probed with 204 and GC33. The approximately 12 kDa fragment mentioned with regard to FIG. 6A is detected by GC33 but not 204, indicating that the epitope for 204 is between the furin-cleavage site and the predicted ADAM10 site.

FIG. 7 illustrates a high-level example optimized immunohistochemistry (IHC) method for use with the 204 antibody of the present disclosure.

FIG. 8 depicts representative images from a tumor microarray (TMA) from a human hepatocellular carcinoma (HCC) using the 204 antibody and the optimized protocol of FIG. 7. A semi-quantitative membrane-associated H-score was used to evaluate staining, as shown.

FIG. 9 depicts representative images of IHC experiments using 204 or 1G12 to detect GPC3 in squamous cell carcinoma of the lung, and HCC, along with corresponding membrane-associated H-scores, using the optimized protocol of FIG. 7.

FIG. 10 depicts representative images of IHC experiments using 204 or 1G12 to probe healthy lung and liver tissue, using the optimized protocol of FIG. 7.

FIG. 11 depicts IHC images of tissues from HepG2 (GPC3^{hi}) and PP5 (GPC3^{lo}) tumors stained with 1G12 (0.5 μg/mL) or 204 (0.1 μg/mL) antibodies, and visualized via 3,3′-diaminobenzidine (DAB) as a substrate for secondary antibody-conjugated horseradish peroxidase (HRP). Also shown are isotype controls. The human hepatocellular carcinoma (HCC) cell lines HepG2 (GPC3^{hi}) and PP5 (GPC3^{lo}) were implanted subcutaneously in NOD SCID mice, and tumors were harvested on day 24, and day 31 post-implantation for PP5 and HepG2, respectively.

FIG. 12 shows bar graphs quantifying the IHC staining corresponding to the images of FIG. 11. Quantified is a HepG2 tumor, and two different PP5 tumors. The top panel of bar graphs depict membrane-associated H-score, and the bottom panel of bar graphs refers to total H-score (cytoplasmic and membrane).

FIG. 13 depicts plots showing head-to-head comparison of membrane-associated H-scores obtained using 204 and 1G12 in IHC experiments on formalin fixed paraffin embedded (FFPE) tumor blocks (top graph) and FFPE tumor cores from tissue microarrays (TMAs) (bottom graph) for various cancers including gastric cancer (adenocarcinoma), liver cancer (HCC), lung cancer (squamous cell carcinoma), and ovarian cancer (clear cell carcinoma).

FIGS. 14A-14C show prevalence distribution of membrane-associated GPC3 in HCC and SCCL (FIG. 14A), HCC (FIG. 14B), and SCCL (FIG. 14C) based on staining intensities using 204 and 1G12 mAbs for IHC.

FIGS. 14D-14E show prevalence distribution of membrane-associated GPC3 in HCC and SCCL (FIG. 14D), HCC (FIG. 14E), and SCCL (FIG. 14F) based on membrane-associated H-scores using 204 and 1G12 mAbs for IHC.

FIG. 15 depicts images of membrane-associated GPC3 expression in FFPE tissues from xenograft tumor models using 204 mAb as compared to 1G12 mAb. Cell lines used for the xenograft procedure include Hep3B, HepG2, Huh-7, and PLC/PRF/5. Insets (larger square) in each image correspond to higher resolution images of the denoted region (smaller square). Shown for reference are the corresponding membrane-associated H-scores for each condition.

FIG. 16A-16B depict graphs quantifying the IHC experiments of FIG. 15, in terms of membrane-associated H-score (FIG. 16A), and % of moderate-to-high membrane intensity (FIG. 16B).

FIG. 17A is a table showing scoring of tumors from xenograft mouse models as measured based on IHC using the 204 mAb.

FIG. 17B is a table showing scoring of tumors from xenograft mouse models as measured based on IHC using the 1G12 mAb.

### DETAILED DESCRIPTION OF THE DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense.

Various operations may be described as multiple discrete operations in turn, in a manner that may be helpful in understanding embodiments; however, the order of description should not be construed to imply that these operations are order-dependent.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims. Any embodiments or features of embodiments can be combined with one another, and such combinations are expressly encompassed within the scope of the present invention.

Unless defined otherwise, all technical and scientific terms used herein have the same meaning as commonly understood by one of ordinary skill in the art to which this invention belongs.

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

One skilled in the art will recognize many methods and materials similar or equivalent to those described herein, which could be used in the practice of the present invention. Indeed, the present invention is in no way limited to the methods and materials described.

Although any methods and materials similar or equivalent to those described herein can be used in the practice or testing of the present invention, the preferred methods and materials are now described. All patents, applications and non-patent publications mentioned in this specification are incorporated herein by reference in their entireties.

### I. DEFINITIONS

For purposes of interpreting this specification, the following definitions will apply, and whenever appropriate, terms used in the singular will also include the plural and vice versa. In the event that any definition set forth conflicts with any document incorporated herein by reference, the definition set forth below shall control. Unless defined otherwise, all technical and scientific terms used herein have the same meaning as commonly understood by one of ordinary skill in the art to which the invention pertains.

“About” as used herein when referring to a measurable value such as an amount, a temporal duration, and the like, is meant to encompass variations of ±20% or ±10%. more preferably ±5%, even more preferably ±1%, and still more preferably ±0.1% from the specified value, as such variations are appropriate to perform the disclosed methods. Furthermore, recitation of a range of numerical values includes any numerical value encompassed by said range, and/or any range of values included within said range. For example, a numerical range of 1-10 encompasses the range, and additionally encompasses individual numerical values (e.g., 1, 2, 3, 4, 5, 6, 7, 8, 9, 10), and ranges within said numerical range (e.g., 1-2, 1-4, 2-5, 3-7, 4-9, 5-10, and so on).

“Contacting,” as used herein, includes bringing together at least two substances in solution or solid phase.

“Glypican-3 (GPC3)” as used herein, refers to a member of the glypican family of heparan sulfate (HS) proteoglycans that are attached to the cell surface by a glycosylphosphatidylinositol anchor (Filmus and Selleck, J Clin Invest 108:497-501, 2001). The GPC3 gene codes for a core protein of approximately 70 kD, which can be cleaved by furin to produce an N-terminal 40 kD fragment and a C-terminal 30 kD fragment. Two HS chains are attached on the C-terminal portion of GPC3. GPC3 and other glypican family proteins play a role in cell division and cell growth regulation. GPC3 is highly expressed in HCC and some other human cancers including melanoma, squamous cell carcinomas of the lung, and clear cell carcinomas of the ovary (Ho and Kim, Eur J Cancer 47 (3): 333-338, 2011), but is not expressed in normal tissues. GPC3 is also known as SGB, DGSX, MXR7, SDYS, SGBS, OCI-5, SGBS1 and GTR2-2.

There are four known isoforms of human GPC3 (isoforms 1-4). Nucleic acid and amino acid sequences of the four isoforms of GPC3 are known, including GenBank Accession numbers: NM_001164617 and NP_001158089 (isoform 1); NM_004484 and NP_004475 (isoform 2); NM_001164618 and NP_001158090 (isoform 3); and NM_001164619 and NP_001158091 (isoform 4). In some embodiments of the present disclosure, the antibodies disclosed herein bind one or more of the four human GPC3 isoforms, or a conservative variant thereof.

A “modification” of an amino acid residue/position, as used herein, refers to a change of a primary amino acid sequence as compared to a starting amino acid sequence, wherein the change results from a sequence alteration involving said amino acid residue/positions. For example, typical modifications include substitution of the residue (or at said position) with another amino acid (e.g., a conservative or non-conservative substitution), insertion of one or more (generally fewer than 5 or 3) amino acids adjacent to said residue/position, and deletion of said residue/position. An “amino acid substitution”, or variation thereof, refers to the replacement of an existing amino acid residue in a predetermined (starting) amino acid sequence with a different amino acid residue. Generally and preferably, the modification results in alteration in at least one physicobiochemical activity of the variant polypeptide compared to a polypeptide comprising the starting (or “wild type”) amino acid sequence. For example, in the case of an antibody, a physicobiochemical activity that is altered can be binding affinity, binding capability and/or binding effect upon a target molecule.

As used herein, the term “T lymphocyte” or “T cell” refers to an immune cell that expresses or has expressed CD3 (CD3+) and a T Cell Receptor (TCR+). T cells play a central role in cell-mediated immunity. A T cell that “has expressed CD3 and a TCR” has been engineered to eliminate CD3 and/or TCR cell surface expression.

As used herein, the term “TCR” or “T cell receptor” refers to a dimeric heterologous cell surface signaling protein forming an alpha-beta or gamma-delta receptor or combinations thereof. αβTCRs recognize an antigen presented by an MHC molecule, whereas γδTCR can recognize an antigen independently of MHC presentation.

The term “MHC’ (major histocompatibility complex) refers to a subset of genes that encodes cell-surface antigen-presenting proteins in humans, these genes are referred to as human leukocyte antigen (HLA) genes. Herein, the abbreviations MHC or HLA are used interchangeably.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies (including agonist, antagonist, neutralizing antibodies, full length or intact monoclonal antibodies), antibody compositions with polyepitopic specificity, multivalent antibodies, multispecific antibodies (e.g., bispecific antibodies so long as they exhibit the desired biological activity), formed from at least two intact antibodies, diabodies, single domain antibodies (sdAbs), as long as they exhibit the desired biological or immunological activity, Fv, Fab and F(ab), as well as single chain antibodies and humanized antibodies (Harlow et ah, 1999, In: Using Antibodies: A Laboratory Manual, Cold Spring Harbor Laboratory Press, NY: Harlow et ah, 1989, In; Antibodies: A Laboratory Manual, Cold Spring Harbor, N.Y.; Houston et ah, 1988, Proc. Nat Acad. Sci. USA 85:5879-5883: Bird et ah, 1988, Science 242:423-426).

“Antibody fragments” comprise a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870, Example 2; Zapata et al., Protein Eng. 8 (10): 1057-1062 [1995]); single-chain antibody molecules; disulfide linked fragment variable (dsFv), and multispecific antibodies formed from antibody fragments. In one embodiment, an antibody fragment comprises an antigen binding site of the intact antibody and thus retains the ability to bind antigen. Also included among antibody fragments are portions of antibodies (and combinations of portions of antibodies, for example, scFv) that may be used as targeting arms, directed for example to a GPC3 tumor epitope, in chimeric antigenic receptors of CAR-T cells or CAR-NK cells. Such fragments are not necessarily proeteolytic fragments but rather portions of polypeptide sequences that can confer affinity for target.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CHI). Each Fab fragment is monovalent with respect to antigen binding, i.e., it has a single antigen-binding site. Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra. In one embodiment, an anti-GPC3 antibody derived scFv is used as the targeting arm of a CAR-T cell or a CAR-NK cell disclosed herein.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The term “hypervariable region”, “HVR”, or “HV”, when used herein refers to the regions of an antibody variable domain which are hypervariable in sequence and/or form structurally defined loops. Generally, antibodies comprise six hypervariable regions; three in the VH (HI, H2, H3), and three in the VL (LI, L2, L3). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, MD. (1991)). Chothia refers instead to the location of the structural loops (Chothia and Lesk J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35 A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these hypervariable regions are noted below.

### Loop Kabat AbM Chothia Contact

### LI L24-L34 L24-L34 L24-L34 L30-L36

### L2 L50-L56 L50-L56 L50-L56 L46-L55

### L3 L89-L97 L89-L97 L89-L97 L89-L96

### HI H31-H35B H26-H35B H26-H32 . . . 34 H30-H35B

### (Kabat Numbering)

### HI H31-H35 H26-H35 H26-H32 H30-H35

### (Chothia Numbering)

### H2 H50-H65 H50-H58 H52-H56 H47-H58

### H3 H95-H102 H95-H102 H95-H102 H93-H101

Hypervariable regions may comprise “extended hypervariable regions” as follows: 24-36 or 24-34 (LI), 46-56 or 50-56 (L2) and 89-97 (L3) in the VL and 26-35B (HI), 50-65, 47-65 or 49-65 (H2) and 93-102, 94-102 or 95-102 (H3) in the VH. The variable domain residues are numbered according to Kabat et al., supra for each of these definitions.

“Framework” or “FR” residues are those variable domain residues other than the hypervariable region residues herein defined.

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, MD. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgGI EU antibody. Unless stated otherwise herein, references to residue numbers in the variable domain of antibodies means residue numbering by the Kabat numbering system.

A “blocking” antibody or an “antagonist” antibody is one which inhibits or reduces biological activity of the antigen it binds. Preferred blocking antibodies or antagonist antibodies substantially or completely inhibit the biological activity of the antigen.

An antibody that “binds” an antigen or epitope of interest is one that binds the antigen or epitope with sufficient affinity that is measurably different from a non-specific interaction. Specific binding can be measured, for example, by determining binding of a molecule compared to binding of a control molecule, which generally is a molecule of similar structure that does not have binding activity.

An antibody that inhibits the growth of tumor cells is one that results in measurable growth inhibition of cancer cells. In one embodiment, an anti-GPC3 antibody is capable of inhibiting the growth of cancer cells displaying a GPC3 tumor epitope. Preferred growth inhibitory anti-GPC3 antibodies inhibit growth of GPC3-expressing tumor cells by greater than 20%, preferably from about 20% to about 50%, and even more preferably, by greater than 50% (e.g., from about 50% to about 100%) as compared to the appropriate control, the control typically being tumor cells not treated with the antibody being tested (or treated with isotype controls).

Anti-GPC3 antibodies may (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind.

The term “antagonist” is used in the broadest sense, and includes any molecule that partially or fully blocks, inhibits, or neutralizes a biological activity of antigen. Suitable antagonist molecules specifically include antagonist antibodies or antibody fragments, fragments or amino acid sequence variants of native GPC3 polypeptides, peptides, antisense oligonucleotides, small organic molecules, etc. Methods for identifying antagonists of a GPC3 polypeptide, may comprise contacting a GPC3 polypeptide with a candidate antagonist molecule, and measuring a detectable change in one or more biological activities normally associated with the GPC3 polypeptide.

The terms “anti-GPC3 antibody”, “GPC3 antibody”, and “an antibody that binds to GPC3” are used interchangeably. Anti-GPC3 antibodies are preferably capable of binding GPC3 with sufficient affinity such that the antibody is useful as a diagnostic and/or therapeutic agent.

In one embodiment, anti-GPC3 antibody is used herein to specifically refer to an anti-GPC3 monoclonal antibody that (i) comprises the heavy chain variable domain of SEQ ID NO: 2 and/or the light chain variable domain of SEQ ID NO: 4 as shown in Table 2; or (ii) comprises one, two, three, four, five, or six of the CDRs shown in Table 1.

An “isolated antibody” is one which has been identified and separated and/or recovered from a component of its natural environment. Contaminant components of its natural environment are materials which would interfere with therapeutic uses for the antibody, and may include enzymes, hormones, and other proteinaceous or nonproteinaceous solutes.

The basic 4-chain antibody unit is a heterotetrameric glycoprotein composed of two identical light (L) chains and two identical heavy (H) chains. In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CHI). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, CT, 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in CH sequence and function, e.g., humans express the following subclasses: IgGI, IgG2, IgG3, IgG4, IgA1, and IgA2. The “variable region” or “variable domain” of an antibody refers to the amino-terminal domains of the heavy or light chain of the antibody. The variable domain of the heavy chain may be referred to as “VH” or “VH” The variable domain of the light chain may be referred to as “VL” or “¼.”. These domains are generally the most variable parts of an antibody and contain the antigen-binding sites.

The term “variable” refers to the fact that certain segments of the variable domains differ extensively in sequence among antibodies. The V domain mediates antigen binding and defines specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)).

An “intact” antibody is one which comprises an antigen-binding site as well as a CL and at least heavy chain constant domains, CHI, CH2 and CH3. The constant domains may be native sequence constant domains (e.g. human native sequence constant domains) or amino acid sequence variant thereof. Preferably, the intact antibody has one or more effector functions.

By the term “synthetic antibody,” as used herein, is meant an antibody which is generated using recombinant DNA technology. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

A “chimeric antibody” has framework residues from one species, such as human, and CDRs (which generally confer antigen binding) from another species, such as a murine antibody that specifically binds GPC3.

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

The term “binding” in the context of binding of an antibody, Ig, antibody-binding fragment, to either an antigen or other molecule (e.g., sugar), typically refers to an interaction or association between a minimum of two entities, or molecular structures, such as an antibody-antigen interaction.

“Conservative” amino acid substitutions are those substitutions that do not substantially affect or decrease the affinity of a protein, such as an antibody to GPC3. For example, a monoclonal antibody that specifically binds GPC3 can include at most about 1, at most about 2, at most about 5, and most about 10, or at most about 15 conservative substitutions and specifically bind the GPC3 polypeptide. The term “conservative variant” also includes the use of a substituted amino acid in place of an unsubstituted parent amino acid, provided that antibody specifically binds GPC3. Non-conservative substitutions are those that reduce an activity or binding to GPC3.

Conservative amino acid substitution tables providing functionally similar amino acids are well known to one of ordinary skill in the art. The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

The term “antigen” or “Ag” as used herein is defined as a molecule that provokes an immune response. This immune response may involve either antibody production, or the activation of specific immunologically-competent cells, or both. The skilled artisan will understand that any macromolecule, including proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated, synthesized, or can be derived from a biological sample. Such a biological sample can include, but is not limited to a tissue sample, a tumor sample, a cell or a biological fluid.

The term “epitope” includes any protein determinant, lipid or carbohydrate determinant capable of specific binding to an immunoglobulin or T-cell receptor (e.g., a specific antigen binding site). Epitopic determinants usually consist of active surface groupings of molecules such as amino acids, lipids or sugar side chains and usually have specific three-dimensional structural characteristics, as well as specific charge characteristics. An antibody is said to specifically bind an antigen when the equilibrium dissociation constant (Kd) is in a range of Oct. 6, 2010-12. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain.

The term “chimeric antigen receptors (CARs),” as used herein, may refer to artificial T-cell receptors, T-bodies, single-chain immunoreceptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy in specific embodiments, CARs direct specificity of the cell to a tumor associated antigen, for example. In some embodiments, CARs comprise an intracellular activation domain (allowing the T cell to activate upon engagement of targeting moiety with target ceil, such as a target tumor cell), a transmembrane domain, and an extracellular domain that may vary in length and comprises a disease- or disorder-associated, e.g., a tumor-antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3C, FcR, CD27, CD28, CD137, DAP 10/12 and/or OX40, 4-1BB, 1COS, TLRs (e.g., TLR2) etc. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, an cytokine receptors. Furthermore, one skilled in the art will understand that a costimuliatory domain need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response.

The term “immunoconjugate” or “antibody drug conjugate” (ADC) refers to covalent linkage of an effector molecule to an antibody or functional fragment thereof. The effector molecule can be a detectable label or an immunotoxin. Specific, non-limiting examples of toxins include, but are not limited to, abrin, ricin, Pseudomonas exotoxin (PE, such as PE35, PE37, PE38, and PE40), diphtheria toxin (DT), botulinum toxin, or modified toxins thereof, or other toxic agents that directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as the domain Ia of PE and the B chain of DT) and replacing it with a different targeting moiety, such as an antibody.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “therapeutically effective amount” refers to the amount of a composition that will elicit a biological or medical response of a tissue, system, or subject that is being sought by the researcher, veterinarian, medical doctor or other clinician. The term “therapeutically effective amount” includes that amount of a composition that, when administered, is sufficient to prevent development of, or alleviate to some extent, one or more of the signs or symptoms of the disorder or disease (e.g., solid tumor) being treated. The therapeutically effective amount will vary depending on the composition, the disease and its severity and the age, weight, etc., of the subject to be treated.

To “treat” a disease as the term is use herein, means to reduce the frequency or severity of at least one sign or symptom of a disease or disorder experienced by a subject.

Administration “in combination with” one or more further therapeutic agents includes simultaneous (concurrent) and sequential administration in any order.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

“Encoding” refers to the inherent property of specific sequences of nucleotides in a polynucleotide, such as a gene, a cDNA, or an inRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence an is usually provided in sequence listings, and the non-coding strand, use as the template for transcription of a gene or cDNA, can be referred to as encoding the protein or other product of that gene or cDNA.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form (e.g., monoclonal antibody of the present disclosure), or can exist in a non-native environment such as, for example, a host cell.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The terms “patient,” “subject,” “individual,” and the like are used interchangeably herein, and refer to any animal, amenable to the methods described herein. In certain non-limiting embodiments, the patient, subject or individual is a human.

By the term “specifically binds,” or “specifically recognizes” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

In some embodiments, specific binding can be characterized by an equilibrium dissociation constant of at least about I×10⁻⁸ M or less (e.g., a smaller value denotes a tighter binding). Methods for determining whether two molecules specifically bind are well known in the art and include, for example, equilibrium dialysis, surface plasmon resonance, and the like.

The term “K_{D}” (M), as used herein, refers to the dissociation equilibrium constant of a particular binding protein-ligand interaction. For example, K_{D} may refer to the dissociation equilibrium constant between an antibody, Ig, or antibody-binding fragment and an antigen. There is an inverse relationship between K_{D} and binding affinity, therefore the smaller the K_{D} value, the higher, i.e., stronger, the affinity. Thus, the terms “higher affinity” or “stronger affinity” relate to a higher ability to form an interaction and therefore a smaller K_{D} value, and conversely the terms “lower affinity” or “weaker affinity” relate to a lower ability to form an interaction and therefore a larger K_{D} value. The dissociation equilibrium constant K_{D} is equal to 1/K.

The term “kₐ” (M⁻¹×sec⁻¹), as used herein, refers to the association rate constant of a particular protein-antigen (e.g., antibody-antigen) interaction.

The term “k_{d}” (sec⁻¹), as used herein, refers to the dissociation rate constant of a particular protein-antigen interaction (e.g., antibody-antigen).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), skin cancer, melanoma, lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer (e.g., pancreatic ductal adenocarcinoma), glioblastoma, cervical cancer, ovarian cancer (e.g., high grade serous ovarian carcinoma, ovarian clear cell carcinoma), liver cancer (e.g., hepatocellular carcinoma (HCC)), bladder cancer (e.g., urothelial bladder cancer), testicular (germ cell tumour) cancer, hepatoma, breast cancer, brain cancer (e.g., astrocytoma), colon cancer, rectal cancer, colorectal cancer, endometrial or uterine carcinoma, salivary gland carcinoma, kidney or renal cancer (e.g., renal cell carcinoma, nephroblastoma or Wilms' tumour), prostate cancer, vulval cancer, thyroid cancer, hepatic carcinoma, anal carcinoma, penile carcinoma, as well as head and neck cancer. Additional examples of cancer include, without limitation, retinoblastoma, thecomas, arrhenoblastomas, hepatoma, hematologic malignancies including non-Hodgkins lymphoma (NHL), multiple myeloma and acute hematologic malignancies, endometrial or uterine carcinoma, endometriosis, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, and urinary tract carcinomas.

In a preferred embodiment, the cancer is liver cancer (e.g., HCC). In another preferred embodiment, the cancer is gastric or stomach cancer (e.g., adenocarcinoma). In another preferred embodiment, the cancer is lung cancer (e.g., squamous cell carcinoma). In another preferred embodiment, the cancer is ovarian cancer (e.g., ovarian clear cell carcinoma). The term “metastatic cancer” means the state of cancer where the cancer cells of a tissue of origin are transmitted from the original site to one or more sites elsewhere in the body, by the blood vessels or lymphatics, to form one or more secondary tumors in one or more organs besides the tissue of origin. A prominent example is metastatic breast cancer.

As used herein, a “GPC3-associated cancer” is a cancer that is associated with overexpression of a GPC3 gene or gene product and/or is associated with display of a GPC3 tumor epitope. Suitable control cells can be, for example, cells from an individual who is not affected with cancer or non-cancerous cells from the subject who has cancer.

The present methods include methods of treating a subject having cancer. Particularly cancer that is associated with expression of GPC3. The present methods also include methods for modulating certain cell behaviours, particularly cancer cell behaviours, particularly cancer cells GPC3 on their cell surface.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues.

The terms “predictive” and “prognostic” as used herein are also interchangeable. In one sense, the methods for prediction or prognostication are to allow the person practicing a predictive/prognostic method of the invention to select patients that are deemed (usually in advance of treatment, but not necessarily) more likely to respond to treatment with an anticancer agent, preferably an anti-GPC3 antibody or a CAR-T cell or CAR-NK cell of the invention.

“Solid tumors” as referred to herein are tumors that comprise a tumor mass of at least about 10 or at least about 100 tumor cells. The solid tumor can be a soft tissue tumor, a primary solid tumor, or a metastatic lesion. Examples of solid tumors relevant to the present disclosure include but are not limited to, e.g., sarcomas, adenocarcinomas, and carcinomas, of the various organ systems, such as those affecting liver, lung, gastrointestinal (e.g., colon), genitourinary tract (e.g., renal, urothelial cells), and the like. Adenocarcinomas include malignancies such as most colon cancers, rectal cancer, renal-cell carcinoma, liver cancer, non-small cell carcinoma of the lung, cancer of the small intestine and cancer of the esophagus. Metastatic lesions of the aforementioned cancers can also be treated or prevented using the methods and compositions of the invention.

In some embodiments, the solid tumor cell expresses, or over-expresses, glypican3 (GPC3). In some embodiments, the solid tumor cell expresses, or over-expresses an epitope of GPC3 that is specifically bound by an anti-GPC3 antibody, T cell Receptor, or chimeric antigen receptor described herein (e.g., 204 monoclonal antibody) and/or in U.S. Pat. No. 7,919,086; WO 2014/180306; WO 2018/019772; WO 2016/049459; WO 2003/000883; WO 2006/046751; WO 2007/047291; WO 2016/086813; WO 2016/047722; WO 2016/036973; WO 2020/072546; Cancer Res. 2008; 68:9832-9838: Proc Natl Acad Sci USA. 2013 Mar. 19; 110 (12): E 1083-1, the contents of each of which are incorporated by reference in the entirety and for all purposes and in particular for the binding domains, antibodies, antibody fragments, complementarity determining regions, polypeptides containing said complementarity determining regions, nucleic acids encoding for said complementarity determining regions, and epitope specificities and assays for determining epitope specificity described therein. In some embodiments, the solid tumor cell expresses, or over-expresses an epitope of GPC3 that is specifically bound by the anti-GPC3 antibody GC33, or 1G12, or 204. In some embodiments, the solid tumor expresses, or over-expresses, an HLA: peptide complex containing a GPC3 fragment. In some embodiments, the HLA is a class I HLA, such as HLA-A2.

### II. COMPOSITIONS AND METHODS OF THE INVENTION

### A. Overview of Anti-GPC3 Antibodies

In one aspect, the invention provides anti-GPC3 antibodies, including fragments thereof, compositions comprising the same, and methods of using the same for various purposes, including the treatment of cancer. In one aspect, the invention provides an antibody that binds to a beta chain of GPC3 expressed on the surface of cells (e.g., tumor cells). Optionally, the antibody is a monoclonal antibody, antibody fragment, including Fab, Fab′, F(ab′)2, and scFv fragment, diabody, single domain antibody, chimeric antibody, humanized antibody, single-chain antibody or antibody that competitively inhibits the binding of an anti-GPC3 antibody to its respective antigenic epitope. The antibodies of the present invention may optionally be produced in CHO cells or bacterial cells or by other means. For detection purposes, the anti-GPC3 antibodies of the present invention may be detectably labeled, attached to a solid support, or the like.

In one aspect, an antibody that binds to GPC3 is provided, wherein the antibody comprises a heavy chain variable region comprising:

(SEQ ID NO: 2)
EVQLQQSGPELVKPGASVKISCKTSGYTFTEYAMHWVKQSHGKSLEWIG
GINPNNGVTTYNQRFKGKATLTVDKSSSTAYMELRSLTSEDSAVYYCAR
GLLWYAYWGQGTLVTVSA

In one aspect, an antibody that binds to GPC3 is provided, wherein the antibody comprises a light chain variable region comprising:

(SEQ ID NO: 4)
DIKMTQSPSSMYASLGERVTITCKASQDINSYLSWFQQKPGKSPKTLIY
RANRLVDGVPSRFSGSGSGQDYSLTISSLEYEDMGIYYCLQYDEFPLTF
GAGTKLELK

In one aspect, an antibody that binds to GPC3 is provided, wherein the antibody comprises a heavy chain variable region comprising SEQ ID NO:2 and a light chain variable region comprising SEQ ID NO:4.

In one aspect, an antibody that binds to GPC3 is provided, wherein the antibody comprises a heavy chain variable region comprising a CDR1 comprising an amino acid sequence set forth as EYAMH (SEQ ID NO: 6).

In one aspect, an antibody that binds to GPC3 is provided, wherein the antibody comprises a heavy chain variable region comprising a CDR2 comprising an amino acid sequence set forth as GINPNNGVTTYNQRFKG (SEQ ID NO: 8).

In one aspect, an antibody that binds to GPC3 is provided, wherein the antibody comprises a heavy chain variable region comprising a CDR3 comprising an amino acid sequence set forth as GLLWYAY (SEQ ID NO: 10).

In one aspect, an antibody that binds to GPC3 is provided, wherein the antibody comprises a light chain variable region comprising a CDR1 comprising an amino acid sequence set forth as KASQDINSYLS (SEQ ID NO: 13).

In one aspect, an antibody that binds to GPC3 is provided, wherein the antibody comprises a light chain variable region comprising a CDR2 comprising an amino acid sequence set forth as RANRLVD (SEQ ID NO: 15).

In one aspect, an antibody that binds to GPC3 is provided, wherein the antibody comprises a light chain variable region comprising a CDR3 comprising an amino acid sequence set forth as LQYDEFPLT (SEQ ID NO: 17).

In one aspect, an antibody that binds to GPC3 is provided, wherein the antibody comprises a heavy chain variable region comprising a CDR1 set forth as SEQ ID NO: 6; a CDR2 set forth as SEQ ID NO: 8; and a CDR3 set forth as SEQ ID NO: 10.

In one aspect, an antibody that binds to GPC3 is provided, wherein the antibody comprises a light chain variable region comprising a CDR1 set forth as SEQ ID NO: 13; a CDR2 set forth as SEQ ID NO: 15; and a CDR3 set forth as SEQ ID NO: 17.

In one aspect, an antibody that binds to GPC3 is provided, wherein the antibody comprises a heavy chain variable region comprising a CDR1 set forth as SEQ ID NO: 6; a CDR2 set forth as SEQ ID NO: 8; and a CDR3 set forth as SEQ ID NO: 10; and further comprises a light chain variable region comprising a CDR1 set forth as SEQ ID NO: 13; a CDR2 set forth as SEQ ID NO: 15; and a CDR3 set forth as SEQ ID NO: 17. In one embodiment, an antibody of the invention comprising these sequences (in combination as described herein) is a humanized or human antibody.

In one aspect, the invention includes an anti-GPC3 antibody comprising (i) a heavy chain variable domain comprising SEQ ID NO: 2; and/or (ii) a light chain variable domain comprising SEQ ID NO: 4.

In some embodiments, these antibodies further comprise a human subgroup III heavy chain framework consensus sequence. In one embodiments of these antibodies, these antibodies further comprise a human κï light chain framework consensus sequence.

In one aspect, an anti-GPC3 antibody competes for binding to a tumor displayed GPC3 (for example, as displayed on HCC cells) with an anti-GPC3 antibody comprising a heavy chain variable region comprising SEQ ID NO: 2 and a light chain variable region comprising SEQ ID NO 4.

A more comprehensive description of anti-GPC3 antibodies encompassed by the present disclosure is presented below.

### B. Methods of Detection

An embodiment of the present invention is directed to a method of determining the presence of a GPC3 polypeptide in a sample suspected of containing the GPC3 polypeptide, wherein the method comprises exposing the sample to an antibody that binds to the GPC3 polypeptide and determining binding of the antibody to the GPC3 polypeptide in the sample, wherein the presence of such binding is indicative of the presence of the GPC3 polypeptide in the sample. Optionally, the sample may contain cells (which may be cancer cells) suspected of expressing the GPC3 polypeptide. The antibody employed in the method may optionally be detectably labeled, attached to a solid support, or the like.

The binding of the anti-glypican 3 antibody to glypican 3 can be detected preferably by immunohistochemistry (IHC) methodology as herein disclosed, but it may be understood that said binding is not limited to IHC methodology, but can comprise a method generally known by those skilled in the art. For example, ELISA (enzyme-linked immunosorbent assay), EIA (enzyme immunoassay), RIA (radioimmunoassay), immunofluorescence, western blotting, and the like can be used. Relevant methods are described in the general textbook “Antibodies A Laboratory Manual. Ed Harlow, David Lane, Cold Spring Harbor Laboratory, 1988”.

### Exemplary IHC Assay

As discussed herein, there is a need for diagnostic methodologies capable of accurately assessing GPC3 levels on the surface of tumor cells. This is at least because certain immunotherapies (e.g., CAR-T therapy) rely on the interaction between cell-surface GPC3 expressed on tumor cells, and a corresponding anti-GPC3 antibody or antigen-binding fragment. In this context, in vitro IHC methodology relying on anti-GPC3 monoclonal antibodies is herein disclosed that presents advantages over the use of other prior art antibodies. Specifically, the IHC methodology herein disclosed was arrived at by requiring the assay to display a number of advantageous criteria. First, the disclosed IHC IVD assay was constrained to exhibit the substantial absence of non-specific background. Non-specific background in the context of an IHC IVD can complicate analysis and scoring of the staining of tissue samples, and hence can lead to inaccurate assessments of the prevalence (or lack thereof) of a detected target (e.g., membrane-bound GPC3 in the context of this disclosure). Accordingly, as disclosed herein and as exemplified in the Examples, the disclosed IHC IVD assay exhibits a substantial absence of non-specific background signal. Second, the assay was constrained to exhibit distinct linear demarcation at the cell surface. This advantageously enables high confidence scoring of the membrane-bound GPC3 staining. Third, the assay was constrained to exhibit clear and unambiguous nuclear counterstain. Fourth, the assay was constrained to rely on an antibody (204 as herein disclosed) that enabled the assay to meet the above-mentioned criteria, while also exhibiting high accuracy, sensitivity, and specificity, as well as a low nanomolar affinity for GPC3 as disclosed and exemplified herein.

a. Tissue Preparation

The term “tissue preparation” used herein refers to a biological preparation obtained from individuals, body fluids (e.g., blood, serum, plasma, spinal fluid), tissue cultures, tissue sections, or the like. Preferably, the tissue preparation is a subject-derived preparation, for example tissue obtained from a tumor of the subject. Biopsy, a method known in the art, is preferably used as a method of collecting said tissue. In examples, the tissue preparation is a liver tissue, or a lung tissue, or a gastric tissue, or an ovarian tissue, however other sources of tissue are within the scope of this disclosure, including any tissue that harbors GPC-3-expressing tumor cell(s). As an exemplary illustration, a biopsy may be used to collect a liver tissue by the direct insertion of a thin long needle into a subject's liver from the skin surface. The site of the puncture with the needed may be between ribs in the lower right chest, although other sites are within the scope of this disclosure. The procedure includes confirming the safety of the puncture site, for example via reliance on an ultrasonic examination apparatus, followed by disinfection of the puncture site, anesthetization of the region from the skin to the liver surface, and finally the puncturing via use of a puncture needed following a small incision of the skin at the puncture site. Although not specifically described, similar biopsy methodology may be used to collect tissue from other bodily locations (e.g., lung, gastrointestinal system, ovary, and the like), and such methodology will be readily understood to the skilled person.

Tissue preparations of the present disclosure are observed with a transmitted light under a microscope, hence are cut into thin slices to facilitate light used in the microscope to sufficiently penetrate said preparations. Prior to cutting into thin slices, the tissue preparations are fixed. Briefly, the tissue preparations to be fixed are cut using a cutting tool (e.g., surgical knife) into fragments having a size and a shape suitable for preparing paraffin-embedded sections. Subsequently, the fragments are dipped in a fixative, a reagent used for carrying out fixation. The fixative used is preferably formalin, more preferably neutral buffered formalin. The concentration of the neutral buffered formalin is appropriately selected according to the characteristics or physical properties of the tissue preparations. The concentration can be changed appropriately between 1 and 50%, preferably between 5 and 25%, more preferably between 10 and 15%, for use. The fixative containing the tissue preparations dipped therein is appropriately degassed using a vacuum pump. The fixation is carried out by leaving the tissue preparations in the fixative for several hours under conditions involving normal pressure and room temperature. The time required for the fixation can be selected appropriately within the range of 1 hour to 7 days, preferably 2 hours to 3 days, more preferably 3 hours to 24 hours, even more preferably 4 hours to 16 hours. The preparations thus fixed are further appropriately dipped in a phosphate buffer or the like for several hours (the time can be selected appropriately within the range of 2 hours to 48 hours, preferably 3 hours to 24 hours, more preferably 4 hours to 16 hours).

Next, from the fixed tissue preparations, sections can be prepared preferably using frozen section method or paraffin section method. Preferable examples of the frozen section method include a method which involves freezing the tissues by addition into O.C.T. compound (Miles. Inc.) and cutting the frozen tissues into thin slices using a cryostat (frozen section preparing apparatus). In the paraffin section method, the fixed tissue preparations are dipped in an embedding agent, which is then solidified to thereby impart uniform and appropriate hardness to the sections. Paraffin can be used preferably as the embedding agent. The fixed tissue preparations are dehydrated using ethanol, or a combination of ethanol washes and xylene washes. In one example, the tissue preparations are dehydrated by sequentially dipping the tissue preparations in 70% ethanol, 80% ethanol, and 100% ethanol. The time required for the dipping and the number of dips can be selected appropriately within the ranges of 1 min to 1 hour to several days and 1 time to 3 times. Moreover, the dipping may be performed at room temperature or at 4° C. For the dipping at 4° C., a longer dipping time (e.g., overnight) is preferable. In another example, tissue preparations are dehydrated by sequential dipping in 95% EtOH, 100% EtOH, Again, the time required for each dipping procedure and the number of dips can be selected appropriately within the ranges of 1 min to 1 hour to several days and 1 time to 3 times. Subsequently, the liquid phase is replaced by xylene, and then, the tissue preparations are embedded in paraffin. The time required for the replacement of the liquid phase by xylene can be selected appropriately within the range of several minutes to several hours. In this procedure, the replacement may be performed at room temperature or at 4° C. For the replacement at 4° C., a longer replacement time (e.g., overnight) is preferable. The time required for the paraffin embedding and the number thereof can be selected appropriately within the ranges of 1 hour to several hours and 1 time to 4 times. In this procedure, the embedding may be performed at room temperature or at 4° C. For the embedding at 4° C., a longer embedding time (e.g., overnight) is preferable. Moreover, the tissue preparations can be paraffin-embedded preferably by use of a paraffin embedding apparatus (e.g., EG1160, Leica Microsystems) which automatically processes paraffin embedding reaction.

The tissue preparations thus paraffin-embedded are bonded to a scaffold to prepare a “block”, which is then cut using a microtome into thin slices of the desired thickness selected from thicknesses of 1 to 20 μm. The thin tissue sections thus cut are left standing on slide glass as a transparent support for bonding. In this case, slide glass that is coated with 0.01% poly-L-lysine (Sigma-Aldrich Co.) for preventing peel-off of the tissue sections and dried can also be used preferably. The bonded tissue sections are dried in air for an appropriate time selected from between several minutes and 1 hour.

In some additional or alternative examples, the present disclosure employs the use of tissue microarrays (TMAs). In an example, TMAs are constructed by removing a core (e.g., tube-shaped section) of tissue from paraffin block (donor block, FFPE tissue) using a hollow needle, and transferring this core to a predetermined position on a single paraffin block (e.g., recipient block). TMAs can be used to compare control and test samples (e.g., positive control, negative control, test samples from various tissue regions or types) within one slide constructed on a semi-automated platform. As an example, individual arrays can be constructed with as many as 360 cores of 0.6 mm diameter, or 187 cores of 1 mm diameter, or 60 cores of 2 mm diameter, etc. Such an example is meant to be illustrative and non-limiting.

b. Antigen Retrieval

In an exemplary method of the present invention, the reactivity of an antigen whose reactivity has been reduced due to formalin fixation is retrieved. In one example, a protease-induced epitope retrieval method (PIER method) can be used. Briefly, the methodology includes digesting section with protease (e.g., trypsin, pepsin, or the like) prior to immunostaining. The protease used in the protease-induced epitope retrieval method is not particularly limited in its type or origin, and generally available protease can be selected appropriately for use. Preferable examples of the protease used include pepsin with a concentration of 0.05% in 0.01 N hydrochloric acid, trypsin with a concentration of 0.1% further containing 0.1% CaCl₂) in a Tris buffer (pH 7.6), and protease K with a concentration of 1 to 50 μg/ml in a 10 mM Tris-HCl buffer (pH 7.8) containing 10 mM EDTA and 0.5% SDS.

Furthermore, when the protease K is used, the pH of its reaction solution is appropriately selected from between 6.5 and 9.5 and SH reagent or a trypsin or chymotrypsin inhibitor may be used appropriately. Protease included in Histofine Her2 kit (MONO) (Nichirei Bioscience) is also included in such specific examples of preferable protease. The protease-induced epitope retrieval is usually performed at 37° C. However, the reaction temperature can be changed appropriately within the range of 25° C. to 50° C. When the protease-induced epitope retrieval is performed at 37° C., the reaction time is appropriately selected from between, for example, 1 minute and 5 hours and is, for example, 15 minutes, 30 minutes, 45 minutes, 1 hour, 2 hours, 3 hours, or 4 hours. After the completion of the PIER treatment, the tissue preparations thus treated are washed with a wash buffer. PBS (phosphate-buffered saline) is preferably used as the wash buffer. In addition, a Tris-HCl buffer can also be used preferably. The washing conditions usually adopt a method involving performing washing at room temperature for 5 minutes three times. However, the washing time and temperature can be changed appropriately.

In another exemplary method, the reactivity of an antigen whose reactivity has been reduced due to formalin fixation is retrieved via a heat-induced epitope retrieval method (HIER method). Specifically, heating using a microwave, boiling, or an autoclave allegedly enables an epitope to bind to antibodies as a result of hydrolyzing the antigen by the high-temperature treatment. When the boiling treatment is performed at an output of 780 W to keep the temperature of the solution at approximately 98° C., the time required for the retrieval including the treatment is appropriately selected from between 5 minutes and 60 minutes and is, for example, 10 minutes. The antigen retrieval treatment can be performed in a 10 mM sodium citrate buffer as well as commercially available Diva Decloaker solution (Biocare Medical, LLC, Pacheo, CA), BOND Epitope Retrieval Solution 1 (ER1) or BOND Epitope Retrieval Solution 2 (ER2) (Leica Biosystems Richmond, Inc, Richmond, IL), or the like. Any buffer or aqueous solution is preferably used as long as an epitope in the antigen recognized by an anti-glypican 3 antibody acquires affinity for the antibody as a result of retrieval treatment such that membrane-bound GPC3 can be detected by the 204 antibody of the present disclosure without appreciable non-specific background, and exhibiting linear demarcation of staining on the cell surface. Following completion of the retrieval treatment, the tissue preparations thus treated are left at room temperature for 30 minutes with gradual addition of DI water until slides are cooled.

c. Anti-Glypican 3 Antibody for Use in IHC IVD Assays

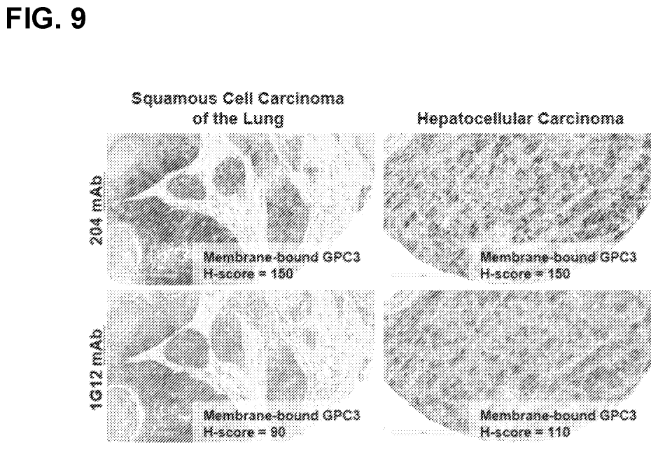

The preferred anti-glypican 3 antibody for use in the IHC IVD methodology of the present invention is 204 or a portion thereof. The 204 antibody as described in detail in the Examples is preferred as compared to, for example the GC33 antibody (WO 2006/006693) and 1G12 antibody (WO 2003/100429), as the 204 antibody was found to unexpectedly and advantageously exhibit lower non-specific background staining, linear demarcation of the cell surface, and to enable clear nuclear counterstain via hematoxylin. Accordingly, IHC staining using 204 was found to frequently result in higher membrane-associated H-scores as compared to 1G12 staining on the tissue samples (see, e.g., FIG. 9). This demonstrates that 204 as herein disclosed comprises an anti-GPC3 monoclonal antibody with higher sensitivity than 1G12 antibody and which is capable of preferentially staining cell membranes of GPC3-expressing cells, which as described above is an art-recognized problem in need of a solution (see e.g., Phung et al., 2012. mAbs Landes Bioscience, 4:5; 592-599). As discussed in Example 1 below, the anti-glypican 3 antibody preferably used in the present invention was obtained by immunizing non-human animals with glypican 3 as an immunizing antigen. General methods for preparing such anti-glypican 3 antibodies are described below in the Examples and in WO 2003/100429 and WO 2006/006693.

In embodiments, the preferred antibody comprises a heavy chain of the antibody that comprises a complementary determining region (CDR) 1 set forth herein as SEQ ID NO: 6, a CDR2 set forth herein as SEQ ID NO: 8, a CDR3 set forth herein as SEQ ID NO: 10, and the light chain of the antibody comprises a CDR1 set forth herein as SEQ ID NO: 13, a CDR2 set forth herein as SEQ ID NO: 15, and a CDR3 set forth herein as SEQ ID NO: 17. In some embodiments, the preferred antibody comprises a heavy chain variable region (HCVR) set forth herein as SEQ ID NO: 2, and a light chain variable region (LCVR) set forth herein as SEQ ID NO: 4.

d. Reaction of Tissue Preparations with Anti-Glypican 3 Antibody

Tissue preparations optionally subjected to antigen retrieval treatment as discussed above are reacted with (i.e., contacted with) the anti-GPC3 antibody (e.g., 204) as a primary antibody. The reaction is carried out under conditions suitable for the anti-GPC3 antibody to specifically recognize an epitope in the antigen (e.g., GPC3), thereby forming an antigen-antibody complex.

The reaction is usually performed overnight at 4° C. or at 37° C. for 1 hour. However, the reaction conditions can be changed appropriately within a range appropriate for recognition of an epitope in the antigen by the antibody and formation of an antigen-antibody complex. For example, the reaction temperature can be changed within the range of 4° C. to 50° C., and the reaction time can be changed between 1 minute and 7 days. For the reaction at low temperatures, a longer reaction time is preferable. After the completion of the primary antibody reaction, the tissue preparations are washed with a wash buffer. PBS (phosphate-buffered saline) is preferably used as the wash buffer. In addition, a Tris-HCl buffer can also be used preferably. The washing conditions usually adopt a method involving performing washing at room temperature for 5 minutes three times. However, the washing time and temperature can be changed appropriately.

Subsequently, the tissue preparations subjected to the primary antibody reaction are reacted with a secondary antibody recognizing the primary antibody. A secondary antibody labeled in advance with a labeling material for visualizing the secondary antibody is usually used. Preferable examples of the labeling material include: fluorescent dyes such as FITC (fluorescein isothiocyanate), Cy2 (Amersham Biosciences), and Alexa488 (Molecular Probes, Inc.); enzymes such as peroxidase and alkaline phosphatase; and colloidal gold.

The reaction with the secondary antibody is carried out under conditions appropriate for formation of an antigen-antibody complex by the anti-GPC3 antibody and the secondary antibody recognizing the anti-GPC3 antibody. The reaction is usually performed at room temperature or 37° C. for 30 minutes to 1 hour. However, the reaction conditions can be changed appropriately within a range appropriate for formation of an antigen-antibody complex by the anti-GPC3 antibody and the secondary antibody. For example, the reaction temperature can be changed within the range of 4° C. to 50° C., and the reaction time can be changed between 1 minute and 7 days. For the reaction at low temperatures, a longer reaction time is preferable. After the completion of the secondary antibody reaction, the tissue preparations are washed with a wash buffer. PBS (phosphate-buffered saline) is preferably used as the wash buffer. In addition, a Tris-HCl buffer can also be used preferably. The washing conditions usually adopt a method involving performing washing at room temperature for 5 minutes three times. However, the washing time and temperature can be changed appropriately.

Next, the tissue preparations subjected to the secondary antibody reaction are reacted with a substance for visualizing the labeling material. When peroxidase is used as the labeling material for the secondary antibody, the tissue preparations are incubated with a reaction solution obtained by mixing, immediately before the incubation, equal amounts of a 0.02% aqueous hydrogen peroxide solution and a DAB (diaminobenzidine) solution adjusted to a concentration of 0.1% with a 0.1 M Tris-HCl buffer (pH 7.2). In addition to DAB, chromogenic substrates such as DAB-Ni and AEC+ (Agilent Technologies, Santa Clara, CA), DAB sparkle (Biocare Medical, Pacheo, CA) can be selected appropriately. During the course of incubation, the degree of color development is observed under microscope at intervals. At the point in time when appropriate color development is confirmed, the visualization reaction is terminated by dipping the tissue preparations in PBS.

When alkaline phosphatase is used as the labeling material for the secondary antibody, the tissue preparations are incubated with a BCIP (5-bromo-4-chloro-3-indolyl phosphate)/NBT (nitro blue tetrazolium) (Zymed Laboratories Inc., San Francisco, CA) substrate solution (NBT at a concentration of 0.4 mM and BCIP at a concentration of 0.38 mM are dissolved in a 50 mM sodium carbonate buffer (pH 9.8) containing 10 mM MgCl₂ and 28 mM NaCl). Moreover, in addition to BCIP and NBT, Permanent Red, Fast Red, or Fuchsin+ (all Agilent) may be used appropriately. Prior to the incubation, the tissue preparations may be preincubated at room temperature for 1 minute to several hours with a 0.1 M Tris-HCl buffer (pH 9.5) containing levamisole chloride (inhibitor for endogenous alkaline phosphatase; Nacalai Tesque, Inc., Kyoto, Japan) at a concentration of 1 mM, 0.1 M sodium chloride, and 50 mM magnesium chloride. During the course of incubation, the tissue preparations are observed under microscope at intervals. At the point in time when the deposits of purple formazan, a final reaction product, are observed, the reaction is terminated by washing the tissue preparations with water or adding TBS containing 2% polyvinyl alcohol. Then, the tissue preparations are washed with TBST (TBS containing 0.1% Tween 20). When colloidal gold is used as the label for the secondary antibody, the colloidal gold is visualized by attaching metallic silver to the gold particles by silver enhancement. The silver enhancement method is generally known by those skilled in the art.

In embodiments, detection of the desired antibody-antigen complex can also be combined with nuclear staining. For example, nuclear staining can be done using hematoxylin, which stains nuclear components including heterochromatin and nucleoli. As a representative example, CAT Hematoxylin (Biocare Medical, Pacheo, CA) can be used for the histological demonstration of nuclear staining. Routinely used hematoxylin solutions are mordanted with aluminum, and typically used aluminum alum (ammonium aluminum sulfate) as the mordant salt. Because the aluminum salts are not in themselves oxidizers, it is necessary to expose the hematoxylin solution to air or chemical to affect the conversion of hematoxylin to hematein. The addition of acid to alum hematoxylin solutions (e.g., CAT Hematoxylin is thought to increase the selectivity of the stain for the nuclei and to counteract the rapid oxidizing effects of chemical oxidizing agents. This latter function enables the solution to maintain some hematoxylin in equilibrium with the hematein to ensure a better stain. Glycerol tends to stabilize the system against over-oxidation and aids in preventing rapid evaporation.

When any of fluorescent dyes such as FITC (fluorescein isothiocyanate), Cy2 (Amersham Biosciences, Amersham, UK), and Alexa488 (Molecular Probes, Inc., Eugene, OR) is used as the labeling material for the secondary antibody, the visualizing substance reaction step is unnecessary. A light emitted by irradiation with a light at the excitation wavelength of the fluorescent material can be detected appropriately by use of a fluorescence microscope.

In an exemplary embodiment, an in vitro immunoassay method for detecting the presence of GPC3-expressing cells in a subject comprises the steps of: a) providing a tissue preparation as a formalin-fixed paraffin embedded section from said subject, the formalin-fixed paraffin embedded section attached to a transparent support; (b) subjecting the tissue preparation to deparaffinization treatment; (c) optionally subjecting the tissue preparation to an antigen retrieval treatment; (d) contacting an anti-GPC3 antibody with the tissue preparation under conditions sufficient for formation of a complex of the anti-GPC3 antibody with GPC3 present on the cell membrane of cells of the tissue preparation treated in step (c); (e) detecting the presence of the complex by using immunohistochemistry, wherein when the complex is present, the subject is diagnosed as having a GPC3-expressing tumor; and wherein the anti-GPC3 antibody is a monoclonal antibody that specifically binds an epitope of a beta chain of GPC3, and where the heavy chain of the anti-GPC3 antibody comprises a complementary determining region (CDR) 1 set forth as SEQ ID NO: 6, a CDR2 set forth as SEQ ID NO: 8, and a CDR3 set forth as SEQ ID NO: 10, and the light chain of the antibody comprises a CDR1 set forth as SEQ ID NO: 13, a CDR2 set forth as SEQ ID NO: 15, and a CDR3 set forth as SEQ ID NO: 17. In examples, the GPC3 expressing tumor is selected from the group consisting of hepatocellular carcinoma, non-small cell lung cancer, ovarian clear cell carcinoma, and gastric cancer. In examples, the heavy chain of the anti-GPC3 antibody has a heavy chain variable region (HCVR) set forth as SEQ ID NO: 2. In examples, the light chain of the anti-GPC3 antibody has a light chain variable region (LCVR) set forth as SEQ ID NO: 4. In examples, the anti-GPC3 antibody is 204, wherein the 204 antibody specifically recognizes an epitope in the beta chain of GPC3 that is distinct from an epitope that is specifically recognized by 1G12 and that is additionally distinct from an epitope that is specifically recognized by GC33. In examples, the antigen retrieval treatment is based on a heat-induced epitope retrieval (HIER) method. In examples, the HIER method includes heating the tissue preparation of step (c) to between 105-115° C. for a timeframe between 10-20 minutes, preferably where the tissue preparation is headed to 110° C. for 15 minutes. In some examples, the antigen retrieval treatment is additionally or alternatively based on a protease-induced epitope retrieval (PIER) method. In examples, where the PIER method is used, the protease used the in the PIER method is selected from the group consisting of pepsin, trypsin, and protease K. In examples, detecting the presence of the complex by using immunohistochemistry comprises an enzymatic reaction. In examples, step (e) further comprises contacting the tissue preparation of step (d) with a secondary antibody conjugated to horseradish peroxidase (HRP) enzyme, and visualizing the complex via oxidation of 3,3′-diaminobenzidine by hydrogen peroxide in a reaction catalyzed by HRP. In examples, detecting the presence of the complex further comprises scoring the amount of the complex detected. In some examples, said scoring is done by a pathologist. In some examples, detecting the presence of the complex is done via digitization, and said scoring is automated based on the digitization of the detected complex. In some examples, said scoring further comprises determining a staining intensity of the complex detected via immunohistochemistry using an integer scale from 0 (negative) to 3+, recording the percentage of positively stained cells at each intensity level, and calculating a membrane-associated H-score based on the percentage of positively stained cells at each intensity level.

e. Automation of IHC IVD Assay

The IHC IVD assay as herein disclosed can be conducted manually, or can be automated. Relevant examples of automated systems for which the IHC IVD assay of the present disclosure can be carried out include but are not limited to Intellipath FLX® (Biocare Medical, Pacheo, CA), Autostainer Link 48 (Agilent Technologies, Santa Clara, CA), BOND-III fully automated IHC staining system (Leica Biosystems, Richmond, IL), and the like.

f. Classification of GPC3-Expressing Tissues and Prediction of Therapeutic Effect

It is known that GPC3 can release its N-terminal moiety into serum, for example upon digestion in cancerous tissues (e.g., liver cancer tissue) (WO 2004/022739). Thus, an antibody that reacts with the N-terminal portion of GPC3 would not be expected to be capable to bind to the C-terminal portion of the GPC3 polypeptide that remains anchored on the cell surface following digestion. As described below in the Examples, the 204 antibody is advantageous for use in the IHC IVD assay as herein disclosed due at least in part to it's ability to specifically recognize the C-terminal portion of GPC3 that remains anchored in the cell membrane following digestion in tumor tissues.

Anti-GPC3 antibodies are known to be useful in terms of the treatment and prevention of liver cancer (see for example WO 2004/022739), and there is evidence of anti-tumor activity imparted by cells expressing a CAR construct that specifically binds an epitope within GPC3 expressed on the surface of solid tumor cells (see for example WO 2020/072546). Because immunotherapy that relies on antibodies, CARs, and the like function by binding to cell surface GPC3, it is desirable that any prediction of therapeutic effect account primarily for membrane-associated GPC3 expression. In other words, when the therapeutic effect of a therapeutic anti-GPC immunotherapy (e.g., antibody, CAR, etc.) on GPC3-expressing tumor cells (e.g., solid tumor cells) is predicted depending on whether or not an epitope bound by an anti-GPC3 targeting agent is present in said GPC3-expressing cells, it is desirable that the methodology rely on an anti-GPC3 targeting agent that specifically binds the C-terminal portion of GPC3 that remains anchored in the cell membrane. As discussed herein and exemplified in the Examples, the 204 antibody, which is preferred in terms of use with the IHC IVD methodology of the present disclosure, specifically recognizes the C-terminal portion of GPC3 and preferentially binds to GPC3 expressed at the cell surface of tumor cells. Hence, the 204 antibody and its use thereof in the IHC IVD methodology herein disclosed is advantageous in that results from the assay are applicable to prediction of therapeutic effects of anti-GPC3 agents including but not limited to anti-GPC3 antibodies, cells expressing anti-GPC3 CAR(s), and the like.

The classification of GPC3-expressing cells/tissues relies on a scoring system that is based on one or more of staining intensity and membrane-associated H-score, but is not necessarily limited to said parameters. Briefly, staining intensity in the IHC IVD assay of the present disclosure is scored using a semi-quantitative integer scale from 0 (negative) to 3 (or “3+”). The percentage of positively staining cells at each intensity level is recorded. Scoring is preferably based on GPC3 localization to the cell membrane (apical and circumferential), but in some examples can also account for any cytoplasmic staining. H-scores are calculated as values between 0 and 300, defined as: 1×(percentage of cells staining at 1+intensity)+2×(percentage of cells staining at 2+intensity+3×(percentage of cells staining at 3+intensity)=H-score. The higher the H-score, the greater the indication of a predicted therapeutic effect of a therapeutic anti-GPC3 therapy (e.g., anti-GPC3 immunotherapy) on GPC3-expressing tumor cells. In some examples the scoring can be done by a certified pathologist. Additionally or alternatively, it is within the scope of this disclosure that the scoring can be automated. For example, the present disclosure provides for the digitizing of the difference in the degree and pattern of detection under a microscope of an antigen-antibody complex formed from GPC3 and anti-GPC3 antibody (e.g., 204). In examples where the scoring is done by a pathologist and/or where the scoring is digitized, test samples may be normalized to control samples, for example isotype control samples or similar tissue preparations lacking GPC3 expression on the cell surface.

g. Methods of Diagnosis and/or Treatment Based on IHC IVD Assays

The IHC IVD methodology of the present disclosure is useful in diagnosing a patient as having a cancer that comprises corresponding cells which express GPC3 on their cell membrane. The IHC IVD methodology of the present disclosure is additionally useful in determining whether to treat a patient with an anti-GPC3 therapy (e.g., antibody-based immunotherapy, CAR-based immunotherapy, and the like), or not, in a case where the patient has not already been receiving an anti-GPC3 therapy. Said IHC IVD methodology is also useful in determining whether to continue to treat a patient with an anti-GPC3 therapy under conditions where the patient has already been receiving said therapy for some amount of time. In some examples, dosage and/or dosing interval of an anti-GPC3 therapy may be adjusted (e.g., dosage may be increased or decreased, dosing interval may be increased or decreased, and the like) depending on the results of the IHC IVD assay as herein disclosed. Thus, the IHC IVD assay as herein disclosed can be used to diagnose a patient as having a particular cancer, i.e., a solid tumor that expressed GPC3 on the surface of cells comprising said tumor, and can also be used as a means of monitoring a patient's response to a cancer therapy, including but not limited to a cancer therapy that specifically targets GPC3 on the surface of the cancerous cells.

Thus, in one aspect, the invention provides a method of determining the presence of GPC3 in a sample suspected of containing GPC3, said method comprising exposing said sample to an antibody of the invention, and determining binding of said antibody to GP3 in said sample wherein binding of said antibody to GPC3 in said sample is indicative of the presence of said protein in said sample. In one embodiment, the sample is a biological sample (e.g., tissue preparation). In a further embodiment, the biological sample comprises liver cancer cells. In one embodiment, the biological sample is from a mammal experiencing or suspected of experiencing a liver cancer disorder and/or a liver cancer cell proliferative disorder. In a further embodiment, the biological sample comprises ovarian cancer cells. In one embodiment, the biological sample is from a mammal experiencing or suspected of experiencing an ovarian cancer disorder and/or an ovarian cancer cell proliferative disorder. In a further embodiment, the biological sample comprises gastric cancer (adenocarcinoma) cells. In one embodiment, the biological sample is from a mammal experiencing or suspected of experiencing a gastric or stomach disorder and/or a gastric or stomach cell proliferative disorder. In a further embodiment, the biological sample comprises lung cancer cells. In one embodiment, the biological sample is from a mammal experiencing or suspected of experiencing a squamous cell carcinoma disorder and/or a lung cancer cell proliferative disorder. In one embodiment, the biological sample comprises skin cells. In a further embodiment, the biological sample is from a mammal experiencing or suspected of experiencing Merkle cell carcinoma, or a melanoma.

In one aspect, a method of diagnosing a cell proliferative disorder associated with (i) an increase in cells, such as, e.g., liver cancer cells, ovarian cancer cells, lung cancer cells, or gastric or stomach cancer cells, expressing GPC3, or (ii) an increase in GPC3 expression within a tumor, is provided. In one embodiment, the method comprises contacting a test cell in a biological sample (e.g., tissue preparation) with an anti-GPC3 antibody of the present disclosure; determining the level of antibody bound to test cells in the sample by detecting binding of the antibody to GPC3; and comparing the level of antibody bound to cells in a control sample, wherein the level of antibody bound is normalized to the number of GPC3-expressing cells in the test and control samples, and wherein a higher level of antibody bound in the test sample as compared to the control sample indicates the presence of a cell proliferative disorder associated with cells expressing GPC3.

In one aspect, a method for predicting a therapeutic effect of an anti-GPC3 immunotherapy on a cancer is provided, the cancer characterized in that cells of the cancer express GPC3, the method comprising detecting the presence of said cells in a subject via the IVD IHC assay as herein disclosed. In embodiments, when the complex of the anti-GPC3 antibody with GPC3 expressed on the membrane of the cancer cells is detected, the anti-GPC3 immunotherapy is predicted to have a therapeutic effect on the cancer in the subject. In embodiments, the method of predicting the therapeutic effect is conducted prior to the subject having received any anti-GPC3 immunotherapy. In some embodiments, the method of predicting the therapeutic effect is conducted while the subject is already in the process of receiving the anti-GPC3 immunotherapy.

h. Related Detection Schemes and Assay Methodologies

Although a focus of the present invention is on IHC methodology using anti-GPC3 antibodies disclosed herein, it may be understood that the anti-GPC3 antibodies of the present invention may be employed in any known assay method, such as ELISA, competitive binding assays, direct and indirect sandwich assays, and immunoprecipitation assays (Zola, (1987) Monoclonal Antibodies: A Manual of Techniques, pp. 147-158, CRC Press, Inc.), and the like.

A detection label may be useful for localizing, visualizing, and quantitating a binding or recognition event. The labelled antibodies of the invention can detect cell-surface GPC3. Another use for detectably labelled antibodies is a method of bead-based immunocapture comprising conjugating a bead with a fluorescent labelled antibody and detecting a fluorescence signal upon binding of a ligand. Similar binding detection methodologies utilize the surface plasmon resonance (SPR) effect to measure and detect antibody-antigen interactions. Detection labels such as fluorescent dyes and chemiluminescent dyes (Briggs et al (1997) “Synthesis of Functionalised Fluorescent Dyes and Their Coupling to Amines and Amino Acids,” J. Chem. Soc, Perkin-Trans. 1:1051-1058) provide a detectable signal and are generally applicable for labelling antibodies, preferably with the following properties: (i) the labelled antibody should produce a very high signal with low background so that small quantities of antibodies can be sensitively detected in both cell-free and cell-based assays; and (ii) the labelled antibody should be photostable so that the fluorescent signal may be observed, monitored and recorded without significant photo bleaching. For applications involving cell surface binding of labelled antibody to membranes or cell surfaces, especially live cells, the labels preferably (iii) have good water-solubility to achieve effective conjugate concentration and detection sensitivity and (iv) are non-toxic to living cells so as not to disrupt the normal metabolic processes of the cells or cause premature cell death.

Direct quantification of cellular fluorescence intensity and enumeration of fluorescently labelled events, e.g. cell surface binding of peptide-dye conjugates may be conducted on an system (FMAT® 8100 HTS System, Applied Biosystems, Foster City, Calif.) that automates mix-and-read, non-radioactive assays with live cells or beads (Miraglia, “Homogeneous cell- and bead-based assays for high throughput screening using fluorometric microvolume assay technology”, (1999) J. of Biomolecular Screening 4:193-204). Uses of labelled antibodies also include cell surface receptor binding assays, inmmunocapture assays, fluorescence linked immunosorbent assays (FLISA), caspase-cleavage (Zheng, “Caspase-3 controls both cytoplasmic and nuclear events associated with Fas-mediated apoptosis in vivo”, (1998) Proc. Natl. Acad. Sci. USA 95:618-23; U.S. Pat. No. 6,372,907), apoptosis (Vermes, “A novel assay for apoptosis. Flow cytometric detection of phosphatidylserine expression on early apoptotic cells using fluorescein labelled Annexin V” (1995) J. Immunol. Methods 184:39-51) and cytotoxicity assays. Fluorometric microvolume assay technology can be used to identify the up or down regulation by a molecule that is targeted to the cell surface (Swartzman, “A homogeneous and multiplexed immunoassay for high-throughput screening using fluorometric microvolume assay technology”, (1999) Anal. Biochem. 271:143-51).

Labelled antibodies of the invention are useful as imaging biomarkers and probes by the various methods and techniques of biomedical and molecular imaging such as: (i) MRI (magnetic resonance imaging); (ii) MicroCT (computerized tomography); (iii) SPECT (single photon emission computed tomography); (iv) PET (positron emission tomography) Chen et al (2004) Bioconjugate Chem. 15:41-49; (v) bioluminescence; (vi) fluorescence; and (vii) ultrasound. Immunoscintigraphy is an imaging procedure in which antibodies labeled with radioactive substances are administered to an animal or human patient and a picture is taken of sites in the body where the antibody localizes (U.S. Pat. No. 6,528,624). Imaging biomarkers may be objectively measured and evaluated as an indicator of normal biological processes, pathogenic processes, or pharmacological responses to a therapeutic intervention.

Peptide labelling methods are well known. See Haugland, 2003, Molecular Probes Handbook of Fluorescent Probes and Research Chemicals, Molecular Probes, Inc.; Brinkley, 1992, Bioconjugate Chem. 3:2; Garman, (1997) Non-Radioactive Labelling: A Practical Approach, Academic Press, London; Means (1990) Bioconjugate Chem. 1:2; Glazer et al (1975) Chemical Modification of Proteins. Laboratory Techniques in Biochemistry and Molecular Biology (T. S. Work and E. Work, Eds.) American Elsevier Publishing Co., New York; Lundblad, R. L. and Noyes, C. M. (1984) Chemical Reagents for Protein Modification, Vols. I and II, CRC Press, New York; Pfleiderer, G. (1985) “Chemical Modification of Proteins”, Modern Methods in Protein Chemistry, H. Tschesche, Ed., Walter DeGryter, Berlin and New York; and Wong (1991) Chemistry of Protein Conjugation and Cross-linking, CRC Press, Boca Raton, Fla.); De Leon-Rodriguez et al (2004) Chem. Eur. J. 10:1149-1155; Lewis et al (2001) Bioconjugate Chem. 12:320-324; Li et al (2002) Bioconjugate Chem. 13:110-115; Mier et al (2005) Bioconjugate Chem. 16:240-237.

The labelled antibodies of the invention may also be used as an affinity purification agent. In this process, the labelled antibody is immobilized on a solid phase such a Sephadex resin or filter paper, using methods well known in the art. The immobilized antibody is contacted with a sample containing the antigen to be purified, and thereafter the support is washed with a suitable solvent that will remove substantially all the material in the sample except the antigen to be purified, which is bound to the immobilized polypeptide variant. Finally, the support is washed with another suitable solvent, such as glycine buffer, pH 5.0, that will release the antigen from the polypeptide variant.

In one aspect, an anti-GPC3 antibody of the invention binds to the same epitope on GPC3 bound by another GPC3 antibody. In another embodiment, a GPC3 antibody of the invention binds to the same epitope on GPC3 bound by a fragment (e.g., a Fab fragment) of a monoclonal antibody comprising the variable domains of SEQ ID NO: 2 and SEQ ID NO: 4) or a chimeric antibody comprising the variable domain of the monoclonal antibody comprising the sequences of SEQ ID NO: 2 and SEQ ID NO: 4 and constant domains from IgGI.

### C. Comprehensive Description of Anti-GPC3 Antibodies Encompassed by the Disclosure

In one embodiment, the present invention provides anti-GPC3 antibodies which may find use herein as diagnostic and/or therapeutic agents. Exemplary antibodies include polyclonal, monoclonal, chimeric, humanized, and human antibodies.

### 1. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOC12, or RTST=C=NR, where R and RI are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ½ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

### 2. Monoclonal Antibodies

A monoclonal antibody (mAb) to an antigen-of-interest can be prepared by using any technique known in the art. These include, but are not limited to, the hybridoma technique originally described by Kohler and Milstein (1975, Nature 256, 495-497), the human B cell hybridoma technique (Kozbor et al., 1983, Immunology Today 4:72), and the EBV-hybridoma technique (Cole et al., 1985, Monoclonal Antibodies and Cancer Therapy, Alan R. Liss, Inc., pp. 77-96). The Selected Lymphocyte Antibody Method (SLAM) (Babcook, J. S., et al., A novel strategy for generating monoclonal antibodies from single, isolated lymphocytes producing antibodies of defined specificities. Proc Natl Acad Sci USA, 1996. 93 (15): p. 7843-8.) and (McLean G R, Olsen O A, Watt I N, Rathanaswami P, Leslie K B, Babcook J S, Schrader J W. Recognition of human cytomegalovirus by human primary immunoglobulins identifies an innate foundation to an adaptive immune response. J Immunol. 2005 Apr. 15; 174 (8): 4768-78. Such antibodies may be of any immunoglobulin class including IgG, IgM, IgE, IgA, and IgD and any subclass thereof. The hybridoma producing the mAbs of use in this invention may be cultivated in vitro or in vivo.

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Coding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Coding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal, e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (CH and CO sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

### 3. Chimeric, Humanized, and Human Antibodies

In some embodiments, the anti-GPC3 antibody is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

The anti-GPC3 antibodies of the invention may further comprise humanized antibodies or human antibodies. Humanized forms of non-human (e.g., murine or rabbit) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. Reduction or elimination of a HAMA response is a significant aspect of clinical development of suitable therapeutic agents. See, e.g., Khaxzaeli et al., J. Natl. Cancer Inst. (1988), 80:937; Jaffers et al., Transplantation (1986), 41:572; Shawler et al., J. Immunol. (1985), 135:1530; Sears et al., J. Biol. Response Mod. (1984), 3:138; Miller et al., Blood (1983), 62:988; Hakimi et al., J. Immunol. (1991), 147:1352; Reichmann et al., Nature (1988), 332:323; Junghans et al., Cancer Res. (1990), 50:1495. As described herein, the invention provides antibodies that are humanized such that HAMA response is reduced or eliminated. Variants of these antibodies can further be obtained using routine methods known in the art, some of which are further described below. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

For example, an amino acid sequence from an antibody as described herein can serve as a starting (parent) sequence for diversification of the framework and/or hypervariable sequence(s). A selected framework sequence to which a starting hypervariable sequence is linked is referred to herein as an acceptor human framework. While the acceptor human frameworks may be from, or derived from, a human immunoglobulin (the VL and/or VH regions thereof), preferably the acceptor human frameworks are from, or derived from, a human consensus framework sequence as such frameworks have been demonstrated to have minimal, or no, immunogenicity in human patients.

Where the acceptor is derived from a human immunoglobulin, one may optionally select a human framework sequence that is selected based on its homology to the donor framework sequence by aligning the donor framework sequence with various human framework sequences in a collection of human framework sequences, and select the most homologous framework sequence as the acceptor.

In one embodiment, human consensus frameworks herein are from, or derived from, VH subgroup III and/or VL kappa subgroup I consensus framework sequences.

While the acceptor may be identical in sequence to the human framework sequence selected, whether that be from a human immunoglobulin or a human consensus framework, the present invention contemplates that the acceptor sequence may comprise pre-existing amino acid substitutions relative to the human immunoglobulin sequence or human consensus framework sequence. These pre-existing substitutions are preferably minimal; usually four, three, two or one amino acid differences only relative to the human immunoglobulin sequence or consensus framework sequence.

Hypervariable region residues of the non-human antibody are incorporated into the VL and/or VH acceptor human frameworks. For example, one may incorporate residues corresponding to the Kabat CDR residues, the Chothia hypervariable loop residues, the Abm residues, and/or contact residues. Optionally, the extended hypervariable region residues as follows are incorporated: 24-34 (LI), 50-56 (L2) and 89-97 (L3), 26-35B (HI), 50-65, 47-65 or 49-65 (H2) and 93-102, 94-102, or 95-102 (H3).

While “incorporation” of hypervariable region residues is discussed herein, it will be appreciated that this can be achieved in various ways, for example, nucleic acid encoding the desired amino acid sequence can be generated by mutating nucleic acid encoding the mouse variable domain sequence so that the framework residues thereof are changed to acceptor human framework residues, or by mutating nucleic acid encoding the human variable domain sequence so that the hypervariable domain residues are changed to non-human residues, or by synthesizing nucleic acid encoding the desired sequence, etc.

As described herein, hypervariable region-grafted variants may be generated by Kunkel mutagenesis of nucleic acid encoding the human acceptor sequences, using a separate oligonucleotide for each hypervariable region. Kunkel et al., Methods Enzymol. 154:367-382 (1987). Appropriate changes can be introduced within the framework and/or hypervariable region, using routine techniques, to correct and re-establish proper hypervariable region-antigen interactions.

Thus, in one embodiment, the invention provides a humanized antibody that elicits and/or is expected to elicit a human anti-mouse antibody response (HAMA) at a substantially reduced level compared to an antibody comprising the sequence of SEQ ID NO: 2 and 4 in a host subject. In another example, the invention provides a humanized antibody that elicits and/or is expected to elicit minimal or no human anti-mouse antibody response (HAMA). In one example, an antibody of the invention elicits anti-mouse antibody response that is at or less than a clinically-acceptable level.

A humanized antibody of the invention may comprise one or more human and/or human consensus non-hypervariable region (e.g., framework) sequences in its heavy and/or light chain variable domain. In some embodiments, one or more additional modifications are present within the human and/or human consensus non-hypervariable region sequences. In one embodiment, the heavy chain variable domain of an antibody of the invention comprises a human consensus framework sequence, which in one embodiment is the subgroup III consensus framework sequence. In one embodiment, an antibody of the invention comprises a variant subgroup III consensus framework sequence modified at least one amino acid position.

As is known in the art, and as described in greater detail herein, the amino acid position/boundary delineating a hypervariable region of an antibody can vary, depending on the context and the various definitions known in the art (as described below). Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions (as further defined below). The invention provides antibodies comprising modifications in these hybrid hypervariable positions. In one embodiment, these hypervariable positions include one or more positions 26-30, 33-35B, 47-49, 57-65, 93, 94 and 101-102 in a heavy chain variable domain. In one embodiment, these hybrid hypervariable positions include one or more of positions 24-29, 35-36, 46-49, 56 and 97 in a light chain variable domain. In one embodiment, an antibody of the invention comprises a human variant human subgroup consensus framework sequence modified at one or more hybrid hypervariable positions.

An antibody of the invention can comprise any suitable human or human consensus light chain framework sequences, provided the antibody exhibits the desired biological characteristics (e.g., a desired binding affinity). In one embodiment, an antibody of the invention comprises at least a portion (or all) of the framework sequence of human K light chain. In one embodiment, an antibody of the invention comprises at least a portion (or all) of human K subgroup I framework consensus sequence.

Phage(mid) display (also referred to herein as phage display in some contexts) can be used as a convenient and fast method for generating and screening many different potential variant antibodies in a library generated by sequence randomization. However, other methods for making and screening altered antibodies are available to the skilled person.

Phage(mid) display technology has provided a powerful tool for generating and selecting novel proteins which bind to a ligand, such as an antigen. Using the techniques of phage(mid) display allows the generation of large libraries of protein variants which can be rapidly sorted for those sequences that bind to a target molecule with high affinity. Nucleic acids encoding variant polypeptides are generally fused to a nucleic acid sequence encoding a viral coat protein, such as the gene III protein or the gene VIII protein. Monovalent phagemid display systems where the nucleic acid sequence encoding the protein or polypeptide is fused to a nucleic acid sequence encoding a portion of the gene III protein have been developed. (Bass, S., Proteins, 8:309 (1990); Lowman and Wells, Methods: A Companion to Methods in Enzymology, 3:205 (1991)). In a monovalent phagemid display system, the gene fusion is expressed at low levels and wild type gene III proteins are also expressed so that infectivity of the particles is retained. Methods of generating peptide libraries and screening those libraries have been disclosed in many patents (e.g. U.S. Pat. Nos. 5,723,286, 5,432,018, 5,580,717, 5,427,908 and 5,498,530).

Libraries of antibodies or antigen binding polypeptides have been prepared in a number of ways including by altering a single gene by inserting random DNA sequences or by cloning a family of related genes. Methods for displaying antibodies or antigen binding fragments using phage(mid) display have been described in U.S. Pat. Nos. 5,750,373, 5,733,743, 5,837,242, 5,969,108, 6,172,197, 5,580,717, and 5,658,727. The library is then screened for expression of antibodies or antigen binding proteins with the desired characteristics.

Methods of substituting an amino acid of choice into a template nucleic acid are well established in the art, some of which are described herein. For example, hypervariable region residues can be substituted using the Kunkel method. See, e.g., Kunkel et al., Methods Enzymol. 154:367-382 (1987).

The sequence of oligonucleotides includes one or more of the designed codon sets for the hypervariable region residues to be altered. A codon set is a set of different nucleotide triplet sequences used to encode desired variant amino acids. Codon sets can be represented using symbols to designate particular nucleotides or equimolar mixtures of nucleotides as shown in below according to the IUB code.

### IUB Codes

### G Guanine

### A Adenine

### T Thymine

### C Cytosine

### R (A or G)

### Y (C or T)

### M (A or C)

### K (G or T)

### S (C or G)

### W (A or T)

### H (A or C or T)

### B (C or G or T)

### V (A or C or G)

### D (A or G or T) H

### N (A or C or G or T)

For example, in the codon set DVK, D can be nucleotides A or G or T; V can be A or G or C; and K can be G or T. This codon set can present 18 different codons and can encode amino acids Ala, Trp, Tyr, Lys, Thr, Asn, Lys, Ser, Arg, Asp, Glu, Gly, and Cys.

Oligonucleotide or primer sets can be synthesized using standard methods. A set of oligonucleotides can be synthesized, for example, by solid phase synthesis, containing sequences that represent all possible combinations of nucleotide triplets provided by the codon set and that will encode the desired group of amino acids. Synthesis of oligonucleotides with selected nucleotide “degeneracy” at certain positions is well known in that art. Such sets of nucleotides having certain codon sets can be synthesized using commercial nucleic acid synthesizers (available from, for example, Applied Biosystems, Foster City, Calif), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can include restriction enzyme sites for cloning purposes.

In one method, nucleic acid sequences encoding variant amino acids can be created by oligonucleotide-mediated mutagenesis. This technique is well known in the art as described by Zoller et al. Nucleic Acids Res. 10:6487-6504 (1987). Briefly, nucleic acid sequences encoding variant amino acids are created by hybridizing an oligonucleotide set encoding the desired codon sets to a DNA template, where the template is the single-stranded form of the plasmid containing a variable region nucleic acid template sequence. After hybridization, DNA polymerase is used to synthesize an entire second complementary strand of the template that will thus incorporate the oligonucleotide primer, and will contain the codon sets as provided by the oligonucleotide set.

Generally, oligonucleotides of at least 25 nucleotides in length are used. An optimal oligonucleotide will have 12 to 15 nucleotides that are completely complementary to the template on either side of the nucleotide(s) coding for the mutation(s). This ensures that the oligonucleotide will hybridize properly to the single-stranded DNA template molecule. The oligonucleotides are readily synthesized using techniques known in the art such as that described by Crea et al., Proc. Nat'l. Acad. Sci. USA, 75:5765 (1978).

The DNA template is generated by those vectors that are either derived from bacteriophage MI 3 vectors (the commercially available MI 3 mp 18 and MI 3 mp 19 vectors are suitable), or those vectors that contain a single-stranded phage origin of replication as described by Viera et al., Meth. Enzymol., 153:3 (1987). Thus, the DNA that is to be mutated can be inserted into one of these vectors in order to generate single-stranded template.

Production of the single-stranded template is described in sections 4.21-4.41 of Sambrook et al., above. To alter the native DNA sequence, the oligonucleotide is hybridized to the single stranded template under suitable hybridization conditions. A DNA polymerizing enzyme, usually T7 DNA polymerase or the Klenow fragment of DNA polymerase I, is then added to synthesize the complementary strand of the template using the oligonucleotide as a primer for synthesis. A heteroduplex molecule is thus formed such that one strand of DNA encodes the mutated form of gene 1, and the other strand (the original template) encodes the native, unaltered sequence of gene 1. This heteroduplex molecule is then transformed into a suitable host cell, usually a prokaryote such as E. coli JM101. After growing the cells, they are plated onto agarose plates and screened using the oligonucleotide primer radiolabelled with a 32-Phosphate to identify the bacterial colonies that contain the mutated DNA.

The method described immediately above may be modified such that a homoduplex molecule is created wherein both strands of the plasmid contain the mutation(s). The modifications are as follows: The single stranded oligonucleotide is annealed to the single-stranded template as described above. A mixture of three deoxyribonucleotides, deoxyriboadenosine (dATP), deoxyriboguanosine (dGTP), and deoxyribothymidine (dTT), is combined with a modified thiodeoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion.

After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell.

As indicated previously the sequence of the oligonucleotide set is of sufficient length to hybridize to the template nucleic acid and may also, but does not necessarily, contain restriction sites. The DNA template can be generated by those vectors that are either derived from bacteriophage MI 3 vectors or vectors that contain a single-stranded phage origin of replication as described by Viera et al. Meth. Enzymol., 153:3 (1987). Thus, the DNA that is to be mutated must be inserted into one of these vectors in order to generate single-stranded template. Production of the single-stranded template is described in sections 4.21-4.41 of Sambrook et al., supra.

According to another method, antigen binding may be restored during humanization of antibodies through the selection of repaired hypervariable regions (See application Ser. No. 11/061,841, filed Feb. 18, 2005). The method includes incorporating non-human hypervariable regions onto an acceptor framework and further introducing one or more amino acid substitutions in one or more hypervariable regions without modifying the acceptor framework sequence. Alternatively, the introduction of one or more amino acid substitutions may be accompanied by modifications in the acceptor framework sequence.

According to another method, a library can be generated by providing upstream and downstream oligonucleotide sets, each set having a plurality of oligonucleotides with different sequences, the different sequences established by the codon sets provided within the sequence of the oligonucleotides. The upstream and downstream oligonucleotide sets, along with a variable domain template nucleic acid sequence, can be used in a polymerase chain reaction to generate a “library” of PCR products. The PCR products can be referred to as “nucleic acid cassettes”, as they can be fused with other related or unrelated nucleic acid sequences, for example, viral coat proteins and dimerization domains, using established molecular biology techniques.

The sequence of the PCR primers includes one or more of the designed codon sets for the solvent accessible and highly diverse positions in a hypervariable region. As described above, a codon set is a set of different nucleotide triplet sequences used to encode desired variant amino acids.

Antibody selectants that meet the desired criteria, as selected through appropriate screening/selection steps can be isolated and cloned using standard recombinant techniques.

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-GPC3 antibody are contemplated. For example, the humanized antibody may be an antibody fragment, such as a Fab. Alternatively, the humanized antibody may be an intact antibody, such as an intact IgGI antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as MI 3 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

In another embodiment, the antibodies of this disclosure are human monoclonal antibodies. Such human monoclonal antibodies directed against GPC3 can be generated using transgenic or transchromosomic mice carrying parts of the human immune system rather than the mouse system. These transgenic and transchromosomic mice include mice referred to herein as the HuMAb Mouse™ and KM Mouse™, respectively, and are collectively referred to herein as “human Ig mice.” The HuMAb Mouse™ (Medarex, Inc.) contains human immunoglobulin gene miniloci that encode unrearranged human heavy (μ and γ) and K light chain immunoglobulin sequences, together with targeted mutations that inactivate the endogenous μ and κ chain loci (see e.g., Lonberg, et al. (1994) Nature 368 (6474): 856-859). Accordingly, the mice exhibit reduced expression of mouse IgM or κ, and in response to immunization, the introduced human heavy and light chain transgenes undergo class switching and somatic mutation to generate high affinity human IgGK monoclonal antibodies (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13:65-93, and Harding, F. and Lonberg, N. (1995) Ann. N.Y. Acad. Sci. 764:536-546). Preparation and use of the HuMAb Mouse™, and the genomic modifications carried by such mice, is further described in Taylor, L. et al. (1992) Nucleic Acids Research 20:6287-6295; Chen, J. et al. (1993) International Immunology 5:647-656; Tuaillon et al. (1993) Proc. Natl. Acad. Sci. USA 90:3720-3724; Choi et al. (1993) Nature Genetics 4:117-123; Chen, L et al. (1993) EMBO J. 12:821-830; Tuaillon et al., (1994) J. Immunol. 152:2912-2920; Taylor, L. et al. (1994) International Immunology 6:579-591; and Fishwild, D. et al. (1996) Nature Biotechnology 14:845-851, the contents of all of which are hereby specifically incorporated by reference in their entirety. See further, U.S. Pat. Nos. 5,545,806; 5,569,825; 5,625, 126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al. In another embodiment, human antibodies of this disclosure can be raised using a mouse that carries human immunoglobulin sequences on transgenes and transchomosomes, such as a mouse that carries a human heavy chain transgene and a human light chain transchromosome. This mouse is referred to herein as a “KM Mouse™” and is described in detail in PCT Publication WO 02/43478 to Ishida et al.

Still further, alternative transgenic animal systems expressing human immunoglobulin genes are available in the art and can be used to raise anti-GPC3 antibodies of this disclosure. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-GPC3 antibodies of this disclosure. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise anti-GPC3 antibodies of this disclosure.

### 4. Antibody Fragments

In certain circumstances there are advantages of using antibody fragments, rather than whole antibodies. The smaller size of the fragments allows for rapid clearance, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)). According to another approach, F(ab′)2 fragments can be isolated directly from recombinant host cell culture. Fab and F(ab′)2 fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. Nos. 5,571,894; and 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example.

In one embodiment, an anti-GPC3 antibody derived scFv is used in a CAR modified immune cell, preferably a CAR-T or CAR-NK cell disclosed herein. Included among anti-GPC3 antibody fragments are portions of anti-GPC3 antibodies (and combinations of portions of anti-GPC3 antibodies, for example, scFv) that may be used as targeting arms, directed to GPC3 tumor epitope, in chimeric antigenic receptors of CAR-T or CAR-NK cells. Such fragments are not necessarily proeteolytic fragments but rather portions of polypeptide sequences that can confer affinity for target.

### 5. Bispecific Antibodies

Bispecific antibodies are antibodies that have binding specificities for at least two different epitopes. Exemplary bispecific antibodies may bind to two different epitopes of a GPC3 protein as described herein. Other such antibodies may combine a GPC3 binding site with a binding site for another protein. Alternatively, an anti-GPC3 arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the GPC3-expressing cell. Bispecific antibodies may also be used to localize cytotoxic agents to cells which express GPC3. These antibodies possess a GPC3-binding arm and an arm which binds the cytotoxic agent (e.g., saporin, anti-interferon-a, vinca alkaloid, ricin A chain, methotrexate or radioactive isotope hapten). Bispecific antibodies can be prepared as full length antibodies or antibody fragments (e.g., F(ab′)2 bispecific antibodies).

WO 96/16673 describes a bispecific anti-ErbB2/anti-FcYRIII antibody and U.S. Pat. No. 5,837,234 discloses a bispecific anti-ErbB2/anti-FcγRI antibody. A bispecific anti-ErbB2/Fca antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure.

Purification of the correct molecule, which is usually done by affinity chromatography steps, is rather cumbersome, and the product yields are low. Similar procedures are disclosed in WO 93/08829, and in Traunecker et al., EMBO J. 10:3655-3659 (1991).

### 6. Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody.

Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgGI, IgG2, IgG3, or IgG4) that is responsible for increasing the in vivo serum half-life of the IgG molecule.

### D. Certain Methods of Making Antibodies

1. Screening for Anti-GPC3 Antibodies with the Desired Properties

Techniques for generating antibodies that bind to GPC3 polypeptides have been described above. For example, the invention provides a method of making a GPC3 antibody (which, as defined herein includes full length and fragments thereof), said method comprising expressing in a suitable host cell a recombinant vector of the invention encoding said antibody (or fragment thereof), and recovering said antibody. One may further select antibodies with certain biological characteristics, as desired.

The growth inhibitory effects of an anti-GPC3 antibody of the invention may be assessed by methods known in the art, e.g., using cells which express a GPC3 polypeptide either endogenously or following transfection with the GPC3 gene. For example, appropriate tumor cell lines and GPC3-transfected cells may be treated with an anti-GPC3 monoclonal antibody of the invention at various concentrations for a few days (e.g., 2-7) days and stained with crystal violet or MTT or analyzed by some other colorimetric assay. Another method of measuring proliferation would be by comparing 3H-thymidine uptake by the cells treated in the presence or absence an anti-GPC3 antibody of the invention. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of tumor cells in vivo can be determined in various ways known in the art. The tumor cell may be one that overexpresses a GPC3 polypeptide. The anti-GPC3 antibody will inhibit cell proliferation of a GPC3-expressing tumor cell in vitro or in vivo by about 25-100% compared to the untreated tumor cell, more preferably, by about 30-100%, and even more preferably by about 50-100% or 70-100%, in one embodiment, at an antibody concentration of about 0.5 to 30 μg ml. Growth inhibition can be measured at an antibody concentration of about 0.5 to 30 μg ml or about 0.5 nM to 200 nM in cell culture, where the growth inhibition is determined 1-10 days after exposure of the tumor cells to the antibody. The antibody is growth inhibitory in vivo if administration of the anti-GPC3 antibody at about 1 μg/kg to about 100 mg/kg body weight results in reduction in tumor size or reduction of tumor cell proliferation within about 5 days to 3 months from the first administration of the antibody, preferably within about 5 to 30 days.

To select for an anti-GPC3 antibody which induces cell death, loss of membrane integrity as indicated by, e.g., propidium iodide (PI), trypan blue or 7AAD uptake may be assessed relative to control. A PI uptake assay can be performed in the absence of complement and immune effector cells. GPC3 polypeptide-expressing tumor cells are incubated with medium alone or medium containing the appropriate anti-GPC3 antibody (e.g, at about 10 μg/ml). The cells are incubated for a 3 day time period. Following each treatment, cells are washed and aliquoted into 35 mm strainer-capped 12×75 tubes (1 ml per tube, 3 tubes per treatment group) for removal of cell clumps. Tubes then receive PI (IC\hat{ }g/ml). Samples may be analyzed using a FACSCAN® flow cytometer and FACSCONVERT® CellQuest software (Becton Dickinson). Those anti-GPC3 antibodies that induce statistically significant levels of cell death as determined by PI uptake may be selected as cell death-inducing anti-GPC3 antibodies.

To screen for antibodies which bind to an epitope on a GPC3 polypeptide bound by an antibody of interest, a routine cross-blocking assay such as that described in Antibodies, A Laboratory Manual, Cold Spring Harbor Laboratory, Ed Harlow and David Lane (1988), can be performed. This assay can be used to determine if a test antibody binds the same site or epitope as a known anti-GPC3 antibody. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of a GPC3 polypeptide can be used in competition assays with the test antibodies or with a test antibody and an antibody with a characterized or known epitope.

Briefly, in one aspect, the disclosure provides a method for identifying the epitope of an agent that can be used in the disclosed IHC IVD assay and/or any other methods as herein disclosed. In some embodiments, the agent is 204. An epitope can include at least 3, 4, 5, 6, 7, 8, 9, 10, 11, 12, 13, 14, 15, 16, 17, 18, 19, or 20 amino acids in a unique spatial conformation. Epitopes can be formed both from contiguous amino acids or noncontiguous amino acids juxtaposed by tertiary folding of a protein. Epitopes formed from contiguous amino acids can be typically retained on exposure to denaturing solvents whereas epitopes formed by tertiary folding can be typically lost on treatment with denaturing solvents.

Epitope mapping can be performed to identify the linear or non-linear, discontinuous amino acid sequence(s), i.e. the epitope, that is recognized by an activating agent of interest, such as the 204 antibody. A general approach for epitope mapping can require the expression of the full-length polypeptide sequence that is recognized by an antibody or ligand of interest, as well as various fragments, i.e., truncated forms of the polypeptide sequence, generally in a heterologous expression system. These various recombinant polypeptide sequences or fragments thereof (e.g., fused with an N-terminal protein (e.g., GFP)) can then be used to determine if the antibody or ligand of interest is capable of binding to one or more of the truncated forms of the polypeptide sequence. Through the use of reiterative truncation and the generation of recombinant polypeptide sequences with overlapping amino acid regions, it is possible to identify the region of the polypeptide sequence that is recognized by the antibody of interest (see, e.g., Epitope Mapping Protocols in Methods in Molecular Biology, Vol. 66, Glenn E. Morris, Ed (1996)). The methods rely on the ability of an agent such as an antibody of interest to bind to sequences that have been recreated from epitope libraries, such as epitope libraries derived from, synthetic peptide arrays on membrane supports, combinatorial phage display peptide libraries. The epitope libraries then provide a range of possibilities that are screened against an antibody. Additionally, site specific mutagenesis, or random Ala scan, targeting one or more residues of an epitope can be pursued to confirm the identity of an epitope.

A library of epitopes can be created by synthetically designing various possible recombinations of GPC3 as cDNA constructs and expressing them in a suitable system. For instance, a plurality of GPC3 gene segments (e.g., various sequences corresponding to the N-terminal alpha chain, various sequences corresponding to the C-terminal beta chain, and the like) can be synthetically designed. Alternatively, the selected sequences can also ordered as synthetic genes and cloned into suitable vectors. In other cases, various GPC3 sequences can be amplified out of Total RNA extracted from human normal and malignant tissue, preferably malignant tissue where GPC3 is expressed at a higher level than normal tissues.

The host system can be any suitable expression system such as 293 cells, insect cells, or a suitable in-vitro translation system. The plurality of various possible recombinations of synthetically designed GPC3 gene segments transfected into a host system can provide, for instance, more than 25, 30, 35, 40, 45, 50, 55, 60, 65, 70, 75, 80, 85, or 90 possible pairing combinations of GPC3. The binding of an agent to one of the epitopes in the previously described library can be detected by contacting a labeled antibody, such as 204, with an epitope of the library and detecting a signal from the label.

For epitope mapping, computational algorithms have also been developed which have been shown to map conformational discontinuous epitopes. Conformational epitopes can be identified by determining spatial conformation of amino acids with methods that include, e.g., x-ray crystallography and 2-dimensional nuclear magnetic resonance. Some epitope mapping methods, such as, x-ray analyses of crystals of antigen: antibody complexes can provide atomic resolution of the epitope. In other cases, computational combinatorial methods for epitope mapping can be employed to model a potential epitope based on the sequence of the antibody, such as 204 antibody. In such cases, the antigen binding portion of the antibody is sequenced, and computation models are used to reconstruct and predict a potential binding site of the antibody.

In some cases the disclosure provides a method of determining an epitope of GPC3 that is specifically recognized by 204, or antigen-binding fragments thereof, comprising: (a) preparing a library of epitopes from GPC3; (b) contacting the library of epitopes with the 204 antibody, or antigen-binding fragments thereof; and (b) identifying the amino acid sequence of at least one epitope in the library of epitopes that is bound by the antibody. In one instance, the antibody is attached to a solid support. The library of epitopes can comprise sequences that correspond to continuous and discontinuous epitopes of GPC3. In some cases, the library of epitopes comprises fragments from GPC3 ranging from about 10 amino acids to about 30 amino acids in length, from about 10 amino acids to about 20 amino acids in length, or from about 5 amino acids to about 12 amino acids in length. In some cases, the 204 antibody, or antigen-binding fragment thereof, is labeled and the label is a radioactive molecule, a luminescent molecule, a fluorescent molecule, an enzyme, or biotin.

A high level epitope mapping study relying on western blotting and protease cleavage of GPC3 is described infra at Example 2. Briefly, following protease-cleavage (e.g., A Disintegrin and Metalloprotease, or ADAM) of GPC3, samples are subjected to western blotting analysis using an antibody for which the corresponding GPC3 epitope is known (e.g., GC33), as well as an antibody (e.g., 204) for which the corresponding GPC3 epitope is unknown. Differential recognition of protease-cleaved GPC3 fragments by the different antibodies can provide information as to the potential binding site of the target antibody, and can be used as a starting point in the above-referenced epitope mapping methodologies.

In addition, candidate antibodies may also be screened for function using one or more of the following: in vivo screening for inhibition of metastasis, inhibition of chemotaxis by an in vitro method (e.g., Huntsman et al. U.S. 2010/0061978, incorporated herein by reference in its entirety), inhibition of vascularization, inhibition of tumour growth, and decrease in tumor size.

### 2. Certain Library Screening Methods

Anti-GPC3 antibodies of the invention can be made by using combinatorial libraries to screen for antibodies with the desired activity or activities. For example, a variety of methods are known in the art for generating phage display libraries and screening such libraries for antibodies possessing the desired binding characteristics. Such methods are described generally in Hoogenboom et al. (2001) in Methods in Molecular Biology 178:1-37 (O'Brien et al., ed., Human Press, Totowa, NJ), and in certain embodiments, in Lee et al. (2004) J. Mol. Biol. 340:1073-1093.

In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the anti-GPC3 antibodies of the invention can be obtained by designing a suitable antigen screening procedure to select for the phage clone of interest followed by construction of a full length anti-GPC3 antibody clone using the Fv sequences from the phage clone of interest and suitable constant region (Fc) sequences described in Kabat et al., Sequences of Proteins of Immunological Interest, Fifth Edition, NIH Publication 91-3242, Bethesda MD (1991), vols. 1-3.

In certain embodiments, the antigen-binding domain of an antibody is formed from two variable (V) regions of about 110 amino acids, one each from the light (VL) and heavy (VH) chains, that both present three hypervariable loops (HVRs) or complementarity-determining regions (CDRs). Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described in Winter et al., Ann. Rev. Immunol., 12:433-455 (1994). As used herein, scFv encoding phage clones and Fab encoding phage clones are collectively referred to as “Fv phage clones” or “Fv clones.”

Repertoires of VH and VL genes can be separately cloned by polymerase chain reaction (PCR) and recombined randomly in phage libraries, which can then be searched for antigen-binding clones as described in Winter et al., Ann. Rev. Immunol., 12:433-455 (1994). Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12:725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described by Hoogenboom and Winter, J. Mol. Biol., 227:381-388 (1992).

In certain embodiments, filamentous phage is used to display antibody fragments by fusion to the minor coat protein pill. The antibody fragments can be displayed as single chain Fv fragments, in which VH and VL domains are connected on the same polypeptide chain by a flexible polypeptide spacer, e.g. as described by Marks et al., J. Mol. Biol., 222:581-597 (1991), or as Fab fragments, in which one chain is fused to pill and the other is secreted into the bacterial host cell periplasm where assembly of a Fab-coat protein structure which becomes displayed on the phage surface by displacing some of the wild type coat proteins, e.g. as described in Hoogenboom et al., Nucl. Acids Res., 19:4133-4137 (1991).

In general, nucleic acids encoding antibody gene fragments are obtained from immune cells harvested from humans or animals. If a library biased in favor of anti-GPC3 clones is desired, the subject is immunized with GPC3 to generate an antibody response, and spleen cells and/or circulating B cells other peripheral blood lymphocytes (PBLs) are recovered for library construction. In a preferred embodiment, a human antibody gene fragment library biased in favor of anti-GPC3 clones is obtained by generating an anti-GPC3 antibody response in transgenic mice carrying a functional human immunoglobulin gene array (and lacking a functional endogenous antibody production system) such that GPC3 immunization gives rise to B cells producing human antibodies against GPC3. The generation of human antibody-producing transgenic mice is described below.

Additional enrichment for anti-GPC3 reactive cell populations can be obtained by using a suitable screening procedure to isolate B cells expressing GPC3-specific membrane bound antibody, e.g., by cell separation using GPC3 affinity chromatography or adsorption of cells to fluorochrome-labeled GPC3 followed by flow-activated cell sorting (FACS).

Alternatively, the use of spleen cells and/or B cells or other PBLs from an unimmunized donor provides a better representation of the possible antibody repertoire, and also permits the construction of an antibody library using any animal (human or non-human) species in which GPC3 is not antigenic. For libraries incorporating in vitro antibody gene construction, stem cells are harvested from the subject to provide nucleic acids encoding unrearranged antibody gene segments. The immune cells of interest can be obtained from a variety of animal species, such as human, mouse, rat, lagomorpha, luprine, canine, feline, porcine, bovine, equine, and avian species, etc.

Nucleic acid encoding antibody variable gene segments (including VH and VL segments) are recovered from the cells of interest and amplified. In the case of rearranged VH and VL gene libraries, the desired DNA can be obtained by isolating genomic DNA or mRNA from lymphocytes followed by polymerase chain reaction (PCR) with primers matching the 5′ and 3′ ends of rearranged VH and VL genes as described in Orlandi et al., Proc. Natl. Acad. Sci. (USA), 86:3833-3837 (1989), thereby making diverse V gene repertoires for expression. The V genes can be amplified from cDNA and genomic DNA, with back primers at the 5′ end of the exon encoding the mature V-domain and forward primers based within the J-segment as described in Orlandi et al. (1989) and in Ward et al., Nature, 341:544-546 (1989). However, for amplifying from cDNA, back primers can also be based in the leader exon as described in Jones et al., Biotechnol., 9:88-89 (1991), and forward primers within the constant region as described in Sastry et al., Proc. Natl. Acad. Sci. (USA), 86:5728-5732 (1989). To maximize complementarity, degeneracy can be incorporated in the primers as described in Orlandi et al. (1989) or Sastry et al. (1989). In certain embodiments, library diversity is maximized by using PCR primers targeted to each V-gene family in order to amplify all available VH and VL arrangements present in the immune cell nucleic acid sample, e.g. as described in the method of Marks et al., J. Mol. Biol., 222:581-597 (1991) or as described in the method of Orum et al., Nucleic Acids Res., 21:4491-4498 (1993). For cloning of the amplified DNA into expression vectors, rare restriction sites can be introduced within the PCR primer as a tag at one end as described in Orlandi et al. (1989), or by further PCR amplification with a tagged primer as described in Clackson et al., Nature, 352:624-628 (1991).

Repertoires of synthetically rearranged V genes can be derived in vitro from V gene segments. Most of the human VH-gene segments have been cloned and sequenced (reported in Tomlinson et al., J. Mol. Biol., 227:776-798 (1992)), and mapped (reported in Matsuda et al., Nature Genet, 3:88-94 (1993); these cloned segments (including all the major conformations of the HI and H2 loop) can be used to generate diverse VH gene repertoires with PCR primers encoding H3 loops of diverse sequence and length as described in Hoogenboom and Winter, J. Mol. Biol., 227:381-388 (1992). VH repertoires can also be made with all the sequence diversity focused in a long H3 loop of a single length as described in Barbas et al., Proc. Natl. Acad. Sci. USA, 89:4457-4461 (1992). Human VK and vλ segments have been cloned and sequenced (reported in Williams and Winter, Eur. J. Immunol., 23:1456-1461 (1993)) and can be used to make synthetic light chain repertoires. Synthetic V gene repertoires, based on a range of VH and VL folds, and L3 and H3 lengths, will encode antibodies of considerable structural diversity. Following amplification of V-gene encoding DNAs, germline V-gene segments can be rearranged in vitro according to the methods of Hoogenboom and Winter, J. Mol. Biol., 227:381-388 (1992).

Repertoires of antibody fragments can be constructed by combining VH and VL gene repertoires together in several ways. Each repertoire can be created in different vectors, and the vectors recombined in vitro, e.g., as described in Hogrefe et al., Gene, 128:119-126 (1993), or in vivo by combinatorial infection, e.g., the IoxP system described in Waterhouse et al., Nucl. Acids Res., 21:2265-2266 (1993). The in vivo recombination approach exploits the two-chain nature of Fab fragments to overcome the limit on library size imposed by E. coli transformation efficiency. Naive VH and VL repertoires are cloned separately, one into a phagemid and the other into a phage vector. The two libraries are then combined by phage infection of phagemid-containing bacteria so that each cell contains a different combination and the library size is limited only by the number of cells present (about 1012 clones). Both vectors contain in vivo recombination signals so that the VH and VL genes are recombined onto a single replicon and are co-packaged into phage virions. These huge libraries provide large numbers of diverse antibodies of good affinity (Kd⁻¹ of about 10⁻⁸ M).

Alternatively, the repertoires may be cloned sequentially into the same vector, e.g. as described in Barbas et al., Proc. Natl. Acad. Sci. USA, 88:7978-7982 (1991), or assembled together by PCR and then cloned, e.g. as described in Clackson et al., Nature, 352:624-628 (1991). PCR assembly can also be used to join VH and VL DNAs with DNA encoding a flexible peptide spacer to form single chain Fv (scFv) repertoires. In yet another technique, “in cell PCR assembly” is used to combine VH and VL genes within lymphocytes by PCR and then clone repertoires of linked genes as described in Embleton et al., Nucl. Acids Res., 20:3831-3837 (1992).

The antibodies produced by naive libraries (either natural or synthetic) can be of moderate affinity (Kd⁻¹ of about 106 to 107 M-I), but affinity maturation can also be mimicked in vitro by constructing and reselecting from secondary libraries as described in Winter et al. (1994), supra. For example, mutation can be introduced at random in vitro by using error-prone polymerase (reported in Leung et al., Technique, 1:11-15 (1989)) in the method of Hawkins et al., J. Mol. Biol., 226:889-896 (1992) or in the method of Gram et al., Proc. Natl. Acad. Sci USA, 89:3576-3580 (1992). Additionally, affinity maturation can be performed by randomly mutating one or more CDRs, e.g. using PCR with primers carrying random sequence spanning the CDR of interest, in selected individual Fv clones and screening for higher affinity clones. WO 9607754 (published 14 Mar. 1996) described a method for inducing mutagenesis in a complementarity determining region of an immunoglobulin light chain to create a library of light chain genes. Another effective approach is to recombine the VH or VL domains selected by phage display with repertoires of naturally occurring V domain variants obtained from unimmunized donors and screen for higher affinity in several rounds of chain reshuffling as described in Marks et al., Biotechnol., 10:779-783 (1992). This technique allows the production of antibodies and antibody fragments with affinities of about 10⁻⁹ M or less.

Screening of the libraries can be accomplished by various techniques known in the art. For example, GPC3 can be used to coat the wells of adsorption plates, expressed on host cells affixed to adsorption plates or used in cell sorting, or conjugated to biotin for capture with streptavidin-coated beads, or used in any other method for panning phage display libraries.

The phage library samples are contacted with immobilized GPC3 under conditions suitable for binding at least a portion of the phage particles with the adsorbent. Normally, the conditions, including pH, ionic strength, temperature and the like are selected to mimic physiological conditions. The phages bound to the solid phase are washed and then eluted by acid, e.g. as described in Barbas et al., Proc. Natl. Acad. Sci USA, 88:7978-7982 (1991), or by alkali, e.g. as described in Marks et al., J. Mol. Biol., 222:581-597 (1991), or by GPC3 antigen competition, e.g. in a procedure similar to the antigen competition method of Clackson et al., Nature, 352:624-628 (1991). Phages can be enriched 20-1,000-fold in a single round of selection. Moreover, the enriched phages can be grown in bacterial culture and subjected to further rounds of selection.

The efficiency of selection depends on many factors, including the kinetics of dissociation during washing, and whether multiple antibody fragments on a single phage can simultaneously engage with antigen. Antibodies with fast dissociation kinetics (and weak binding affinities) can be retained by use of short washes, multivalent phage display and high coating density of antigen in solid phase. The high density not only stabilizes the phage through multivalent interactions, but favors rebinding of phage that has dissociated. The selection of antibodies with slow dissociation kinetics (and good binding affinities) can be promoted by use of long washes and monovalent phage display as described in Bass et al., Proteins, 8:309-314 (1990) and in WO 92/09690, and a low coating density of antigen as described in Marks et al., Biotechnol., 10:779-783 (1992).

It is possible to select between phage antibodies of different affinities, even with affinities that differ slightly, for GPC3. However, random mutation of a selected antibody (e.g. as performed in some affinity maturation techniques) is likely to give rise to many mutants, most binding to antigen, and a few with higher affinity. With limiting GPC3, rare high affinity phage could be competed out. To retain all higher affinity mutants, phages can be incubated with excess biotinylated GPC3, but with the biotinylated GPC3 at a concentration of lower molarity than the target molar affinity constant for GPC3. The high affinity-binding phages can then be captured by streptavidin-coated paramagnetic beads. Such “equilibrium capture” allows the antibodies to be selected according to their affinities of binding, with sensitivity that permits isolation of mutant clones with as little as two-fold higher affinity from a great excess of phages with lower affinity. Conditions used in washing phages bound to a solid phase can also be manipulated to discriminate on the basis of dissociation kinetics.

Anti-GPC3 clones may be selected based on activity. In certain embodiments, the invention provides anti-GPC3 antibodies that bind to living cells that naturally express GPC3. In one embodiment, the invention provides anti-GPC3 antibodies that block the binding between a GPC3 ligand and GPC3, but do not block the binding between a GPC3 ligand and a second protein. Fv clones corresponding to such anti-GPC3 antibodies can be selected by (1) isolating anti-GPC3 clones from a phage library as described above, and optionally amplifying the isolated population of phage clones by growing up the population in a suitable bacterial host; (2) selecting GPC3 and a second protein against which blocking and non-blocking activity, respectively, is desired; (3) adsorbing the anti-GPC3 phage clones to immobilized GPC3; (4) using an excess of the second protein to elute any undesired clones that recognize GPC3-binding determinants which overlap or are shared with the binding determinants of the second protein; and (5) eluting the clones which remain adsorbed following step (4). Optionally, clones with the desired blocking/non-blocking properties can be further enriched by repeating the selection procedures described herein one or more times.

DNA encoding hybridoma-derived monoclonal antibodies or phage display Fv clones of the invention is readily isolated and sequenced using conventional procedures (e.g. by using oligonucleotide primers designed to specifically amplify the heavy and light chain coding regions of interest from hybridoma or phage DNA template). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of the desired monoclonal antibodies in the recombinant host cells. Review articles on recombinant expression in bacteria of antibody-encoding DNA include Skerra et al., Curr. Opinion in Immunol., 5:256 (1993) and Pluckthun, Immunol. Revs, 130:151 (1992).

DNA encoding the Fv clones of the invention can be combined with known DNA sequences encoding heavy chain and/or light chain constant regions (e.g. the appropriate DNA sequences can be obtained from Kabat et al., supra) to form clones encoding full or partial length heavy and/or light chains. It will be appreciated that constant regions of any isotype can be used for this purpose, including IgG, IgM, IgA, IgD, and IgE constant regions, and that such constant regions can be obtained from any human or animal species. An Fv clone derived from the variable domain DNA of one animal (such as human) species and then fused to constant region DNA of another animal species to form coding sequence(s) for “hybrid,” full length heavy chain and/or light chain is included in the definition of “chimeric” and “hybrid” antibody as used herein. In certain embodiments, an Fv clone derived from human variable DNA is fused to human constant region DNA to form coding sequence(s) for full- or partial-length human heavy and/or light chains.

DNA encoding anti-GPC3 antibody derived from a hybridoma can also be modified, for example, by substituting the coding sequence for human heavy- and light-chain constant domains in place of homologous murine sequences derived from the hybridoma clone (e.g. as in the method of Morrison et al., Proc. Natl. Acad. Sci. USA, 81:6851-6855 (1984)). DNA encoding a hybridoma- or Fv clone-derived antibody or fragment can be further modified by covalently joining to the immunoglobulin coding sequence all or part of the coding sequence for a non-immunoglobulin polypeptide. In this manner, “chimeric” or “hybrid” antibodies are prepared that have the binding specificity of the Fv clone or hybridoma clone-derived antibodies of the invention.

### 3. Generation of Antibodies Using CAR T-Cells

Anti-GPC3 antibodies of the invention can be made by using CAR T-cell platforms to screen for antibodies with the desired activity or activities. Chimeric antigen receptors (CARs) are composed of an extracellular antigen recognition domain (usually a single-chain variable fragment (scFv) antibody) attached to transmembrane and cytoplasmic signaling domains. Alvarez-Vallina, L, Curr Gene Ther 1:385-397 (2001). CAR-mediated recognition converts tumor-associated antigens (TAA) expressed on the cell surface into recruitment points of effector functions, addressing the goal of major histocompatibility complex-independent activation of effector cells. First-generation CARs were constructed through the fusion of a scFv-based TAA-binding domain to a cytoplasmic signaling domain typically derived either from the ζ chain of the T cell receptor (TCR)/CD3 complex or from the γ chain associated with some Fc receptors. Gross, G. et al., Proc Natl Acad Sci USA 86:10024-10028 (1989). Second-generation CARs (CARv2) comprising the signaling region of the TCR ζ in series with the signaling domain derived from the T-cell co-stimulatory receptors CD28, 4-IBB (CD137) or OX40 (CD134) have also been developed. Sanz, L. et al., Trends Immunol 25:85-91 (2004). Upon encountering antigen, the interaction of a genetically transferred CAR triggers effector functions and can mediate cytolysis of tumor cells. The utility and effectiveness of the CAR approach have been demonstrated in a variety of animal models, and ongoing clinical trials using CAR-based genetically engineered T lymphocytes for the treatment of cancer patients. Lipowska-Bhalla, G. et al., Cancer Immunol Immunother 61:953-962 (2012). CARs enable targeting of effector cells toward any native extracellular antigen for which a suitable antibody exists. Engineered cells can be targeted not only to proteins but also to structures such as carbohydrate and glycolipid tumor antigens. Mezzanzanica, D. et al., Cancer Gene Ther 5:401-407 (1998); Kershaw, M H. et al., Nat Rev Immunol 5:928-940 (2005).

Current methods for the generation of recombinant antibodies are mainly based on the use of purified proteins. Hoogenboom, H. R. et al., Nat Biotechnol 23:1105-1116 (2005). However, a mammalian cell-based antibody display platform has recently been described, which takes advantage of the functional capabilities of T lymphocytes. Alonso-Camino et al, Molecular Therapy Nucleic Acids (2013) 2, e93. The display of antibodies on the surface of T lymphocytes, as a part of a CAR-mediating signaling, may ideally link the antigen-antibody interaction to a demonstrable change in cell phenotype, due to the surface expression of activation markers. Alonso-Camino, V. et al., PLOS ONE 4: e7174 (2009). By using a scFv-based CAR that recognizes a TAA, it has been demonstrated that combining CAR-mediated activation with fluorescence-activated cell sorting (FACS) of CD69+ T cells makes it possible to isolate binders to surface TAA, with an enrichment factor of at least 10³-fold after two rounds, resulting in a homogeneous population of T cells expressing TAA-specific CAR. Alonso-Camino, V, et al., PLOS ONE 4: e7174 (2009).

### E. Anti-GPC3 Antibody Variants and Modifications

### 1. Variants

In addition to the anti-GPC3 antibodies described herein, it is contemplated that anti-GPC3 antibody variants can be prepared. Anti-GPC3 antibody variants can be prepared by introducing appropriate nucleotide changes into the encoding DNA, and/or by synthesis of the desired antibody or polypeptide. Those skilled in the art will appreciate that amino acid changes may alter post-translational processes of the anti-GPC3 antibody, such as changing the number or position of glycosylation sites or altering the membrane anchoring characteristics.

Variations in the anti-GPC3 antibodies described herein, can be made, for example, using any of the techniques and guidelines for conservative and non-conservative mutations set forth, for instance, in U.S. Pat. No. 5,364,934. Variations may be a substitution, deletion or insertion of one or more codons encoding the antibody or polypeptide that results in a change in the amino acid sequence as compared with the native sequence antibody or polypeptide. Optionally the variation is by substitution of at least one amino acid with any other amino acid in one or more of the domains of the anti-GPC3 antibody. Guidance in determining which amino acid residue may be inserted, substituted or deleted without adversely affecting the desired activity may be found by comparing the sequence of the anti-GPC3 antibody with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Anti-GPC3 antibody fragments are provided herein. Such fragments may be truncated at the N-terminus or C-terminus, or may lack internal residues, for example, when compared with a full length native antibody or protein. Certain fragments lack amino acid residues that are not essential for a desired biological activity of the anti-GPC3 antibody.

Anti-GPC3 antibody fragments may be prepared by any of a number of conventional techniques. Desired peptide fragments may be chemically synthesized. An alternative approach involves generating antibody or polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired antibody or polypeptide fragment, by polymerase chain reaction (PCR). Oligonucleotides that define the desired termini of the DNA fragment are employed at the 5′ and 3′ primers in the PCR. Preferably, anti-GPC3 antibody fragments share at least one biological and/or immunological activity with the native anti-GPC3 antibody disclosed herein.

In particular embodiments, conservative substitutions of interest are shown in Table 1 under the heading of preferred substitutions. If such substitutions result in a change in biological activity, then more substantial changes, denominated exemplary substitutions in Table 1, or as further described below in reference to amino acid classes, are introduced and the products screened.

TABLE 1
Original Exemplary Preferred Residue Substitutions Substitutions

Ala (A) val; leu; ile val
Arg (R) lys; gin; asn lys
Asn (N) gin; his; lys; arg gin
Asp (D) glu glu
Cys (C) ser ser
Gin (Q) asn asn
Glu (E) asp asp
Gly (G) pro; ala ala
His (H) asn; gin; lys; arg arg
He (I) leu; val; met; ala; phe; norleucine leu
Leu (L) norleucine; ile; val; met; ala; phe ile
Lys (K) arg; gin; asn arg
Met (M) leu; phe; ile leu
Phe (F) leu; val; ile; ala; tyr leu
Pro (P) ala ala
Ser (S) thr thr
Thr (T) ser ser
Trp (W) tyr; phe tyr
Tyr (Y) trp; phe; thr; ser phe
Val (V) ile; leu; met; phe; ala; norleucine leu

Substantial modifications in function or immunological identity of the anti-GPC3 antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gin, his, lys, arg; (5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the anti-GPC3 antibody variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244:1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

Any cysteine residue not involved in maintaining the proper conformation of the anti-GPC3 antibody also may be substituted, generally with serine, to improve the oxidative stability of the molecule and prevent aberrant crosslinking. Conversely, cysteine bond(s) may be added to the anti-GPC3 antibody to improve its stability (particularly where the antibody is an antibody fragment such as an Fv fragment).

A particularly preferred type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene Ill product of MI 3 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and GPC3 polypeptide. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the anti-GPC3 antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the anti-GPC3 antibody.

### 2. Modifications

Covalent modifications of anti-GPC3 antibodies are included within the scope of this invention. One type of covalent modification includes reacting targeted amino acid residues of an anti-GPC3 antibody with an organic derivatizing agent that is capable of reacting with selected side chains or the N- or C-terminal residues of the anti-GPC3 antibody. Derivatization with bifunctional agents is useful, for instance, for crosslinking anti-GPC3 antibody to a water-insoluble support matrix or surface for use in the method for purifying anti-GPC3 antibodies, and vice-versa. Commonly used crosslinking agents include, e.g., I,I-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis (succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the anti-GPC3 antibody included within the scope of this invention comprises altering the native glycosylation pattern of the antibody or polypeptide. “Altering the native glycosylation pattern” is intended for purposes herein to mean deleting one or more carbohydrate moieties found in native sequence anti-GPC3 antibody (either by removing the underlying glycosylation site or by deleting the glycosylation by chemical and/or enzymatic means), and/or adding one or more glycosylation sites that are not present in the native sequence anti-GPC3 antibody. In addition, the phrase includes qualitative changes in the glycosylation of the native proteins, involving a change in the nature and proportions of the various carbohydrate moieties present.

Glycosylation of antibodies and other polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the anti-GPC3 antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original anti-GPC3 antibody (for O-linked glycosylation sites). The anti-GPC3 antibody amino acid sequence may optionally be altered through changes at the DNA level, particularly by mutating the DNA encoding the anti-GPC3 antibody at preselected bases such that codons are generated that will translate into the desired amino acids.

Another means of increasing the number of carbohydrate moieties on the anti-GPC3 antibody is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981). Removal of carbohydrate moieties present on the anti-GPC3 antibody may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

### F. Preparation of Anti-GPC3 Antibodies

The description below relates primarily to production of anti-GPC3 antibodies by culturing cells transformed or transfected with a vector containing anti-GPC3 antibody-encoding nucleic acid. It is, of course, contemplated that alternative methods, which are well known in the art, may be employed to prepare anti-GPC3 antibodies. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, CA (1969); Merrifield, J. Am. Chem. Soc, 85:2149-2154 (1963)]. In vitro protein synthesis may be performed using manual techniques or by automation. Automated synthesis may be accomplished, for instance, using an Applied Biosystems Peptide Synthesizer (Foster City, CA) using manufacturer's instructions. Various portions of the anti-GPC3 antibody may be chemically synthesized separately and combined using chemical or enzymatic methods to produce the desired anti-GPC3 antibody.

### 1. Isolation of DNA Encoding Anti-GPC3 Antibody

DNA encoding anti-GPC3 antibody may be obtained from a cDNA library prepared from tissue believed to possess the anti-GPC3 antibody mRNA and to express it at a detectable level. Accordingly, human anti-GPC3 antibody DNA can be conveniently obtained from a cDNA library prepared from human tissue. The anti-GPC3 antibody-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding anti-GPC3 antibody is to use PCR methodology [Sambrook et al., supra; Dieffenbach et al., PCR Primer: A Laboratory Manual (Cold Spring Harbor Laboratory Press, 1995)].

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened.

Methods of labeling are well known in the art, and include the use of radiolabels like 32P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

### 2. Selection and Transformation of Host Cells

Host cells are transfected or transformed with expression or cloning vectors described herein for anti-GPC3 antibody production and cultured in conventional nutrient media modified as appropriate for inducing promoters, selecting trans formants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation, which means introduction of DNA into the host so that the DNA is replicable, either as an extrachromosomal or by chromosomal integrant, are known to the ordinarily skilled artisan, for example, CaC12, CaP04, liposome-mediated, polyethylene-gycol/DMSO and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al, Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bac, 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells.

a. Prokaryotic Host Cells

Suitable prokaryotes include but are not limited to archaebacteria and eubacteria, such as Gram-negative or Gram-positive organisms, for example, Enterobacteriaceae such as E. coli. Various E. coli strains are publicly available, such as E. coli K12 strain MM294 (ATCC 31,446); E. coli XI 776 (ATCC 31,537); E. coli strain W3110 (ATCC 27,325) and K5772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, Rhizobia, Vitreoscilla, Paracoccus and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 (Bachmann, Cellular and Molecular Biology, vol. 2 (Washington, D.C.: American Society for Microbiology, 1987), pp. 1190-1219; ATCC Deposit No. 27,325) may be modified to effect a genetic mutation in the genes encoding proteins endogenous to the host, with examples of such hosts including E. coli W3110 strain 1 A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E 15 (argF-lac) 169 degP ompT kanr; E. coli W3110 strain 37D6, which has the complete genotype tonA ptr3 phoA E15 (argF-lac) 169 degP ompT rbs7 ilvG kanr; E. coli W3110 strain 40B4, which is strain 37D6 with a non-kanamycin resistant degP deletion mutation; E. coli W3110 strain 33D3 having genotype W3110 AfhuA (AtonA) ptr3 lac Iq lacL8 AompTA (nmpc-fepE) degP41 kanR (U.S. Pat. No. 5,639,635) and an E. coli strain having mutant periplasmic protease disclosed in U.S. Pat. No. 4,946,783 issued 7 Aug. 1990. Other strains and derivatives thereof, such as E. coli 294 (ATCC 31,446), E. coli B, üθiiλ 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture. Alternatively, in vitro methods of cloning, e.g., PCR or other nucleic acid polymerase reactions, are suitable.

Full length antibody, antibody fragments, and antibody fusion proteins can be produced in bacteria, in particular when glycosylation and Fc effector function are not needed. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation regio (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed e.g, in CHO cells.

b. Eukaryotic Host Cells

In addition to prokaryotes, eukaryotic microbes such as filamentous fungi or yeast are suitable cloning or expression hosts for anti-GPC3 antibody-encoding vectors. Saccharomyces cerevisiae is a commonly used lower eukaryotic host microorganism. Others include Schizosaccharomyces pombe (Beach and Nurse, Nature, 290:140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacterid., 154 (2): 737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81:1470-1474 [1984]) and A. niger (Kelly and Hynes, EMBO J., 4:475-479 [1985]) Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated anti-GPC3 antibody are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-I variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CVI line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for anti-GPC3 antibody production and cultured in conventional nutrient media modified as appropriate for inducing promoters, selecting transformants, or amplifying the genes encoding the desired sequences.

### 3. Selection and Use of a Replicable Vector

For recombinant production of an antibody of the invention, the nucleic acid (e.g., cDNA or genomic DNA) encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The choice of vector depends in part on the host cell to be used. Generally, preferred host cells are of either prokaryotic or eukaryotic (generally mammalian) origin.

The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. The GPC3 may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the anti-GPC3 antibody-encoding DNA that is inserted into the vector. The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader (including Saccharomyces and Kluyveromyces a-factor leaders, the latter described in U.S. Pat. No. 5,010,182), or acid phosphatase leader, the C. albicans glucoamylase leader (EP 362, 179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

a. Prokaryotic Host Cells

Polynucleotide sequences encoding polypeptide components of the antibody of the invention can be obtained using standard recombinant techniques. Desired polynucleotide sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides.

In general, plasmid vectors containing replicon and control sequences which are derived from species compatible with the host cell are used in connection with these hosts. Both expression and cloning vectors contain a nucleic acid sequence that enables the vector to replicate in one or more selected host cells, as well as marking sequences which are capable of providing phenotypic selection in transformed cells. Such sequences are well known for a variety of bacteria, yeast, and viruses. The origin of replication from the plasmid pBR322, which contains genes encoding ampicillin (Amp) and tetracycline (Tet) resistance and thus provides easy means for identifying transformed cells, is suitable for most Gram-negative bacteria, the 2u plasmid origin is suitable for yeast, and various viral origins (SV40, polyoma, adenovirus, VSV or BPV) are useful for cloning vectors in mammalian cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as AOEM™-11 may be utilized in making a recombinant vector which can be used to transform susceptible host cells such as E. coli LE392.

The expression vector of the invention may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector of the invention. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters recognized by a variety of potential host cells are well known. Promoters suitable for use with prokaryotic hosts include the PhoA promoter, the β-galactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776] and hybrid promoters such as the tac [deBoer et al., Proc. Natl. Acad. Sci. USA, 80:21-25 (1983)] or the trc promoter. Promoters for use in bacterial systems also will contain a Shine-Dalgarno (S.D.) sequence operably linked to the DNA encoding anti-GPC3 antibody. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (Siebenlist et al. (1980) Cell 20:269) using linkers or adaptors to supply any required restriction sites.

In one aspect of the invention, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence is substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP. In one embodiment of the invention, the signal sequences used in both cistrons of the expression system are STII signal sequences or variants thereof.

In another aspect, the production of the immunoglobulins according to the invention can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits. Proba and Pluckthun Gene, 159:203 (1995).

The present invention provides an expression system in which the quantitative ratio of expressed polypeptide components can be modulated in order to maximize the yield of secreted and properly assembled antibodies of the invention. Such modulation is accomplished at least in part by simultaneously modulating translational strengths for the polypeptide components. One technique for modulating translational strength is disclosed in Simmons et al., U.S. Pat. No. 5,840,523. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleotide sequence are preferred. Alterations in the TIR can include, for example, alterations in the number or spacing of Shine-Dalgarno sequences, along with alterations in the signal sequence. One method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al. (1992) METHODS: A Companion to Methods in Enzymol. 4:151-158.

Preferably, a set of vectors is generated with a range of TIR strengths for each cistron therein. This limited set provides a comparison of expression levels of each chain as well as the yield of the desired antibody products under various TIR strength combinations. TIR strengths can be determined by quantifying the expression level of a reporter gene as described in detail in Simmons et al. U.S. Pat. No. 5,840,523. Based on the translational strength comparison, the desired individual TIRs are selected to be combined in the expression vector constructs of the invention,

b. Eukaryotic Host Cells

The vector components generally include, but are not limited to, one or more of the following: a signal sequence, an origin of replication, one or more marker genes, an enhancer element, a promoter, and a transcription termination sequence.

### (1) Signal Sequence Component

A vector for use in a eukaryotic host cell may also contain a signal sequence or other polypeptide having a specific cleavage site at the N-terminus of the mature protein or polypeptide of interest. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

### (2) Origin of Replication

Generally, an origin of replication component is not needed for mammalian expression vectors. For example, the SV40 origin may typically be used only because it contains the early promoter.

### (3) Selection Gene Component

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

An example of suitable selectable markers for mammalian cells are those that enable the identification of cells competent to take up the anti-GPC3 antibody-encoding nucleic acid, such as DHFR or thymidine kinase, metallothionein-I and -II, preferably primate metallothionein genes, adenosine deaminase, ornithine decarboxylase, etc. An appropriate host cell when wild-type DHFR is employed is the CHO cell line deficient in DHFR activity (e.g., ATCC CRL-9096), prepared and propagated as described by Urlaub et al., Proc. Natl. Acad. Sci. USA, 77:4216 (1980). For example, cells transformed with the DHFR selection gene are first identified by culturing all of the transformants in a culture medium that contains methotrexate (Mtx), a competitive antagonist of DHFR. Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trpl gene present in the yeast plasmid YRp7 [Stinchcomb et al., Nature, 282:39 (1979); Kingsman et al., Gene, 7:141 (1979); Tschemper et al., Gene, 10:157 (1980)]. The trpl gene provides a selection marker for a mutant strain of yeast lacking the ability to grow in tryptophan, for example, ATCC No. 44076 or PEP4-1 [Jones, Genetics, 85:12 (1977)].

### (4) Promoter Component Expression

Cloning vectors usually contain a promoter operably linked to the anti-GPC3 antibody-encoding nucleic acid sequence to direct mRNA synthesis. Promoters recognized by a variety of potential host cells are well known.

Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Anti-GPC3 antibody transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human-interferon cDNA in mouse cells under the control of a thymidine kinase promoter from herpes simplex virus. Alternatively, the Rous Sarcoma Virus long terminal repeat can be used as the promoter.

### (5) Enhancer Element Component

Transcription of a DNA encoding the anti-GPC3 antibody by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, a-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the anti-GPC3 antibody coding sequence, but is preferably located at a site 5′ from the promoter.

### (6) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding anti-GPC3 antibody. One useful transcription termination component is the bovine growth hormone polyadenylation region. See WO94/11026 and the expression vector disclosed therein.

Still other methods, vectors, and host cells suitable for adaptation to the synthesis of anti-GPC3 antibody in recombinant vertebrate cell culture are described in Gething et al., Nature, 293:620-625 (1981); Mantei et al., Nature, 281:40-46 (1979); EP 117,060; and EP 117,058.

### 4. Culturing the Host Cells

The host cells used to produce the anti-GPC3 antibody of this invention may be cultured in a variety of media.

a. Prokaryotic Host Cells

Prokaryotic cells used to produce the polypeptides of the invention are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol.

The prokaryotic host cells are cultured at suitable temperatures. For E. coli growth, for example, the preferred temperature ranges from about 20° C. to about 39° C., more preferably from about 25° C. to about 37° C., even more preferably at about 30° C. The pH of the medium may be any pH ranging from about 5 to about 9, depending mainly on the host organism. For E. coli, the pH is preferably from about 6.8 to about 7.4, and more preferably about 7.0.

If an inducible promoter is used in the expression vector of the invention, protein expression is induced under conditions suitable for the activation of the promoter. In one aspect of the invention, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods (2002), 263:133-147). A variety of other inducers may be used, according to the vector construct employed, as is known in the art.

In one embodiment, the expressed polypeptides of the present invention are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

In one aspect of the invention, antibody production is conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (the preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides of the invention, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis, trans-isomerase with chaperone activity) can be used to co-transform the host prokaryotic cells. The chaperone proteins have been demonstrated to facilitate the proper folding and solubility of heterologous proteins produced in bacterial host cells. Chen et al. (1999) J Bio Chem 274:19601-19605; Georgiou et al., U.S. Pat. No. 6,083,715; Georgiou et al., U.S. Pat. No. 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210.

To minimize proteolysis of expressed heterologous proteins (especially those that are proteolytically sensitive), certain host strains deficient for proteolytic enzymes can be used for the present invention. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available and described in, for example, Joly et al. (1998), supra; Georgiou et al., U.S. Pat. No. 5,264,365; Georgiou et al., U.S. Pat. No. 5,508,192; Hara et al., Microbial Drug Resistance, 2:63-72 (1996).

In one embodiment, E. coli strains deficient for proteolytic enzymes and transformed with plasmids overexpressing one or more chaperone proteins are used as host cells in the expression system of the invention.

b. Eukaryotic Host Cells

Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### 5. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected. Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence GPC3 polypeptide or against a synthetic peptide based on the DNA sequences provided herein or against exogenous sequence fused to GPC3 DNA and encoding a specific antibody epitope.

### 6. Purification of Anti-GPC3 Antibody

Forms of anti-GPC3 antibody may be recovered from culture medium or from host cell lysates. If membrane-bound, it can be released from the membrane using a suitable detergent solution (e.g. Triton-X 100) or by enzymatic cleavage. Cells employed in expression of anti-GPC3 antibody can be disrupted by various physical or chemical means, such as freeze-thaw cycling, sonication, mechanical disruption, or cell lysing agents.

It may be desired to purify anti-GPC3 antibody from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the anti-GPC3 antibody.

Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular anti-GPC3 antibody produced.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supematants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γï, γ2 or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly (styrenedivinyl) benzene allow for faster flow rates and shorter processing times than can be achieved with agarose. Where the antibody comprises a CH3 domain, the Bakerbond ABX™resin (J. T. Baker, Phillipsburg, NJ) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### G. Pharmaceutical Formulations

The antibodies of the invention may be administered by any route appropriate to the condition to be treated. The antibody will typically be administered parenterally, i.e. infusion, subcutaneous, intramuscular, intravenous, intradermal, intrathecal and epidural.

For treating these cancers, in one embodiment, the antibody is administered via intravenous infusion. The dosage administered via infusion is in the range of about 1 μg/m² to about 10,000 μg/m² per dose, generally one dose per week for a total of one, two, three or four doses. Alternatively, the dosage range is of about 1 μg/m² to about 1000 μg/m², about 1 μg/m² to about 800 μg/m², about 1 μg/m² to about 600 μg/m², about 1 μg/m² to about 400 μg/m², about 10 μg/m² to about 500 μg/m², about 10 μg/m² to about 300 μg/m², about 10 μg/m² to about 200 μg/m², and about 1 μg/m² to about 200 μg/m². The dose may be administered once per day, once per week, multiple times per week, but less than once per day, multiple times per month but less than once per day, multiple times per month but less than once per week, once per month or intermittently to relieve or alleviate symptoms of the disease. Administration may continue at any of the disclosed intervals until remission of the tumor or symptoms of the cancer being treated. Administration may continue after remission or relief of symptoms is achieved where such remission or relief is prolonged by such continued administration.

The invention also provides a method of treating breast cancer comprising administering to a patient suffering from breast cancer, a therapeutically effective amount of a humanized GPC3 antibody of any one of the preceding embodiments. The antibody will typically be administered in a dosage range of about 1 μg/m² to about 1000 mg/m².

In one aspect, the invention further provides pharmaceutical formulations comprising at least one anti-GPC3 antibody of the invention. In some embodiments, a pharmaceutical formulation comprises (1) an antibody of the invention, and (2) a pharmaceutically acceptable carrier.

Therapeutic formulations comprising an anti-GPC3 antibody used in accordance with the present invention are prepared for storage by mixing the antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG). Pharmaceutical formulations to be used for in vivo administration are generally sterile. This is readily accomplished by filtration through sterile filtration membranes.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

Sustained-release preparations may be prepared. Suitable examples of sustained-release preparations include semi-permeable matrices of solid hydrophobic polymers containing the antibody, which matrices are in the form of shaped articles, e.g., films, or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

An antibody may be formulated in any suitable form for delivery to a target cell/tissue. For example, antibodies may be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidyl ethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-288 (1982) via a disulfide interchange reaction. A chemotherapeutic agent is optionally contained within the liposome. See Gabizon et al., J. National Cancer Inst. 81 (19): 1484 (1989).

The formulations to be used for in vivo administration must be sterile. This is readily accomplished by filtration through sterile filtration membranes.

H. Treatment with Anti-GPC3 Antibodies

To determine GPC3 expression in a cancer, various detection assays are available. In one embodiment, GPC3 polypeptide overexpression may be analyzed by immunohistochemistry (IHC). Parrafin embedded tissue sections from a tumor biopsy may be subjected to the IHC assay and accorded a GPC3 protein staining intensity criteria. In a preferred embodiment, determining whether a cancer is amenable to treatment by methods disclosed herein involves detecting the presence of the GPC3 tumor epitope in a subject or in a sample from a subject.

Alternatively, or additionally, FISH assays such as the INFORM® (sold by Ventana, Arizona) or PATHVISION® (Vysis, Illinois) may be carried out on formalin-fixed, paraffin-embedded tumor tissue to determine the extent (if any) of GPC3 overexpression in the tumor.

GPC3 overexpression or amplification may be evaluated using an in vivo detection assay, e.g., by administering a molecule (such as an antibody) which binds the molecule to be detected and is tagged with a detectable label (e.g., a radioactive isotope or a fluorescent label) and externally scanning the patient for localization of the label. As described above, the anti-GPC3 antibodies of the invention have various non-therapeutic applications. The anti-GPC3 antibodies of the present invention can be useful for staging of GPC3 epitope-expressing cancers (e.g., in radioimaging). The antibodies are also useful for purification or immunoprecipitation of GPC3 epitope from cells, for detection and quantitation of GPC3 epitope in vitro, e.g., in an ELISA or a Western blot, to kill and eliminate GPC3-expressing cells from a population of mixed cells as a step in the purification of other cells.

Currently, depending on the stage of the cancer, cancer treatment involves one or a combination of the following therapies: surgery to remove the cancerous tissue, radiation therapy, and chemotherapy. Anti-GPC3 antibody therapy may be especially desirable in elderly patients who do not tolerate the toxicity and side effects of chemotherapy well and in metastatic disease where radiation therapy has limited usefulness. The tumor targeting anti-GPC3 antibodies of the invention are useful to alleviate GPC3-expressing cancers upon initial diagnosis of the disease or during relapse.

The anti-GPC3 antibodies are administered to a human patient, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous or subcutaneous administration of the antibody is preferred.

The antibody composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

For the prevention or treatment of disease, the dosage and mode of administration will be chosen by the physician according to known criteria. The appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Preferably, the antibody is administered by intravenous infusion or by subcutaneous injections. Depending on the type and severity of the disease, about 1 μg/kg to about 50 mg kg body weight (e.g., about 0.1-15 mg/kg/dose) of antibody can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the anti-GPC3 antibody. However, other dosage regimens may be useful. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

The anti-GPC3 antibodies of the invention can be in the different forms encompassed by the definition of “antibody” herein. Thus, the antibodies include full length or intact antibody, antibody fragments, native sequence antibody or amino acid variants, humanized, chimeric or fusion antibodies, and functional fragments thereof. In fusion antibodies an antibody sequence is fused to a heterologous polypeptide sequence. The antibodies can be modified in the Fc region to provide desired effector functions. As discussed in more detail in the sections herein, with the appropriate Fc regions, the naked antibody bound on the cell surface can induce cytotoxicity, e.g., via antibody-dependent cellular cytotoxicity (ADCC) or by recruiting complement in complement dependent cytotoxicity, or some other mechanism. Alternatively, where it is desirable to eliminate or reduce effector function, so as to minimize side effects or therapeutic complications, certain other Fc regions may be used.

In one embodiment, the antibody (i) competes for binding to the same epitope, and/or (ii) binds substantially to the same epitope, as the antibodies of the invention. Antibodies having the biological characteristics of the present anti-GPC3 antibodies of the invention are also contemplated, specifically including the in vivo tumor targeting and any cell proliferation inhibition or cytotoxic characteristics.

The present anti-GPC3 antibodies are useful for treating a GPC3-expressing cancer or alleviating one or more symptoms of the cancer in a mammal. The cancers encompass metastatic cancers of any of the cancers described herein. The antibody is able to bind to at least a portion of the cancer cells that express GPC3 epitope in the mammal. In a preferred embodiment, the antibody is effective to destroy or kill GPC3-expressing tumor cells or inhibit the growth of such tumor cells, in vitro or in vivo, upon binding to GPC3 epitope on the cell. In other preferred embodiments, the antibodies are effective to (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind.

The invention provides a composition comprising an anti-GPC3 antibody of the invention, and a carrier. The invention also provides formulations comprising an anti-GPC3 antibody of the invention, and a carrier. In one embodiment, the formulation is a therapeutic formulation comprising a pharmaceutically acceptable carrier.

Another aspect of the invention is isolated nucleic acids encoding the anti-GPC3 antibodies. Nucleic acids encoding both the H and L chains and especially the hypervariable region residues, chains which encode the native sequence antibody as well as variants, modifications and humanized versions of the antibody, are encompassed.

The invention also provides methods useful for treating a GPC3 polypeptide-expressing cancer or alleviating one or more symptoms of the cancer in a mammal, comprising administering a therapeutically effective amount of an anti-GPC3 antibody to the mammal. The antibody therapeutic compositions can be administered short term (acute) or chronic, or intermittent as directed by physician. Also provided are methods of inhibiting the growth of, and killing a GPC3 polypeptide-expressing cell.

The invention also provides kits and articles of manufacture comprising at least one anti-GPC3 antibody. Kits containing anti-GPC3 antibodies find use, e.g., for GPC3 cell killing assays, for purification or immunoprecipitation of GPC3 polypeptide from cells. For example, for isolation and purification of GPC3, the kit can contain an anti-GPC3 antibody coupled to beads (e.g., sepharose beads). Kits can be provided which contain the antibodies for detection and quantitation of GPC3 in vitro, e.g., in an ELISA or a Western blot or an IHC assay (described in greater detail herein). Such antibody useful for detection may be provided with a label such as a fluorescent or radiolabel.

### Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody.

Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgGi, IgG2, IgG3, or IgG4) that is responsible for increasing the in vivo serum half-life of the IgG molecule.

Treatment with CAR Modified Immune Cells

In certain embodiments, the invention relates to compositions and methods for treating cancer including but not limited to hematologic malignancies and solid tumors. In certain embodiments, CAR modified immune cells are used. CAR-T cells can be used therapeutically for patients suffering from non-hematological tumors such as solid tumors arising from breast, CNS, and skin malignancies. In certain embodiments, CAR-NK cells can be used therapeutically for patients suffering from any one of a number of malignancies. In certain embodiments, the present invention relates to a strategy of adoptive cell transfer of T cells or NK cells transduced to express a chimeric antigen receptor (CAR). CARs are molecules that combine antibody-based specificity for a desired antigen (e.g., tumor antigen) with, for example, a T cell receptor-activating intracellular domain to generate a chimeric protein that exhibits a specific anti-tumor cellular immune activity.

In one aspect, the present invention relates to the use of NK cells genetically modified to stably express a desired CAR. NK cells expressing a CAR are referred to herein as CAR-NK cells or CAR modified NK cells. Preferably, the cell can be genetically modified to stably express an antibody binding domain on its surface, conferring novel antigen specificity. Methods for generating CAR-NK cells are known in the art. For example, see Glienke et al., Advantages and applications of CAR-expressing natural killer cells, Front Pharmacol. 2015; 6:21. Services for generating CAR-NK cells are commercially available. See for example Creative Biolabs Inc., 45-1 Ramsey Road, Shirley, NY 11967, USA.

In one aspect, the present invention relates to the use of T cells genetically modified to stably express a desired CAR. T cells expressing a CAR are referred to herein as CAR-T cells or CAR modified T cells. Preferably, the cell can be genetically modified to stably express an antibody binding domain on its surface, conferring novel antigen specificity that is MHC independent. In some instances, the T cell is genetically modified to stably express a CAR that combines an antigen recognition domain of a specific antibody with an intracellular domain of the CD3-zeta chain or FcγRI protein into a single chimeric protein.

In one embodiment, the CAR of the invention comprises an extracellular domain having an antigen recognition domain, a transmembrane domain, and a cytoplasmic domain. In one embodiment, the transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex. In one embodiment, the transmembrane domain is the CD8a hinge domain.

With respect to the cytoplasmic domain, the CAR of the invention can be designed to comprise the CD28 and/or 4-IBB signaling domain by itself or be combined with any other desired cytoplasmic domain(s) useful in the context of the CAR of the invention. In one embodiment, the cytoplasmic domain of the CAR can be designed to further comprise the signaling domain of CD3-zeta. For example, the cytoplasmic domain of the CAR can include but is not limited to CD3-zeta, 4-1BB and CD28 signaling modules and combinations thereof. Accordingly, the invention provides CAR T cells and methods of their use for adoptive therapy.

In one embodiment, the CAR T cells of the invention can be generated by introducing a lentiviral vector comprising a desired CAR, for example a CAR comprising anti-GPC3, CD8a hinge and transmembrane domain, and human 4-1BB and CD3zeta signaling domains, into the cells. The CAR T cells of the invention are able to replicate in vivo resulting in long-term persistence that can lead to sustained tumor control.

In one embodiment, the anti-GPC3 domain comprises a heavy chain variable region comprising:

EVQLQQSGPELVKPGASVKISCKTSGYTFTEYAMHWVKQSHGKSLEWIG
GINPNNGVTTYNQRFKGKATLTVDKSSSTAYMELRSLTSEDSAVYYCAR
GLLWYAYWGQGTLVTVSA

In one embodiment, the anti-GPC3 domain comprises a light chain variable region comprising:

(SEQ ID NO: 4)
DIKMTQSPSSMYASLGERVTITCKASQDINSYLSWFQQKPGKSPKTLIY
RANRLVDGVPSRFSGSGSGQDYSLTISSLEYEDMGIYYCLQYDEFPLTF
GAGTKLELK

In one embodiment, the anti-GPC3 domain comprises SEQ ID NO:2 and SEQ ID NO: 4.

In one embodiment, the anti-GPC3 domain comprises an amino acid sequence selected from the group consisting of: EYAMH (SEQ ID NO:6); GINPNNGVTTYNQRFKG (SEQ ID NO: 8); and GLLWYAY (SEQ ID NO: 10).

In one embodiment, the anti-GPC3 domain comprises an amino acid sequence selected from the group consisting of: KASQDINSYLS (SEQ ID NO: 13); RANRLVD (SEQ ID NO: 15); and LQYDEFPLT (SEQ ID NO:17).

In one embodiment, the anti-GPC3 domain comprises an amino acid sequence selected from the group consisting of: SEQ ID NOs: 6, 8, 10; and further comprises an amino acid sequence selected from the group consisting of SEQ ID NOs: 13, 15, 17;

In one embodiment the invention relates to administering a genetically modified T cell expressing a CAR for the treatment of a patient having cancer or at risk of having cancer using lymphocyte infusion. Preferably, autologous lymphocyte infusion is used in the treatment. Autologous PBMCs are collected from a patient in need of treatment and T cells are activated and expanded using the methods described herein and known in the art and then infused back into the patient.

The invention also includes treating a malignancy or an autoimmune disease in which chemotherapy and/or immunotherapy in a patient results in significant immunosuppression in the patient, thereby increasing the risk of the patient of developing a malignancy (e.g., CLL). The invention includes using T cells expressing an anti-GPC3 antibody derived CAR including both CD3-zeta and either the 4-IBB or CD28 costimulatory domain (also referred to as CARTGPC3 T cells). The CARTGPC3 T cells of the invention can undergo robust in vivo T cell expansion and can establish memory cells specific for cells displaying GPC3 tumor epitope, which memory cells persist at high levels for an extended amount of time in blood and bone marrow. The present invention provides chimeric antigen receptor (CAR) comprising an extracellular and intracellular domain. The extracellular domain comprises a target-specific binding element otherwise referred to as an antigen binding moiety. The intracellular domain or otherwise the cytoplasmic domain comprises, a costimulatory signaling region and a zeta chain portion. The costimulatory signaling region refers to a portion of the CAR comprising the intracellular domain of a costimulatory molecule. Costimulatory molecules are cell surface molecules other than antigens receptors or their ligands that are required for an efficient response of lymphocytes to antigen.

Between the extracellular domain and the transmembrane domain of the CAR, or between the cytoplasmic domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

### Antigen Binding Moiety

In one embodiment, the CAR of the invention comprises a target-specific binding element otherwise referred to as an antigen binding moiety, or targeting arm. Antigen binding moieties used in the present invention are capable of binding the GPC3 polypeptide expressed on the surface of cancer cells. As such, the antigen binding moiety is chosen to recognize a ligand that acts as a cell surface marker on target cells associated with a particular disease state.

A CAR of the invention is engineered to target a cell GPC3 by way of engineering an appropriate antigen binding moiety that specifically binds to an epitope of GPC3.

Preferably, the antigen binding moiety portion in the CAR of the invention is scFV, or scFab wherein the nucleic acid sequence of the scFV comprises the nucleic acid sequence(s) of one or more light chain CDRs and one or more heavy chain CDRs disclosed herein for anti-GPC3 antibodies, and wherein the nucleic acid sequence of the scFab comprises the nucleic acid sequence(s) of one or more light chain CDRs and one or more heavy chain CDRs disclosed herein for anti-GPC3 antibodies.

Preferably, the antigen binding moiety portion in the CAR of the invention is an scFV, or scFab comprising an amino acid sequence selected from the group consisting of: EYAMH (SEQ ID NO:6); GINPNNGVTTYNQRFKG (SEQ ID NO:8); and GLLWYAY (SEQ ID NO:10). Preferably, the antigen binding moiety portion in the CAR of the invention is an scFV, or scFab comprising an amino acid sequence selected from the group consisting of: KASQDINSYLS (SEQ ID NO:13); RANRLVD (SEQ ID NO:15); and LQYDEFPLT (SEQ ID NO:17).

Preferably, the antigen binding moiety portion in the CAR of the invention is an scFV, or scFab comprising an amino acid sequence selected from the group consisting of: SEQ ID NOs: 6, 8, 10; and further comprises an amino acid sequence selected from the group consisting of SEQ ID NOs: 13, 15, 17; and further comprises an amino acid sequence selected from the group consisting of SEQ ID NOs: 2 and 4.

In one embodiment, the antigen binding moiety portion in the CAR of the invention is an scFV, or scFab comprising an amino acid sequence having about 80%, 85%, 90%, or 95% identity to the SEQ ID NOs recited above.

### Transmembrane Domain

With respect to the transmembrane domain, the CAR can be designed to comprise a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain.

Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker. Preferably, the transmembrane domain in the CAR of the invention is the CD8 transmembrane domain. In one embodiment, the CD8 transmembrane domain comprises the nucleic acid sequence of SEQ ID NO: 16 of U.S. Pat. No. 9,102,760. In one embodiment, the CD8 transmembrane domain comprises the nucleic acid sequence that encodes the amino acid sequence of SEQ ID NO: 22 of U.S. Pat. No. 9,102,760. In another embodiment, the CD8 transmembrane domain comprises the amino acid sequence of SEQ ID NO: 22 of U.S. Pat. No. 9,102,760. In another embodiment, sequences disclosed in Table 2 of WO 2017/054089 are used.

In some instances, the transmembrane domain of the CAR of the invention comprises the CD8a hinge domain. In one embodiment, the CD8 hinge domain comprises the nucleic acid sequence of SEQ ID NO: 15 of U.S. Pat. No. 9,102,760. In one embodiment, the CD8 hinge domain comprises the nucleic acid sequence that encodes the amino acid sequence of SEQ ID NO: 21 of U.S. Pat. No. 9,102,760. In another embodiment, the CD8 hinge domain comprises the amino acid sequence of SEQ ID NO: 21 of U.S. Pat. No. 9,102,760. In another embodiment, sequences disclosed in Table 2 of WO 2017/054089 are used.

### Cytoplasmic Domain

The cytoplasmic domain or otherwise the intracellular signaling domain of the CAR of the invention is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

In a preferred embodiment, the cytoplasmic domain of the CAR can be designed to comprise the CD3-zeta signaling domain by itself or combined with any other desired cytoplasmic domain(s) useful in the context of the CAR of the invention. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

The cytoplasmic signaling sequences within the cytoplasmic signaling portion of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain is designed to comprise the signaling domain of CD3-zeta and the signaling domain of CD28. In another embodiment, the cytoplasmic domain is designed to comprise the signaling domain of CD3-zeta and the signaling domain of 4-IBB. In yet another embodiment, the cytoplasmic domain is designed to comprise the signaling domain of CD3-zeta and the signaling domain of CD28 and 4-1BB.

In one embodiment, the cytoplasmic domain in the CAR of the invention is designed to comprise the signaling domain of 4-IBB and the signaling domain of CD3-zeta, wherein the signaling domain of 4-IBB comprises the nucleic acid sequence set forth in SEQ ID NO: 17 of U.S. Pat. No. 9,102,760 and the signaling domain of CD3-zeta comprises the nucleic acid sequence set forth in SEQ ID NO: 18 of U.S. Pat. No. 9,102,760. In another embodiment, sequences disclosed in Table 2 of WO 2017/054089 are used. In one embodiment, the cytoplasmic domain in the CAR of the invention is designed to comprise the signaling domain of 4-IBB and the signaling domain of CD3-zeta, wherein the signaling domain of 4-IBB comprises the nucleic acid sequence that encodes the amino acid sequence of SEQ ID NO: 23 of U.S. Pat. No. 9,102,760 and the signaling domain of CD3-zeta comprises the nucleic acid sequence that encodes the amino acid sequence of SEQ ID NO: 24 of U.S. Pat. No. 9,102,760. In another embodiment, sequences disclosed herein in Table 2 of WO 2017/054089 are used.

In one embodiment, the cytoplasmic domain in the CAR of the invention is designed to comprise the signaling domain of 4-IBB and the signaling domain of CD3-zeta, wherein the signaling domain of 4-IBB comprises the amino acid sequence set forth in SEQ ID NO: 23 of U.S. Pat. No. 9,102,760 and the signaling domain of CD3-zeta comprises the amino acid sequence set forth in SEQ ID NO: 24 of U.S. Pat. No. 9,102,760. In another embodiment, sequences disclosed herein in Table 2 of WO 2017/054089 are used.

### Vectors

The present invention encompasses a DNA construct comprising sequences of a CAR, wherein the sequence comprises the nucleic acid sequence of an antigen binding moiety operably linked to the nucleic acid sequence of an intracellular domain. An exemplary intracellular domain that can be used in the CAR of the invention includes but is not limited to the intracellular domain of CD3-zeta, CD28, 4-1BB, and the like. In some instances, the CAR can comprise any combination of CD3-zeta, CD28, 4-1BB, and the like.

In one embodiment, the CAR of the invention comprises an anti-GPC3 antibody derived scFv, human CD8 hinge and transmembrane domain, and human 4-IBB and CD3zeta signaling domains.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

In addition to the methods described above, the following methods may be used. The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-Ia (EF-Ia). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means. Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20. degree. C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Sources of T Cells

Prior to expansion and genetic modification of the T cells of the invention, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CDI Ib, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28− negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8+ T cells in dilute concentrations. In one embodiment, the concentration of cells used is 5×I0⁶/ml. In other embodiments, the concentration used can be from about I×10⁵/ml to I×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, Cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun 73:316-321, 1991; Bierer et al., Curr. Opin. Immun 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

Whether prior to or after genetic modification of the T cells to express a desirable CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and 7,572,631. In some embodiments, methods and compositions for ex vivo expansion of T cells (e.g., γδ T cells) include, without limitation, those described in WO 2016/081518, WO 2017/197347, WO 2019/099744, and WO 2020/117862, the contents of each of which are incorporated by reference herein in their entirety.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30 (8): 3975-3977, 1998; Haanen et al., J. Exp. Med. 190 (9): 13191328, 1999; Garland et al., J. Immunol Meth. 227 (1-2): 53-63, 1999).

In certain embodiments where the T cells of the invention comprise γδ T cells, the γδ T cells may be selectively expanded according to methods and compositions described in WO 2017/197347.

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle: cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used.

For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8⁺ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGF, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus

T cells that have been exposed to varied stimulation times may exhibit different characteristics. For example, typical blood or apheresed peripheral blood mononuclear cell products have a helper T cell population (TH, CD4⁺) that is greater than the cytotoxic or suppressor T cell population (Tc, CD8⁺). Ex vivo expansion of T cells by stimulating CD3 and CD28 receptors produces a population of T cells that prior to about days 8-9 consists predominately of TH cells, while after about days 8-9, the population of T cells comprises an increasingly greater population of Tc cells. Accordingly, depending on the purpose of treatment, infusing a subject with a T cell population comprising predominately of TH cells may be advantageous. Similarly, if an antigen-specific subset of Tc cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

### Therapeutic Application

The present invention encompasses a cell (e.g., T cell) transduced with a lentiviral vector (LV). For example, the LV encodes a CAR that combines an antigen recognition domain of a specific antibody (e.g., GPC3) with an intracellular domain of CD3-zeta, CD28, 4-1BB, or any combinations thereof. Therefore, in some instances, the transduced T cell can elicit a CAR-mediated T-cell response.

The invention provides the use of a CAR to redirect the specificity of a primary T cell to a tumor antigen. Thus, the present invention also provides a method for stimulating a T cell-mediated immune response to a target cell population or tissue in a mammal comprising the step of administering to the mammal a T cell that expresses a CAR, wherein the CAR comprises a binding moiety that specifically interacts with a predetermined target (e.g., GPC3), a zeta chain portion comprising for example the intracellular domain of human CD3zeta, and a costimulatory signaling region.

In one embodiment, the present invention includes a type of cellular therapy where T cells are genetically modified to express a CAR and the CAR T cell is infused to a recipient in need thereof. The infused cell is able to kill tumor cells in the recipient. Unlike antibody therapies, CAR T cells are able to replicate in vivo resulting in long-term persistence that can lead to sustained tumor control.

In one embodiment, the CAR T cells of the invention can undergo robust in vivo T cell expansion and can persist for an extended amount of time. In another embodiment, the CAR T cells of the invention evolve into specific memory T cells that can be reactivated to inhibit any additional tumor formation or growth.

Without wishing to be bound by any particular theory, the anti-tumor immunity response elicited by the CAR-modified T cells may be an active or a passive immune response. In addition, the CAR mediated immune response may be part of an adoptive immunotherapy approach in which CAR-modified T cells induce an immune response specific to the antigen binding moiety in the CAR.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. In certain embodiments, CAR T cells can be used therapeutically for patients suffering from non-hematological tumors such as solid tumors arising from breast, CNS, and skin malignancies. Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblasts, promyelocyte, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplasia syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In one aspect, CAR T cells may be used for ex vivo immunization. With respect to ex vivo immunization, at least one of the following occurs in vitro prior to administering the cell into a mammal: i) expansion of the cells, ii) introducing a nucleic acid encoding a CAR to the cells, and/or iii) cryopreservation of the cells.

Ex vivo procedures are well known in the art and are discussed more fully below. Briefly, cells are isolated from a mammal (preferably a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells. Ex vivo expansion of specific subpopulations of γδ T cells is also within the scope of this disclosure, the methods and composition of which are described in WO 2017/197347, incorporated herein by reference.

In addition to using a cell-based vaccine in terms of ex vivo immunization, the present invention also provides compositions and methods for in vivo immunization to elicit an immune response directed against an antigen in a patient.

The CAR-modified T cells of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319: 1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells. The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

In certain embodiments of the present invention, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun 73:316-321, 1991; Bierer et al., Curr. Opin. Immun 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. In certain embodiments, 1 to 10 mg per day is used. In other embodiments, larger doses of up to 40 mg per day may be used (for example as described in U.S. Pat. No. 6,120,766).

### Immunoconjugates

The invention also pertains to immunoconjugates (interchangeably referred to as “antibody-drug conjugates,” or “ADCs”) comprising an antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate).

In certain embodiments, an immunoconjugate comprises an antibody and a chemotherapeutic agent or other toxin. Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include 212Bi, 1311, 131 In, 90Y, and 186Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science. 238:1098 (1987). Carbon-14-labeled I-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, auristatin peptides, such as monomethylauristatin (MMAE) (synthetic analog of dolastatin), maytansinoids, such as DM1, a trichothene, and CC1065, and the derivatives of these toxins that have toxin activity, are also contemplated herein. Additional non-limiting examples of toxins include those described in WO 2014144871, the disclosure of which is herein incorporated by reference in its entirety.

### Exemplary Immunoconjugates-Antibody-Drug Conjugates

An immunoconjugate (or “antibody-drug conjugate” (“ADC”)) of the invention may be of Formula I, below, wherein an antibody is conjugated (i.e., covalently attached) to one or more drug moieties (D) through an optional linker (L). ADCs may include thioMAb drug conjugates (“TDC”).

Ab-(L-D)ₚ I

Accordingly, the antibody may be conjugated to the drug either directly or via a linker. In Formula I, p is the average number of drug moieties per antibody, which can range, e.g., from about 1 to about 20 drug moieties per antibody, and in certain embodiments, from 1 to about 8 drug moieties per antibody. The invention includes a composition comprising a mixture of antibody-drug compounds of Formula I where the average drug loading per antibody is about 2 to about 5, or about 3 to about 4.

a. Exemplary Linkers

A linker may comprise one or more linker components. Exemplary linker components include 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), and those resulting from conjugation with linker reagents: N-Succinimidyl 4-(2-pyridylthio) pentanoate forming linker moiety 4-mercaptopentanoic acid (“SPP”), N-succinimidyl 4-(N-maleimidomethyl) cyclohexane-1 carboxylate forming linker moiety 4-((2,5-dioxopyrrolidin-I-yl) methyl) cyclohexanecarboxylic acid (“SMCC”, also referred to herein as “MCC”), 2,5-dioxopyrrolidin-I-yl 4-(pyridin-2-yldisulfanyl) butanoate forming linker moiety 4-mercaptobutanoic acid (“SPDB”), N-Succinimidyl (4-iodo-acetyl) aminobenzoate (“SIAB”), ethyleneoxy-CH2CH2O— as one or more repeating units (“EO” or “PEO”). Additional linker components are known in the art and some are described herein. Various linker components are known in the art, some of which are described below.

A linker may be a “cleavable linker,” facilitating release of a drug in the cell. For example, an acid-labile linker (e.g., hydrazone), protease-sensitive (e.g., peptidase-sensitive) linker, photolabile linker, dimethyl linker or disulfide-containing linker (Chari et al., Cancer Research 52:127-131 (1992); U.S. Pat. No. 5,208,020) may be used.

In certain embodiments, a linker is as shown in the following Formula II:

Aₐ-W_{w}-Y_{y}- II

wherein A is a stretcher unit, and a is an integer from 0 to 1; W is an amino acid unit, and w is an integer from 0 to 12; Y is a spacer unit, and y is 0, 1, or 2; and Ab, D, and p are defined as above for Formula I. Exemplary embodiments of such linkers are described in US 2005-0238649 A1, which is expressly incorporated herein by reference.

In some embodiments, a linker component may comprise a “stretcher unit” that links an antibody to another linker component or to a drug moiety. Exemplary stretcher units are shown below (wherein the wavy line indicates sites of covalent attachment to an antibody):

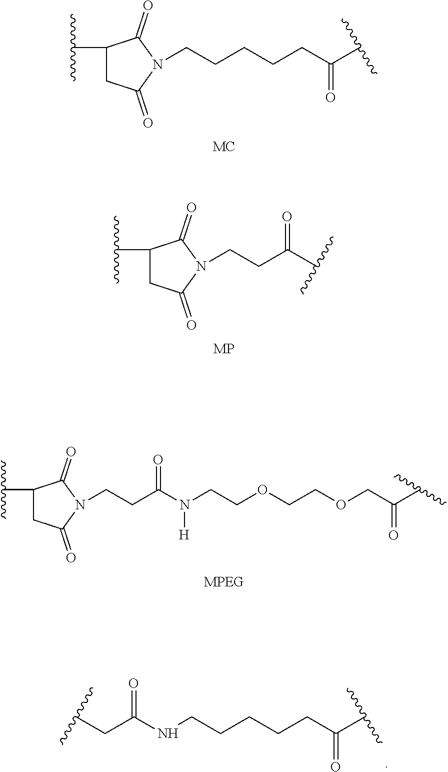 [SMILES: *C(=O)CCCCCN1C(=O)CC(*)C1=O ; *C(=O)CCN1C(=O)CC(*)C1=O ; *C(=O)COCCOCCNC(=O)CCN1C(=O)CC(*)C1=O ; *CC(=O)NCCCCCC(*)=O]

In some embodiments, a linker component may comprise an amino acid unit. In one such embodiment, the amino acid unit allows for cleavage of the linker by a protease, thereby facilitating release of the drug from the immunoconjugate upon exposure to intracellular proteases, such as lysosomal enzymes. See, e.g., Doronina et al. (2003) Nat. Biotechnol. 21:778-784. Exemplary amino acid units include, but are not limited to, a dipeptide, a tripeptide, a tetrapeptide, and a pentapeptide. Exemplary dipeptides include: valine-citrulline (vc or val-cit), alanine-phenylalanine (af or ala-phe); phenylalanine-lysine (fk or phe-lys); or N-methyl-valine-citrulline (Me-val-cit). Exemplary tripeptides include: glycine-valine-citrulline (gly-val-cit) and glycine-glycine-glycine (gly-gly-gly). An amino acid unit may comprise amino acid residues that occur naturally, as well as minor amino acids and non-naturally occurring amino acid analogs, such as citrulline. Amino acid units can be designed and optimized in their selectivity for enzymatic cleavage by a particular enzyme, for example, a tumor-associated protease, cathepsin B, C and D, or a plasmin protease.

In some embodiments, a linker component may comprise a “spacer” unit that links the antibody to a drug moiety, either directly or by way of a stretcher unit and/or an amino acid unit. A spacer unit may be “self-immolative” or a “non-self-immolative.” A “non-self-immolative” spacer unit is one in which part or all of the spacer unit remains bound to the drug moiety upon enzymatic (e.g., proteolytic) cleavage of the ADC. Examples of non-self-immolative spacer units include, but are not limited to, a glycine spacer unit and a glycine-glycine spacer unit. Other combinations of peptidic spacers susceptible to sequence-specific enzymatic cleavage are also contemplated. For example, enzymatic cleavage of an ADC containing a glycine-glycine spacer unit by a tumor-cell associated protease would result in release of a glycine-glycine-drug moiety from the remainder of the ADC. In one such embodiment, the glycine-glycine-drug moiety is then subjected to a separate hydrolysis step in the tumor cell, thus cleaving the glycine-glycine spacer unit from the drug moiety.

A “self-immolative” spacer unit allows for release of the drug moiety without a separate hydrolysis step. In certain embodiments, a spacer unit of a linker comprises a p-aminobenzyl unit. In one such embodiment, a p-aminobenzyl alcohol is attached to an amino acid unit via an amide bond, and a carbamate, methylcarbamate, or carbonate is made between the benzyl alcohol and a cytotoxic agent. See, e.g., Hamann et al. (2005) Expert Opin. Ther. Patents (2005) 15:1087-1103. In one embodiment, the spacer unit is p-aminobenzyloxycarbonyl (PAB). In certain embodiments, the phenylene portion of a p-amino benzyl unit is substituted with Qm, wherein Q is —Ci-Cs alkyl, -0-(Ci-Cs alkyl), -halogen, -nitro or -cyano; and m is an integer ranging from 0-4. Examples of self-immolative spacer units further include, but are not limited to, aromatic compounds that are electronically similar to p-aminobenzyl alcohol (see, e.g., US 2005/0256030 A1), such as 2-aminoimidazol-5-methanol derivatives (Hay et al. (1999) Bioorg. Med. Chem. Lett. 9:2237) and ortho- or para-aminobenzylacetals. Spacers can be used that undergo cyclization upon amide bond hydrolysis, such as substituted and unsubstituted 4-aminobutyric acid amides (Rodrigues et al., Chemistry Biology, 1995, 2, 223); appropriately substituted bicyclo[2.2.1] and bicyclo[2.2.2]ring systems (Storm, et al., /. Amer. Chem. Soc, 1972, 94, 5815); and 2-aminophenylpropionic acid amides (Amsberry, et al., /. Org. Chem., 1990, 55, 5867).

Elimination of amine-containing drugs that are substituted at the α-position of glycine (Kingsbury, et al., /. Med. Chem., 1984, 27, 1447) are also examples of self-immolative spacers useful in ADCs.

In one embodiment, a spacer unit is a branched bis(hydroxymethyl) styrene (BHMS) unit as depicted below, which can be used to incorporate and release multiple drugs.

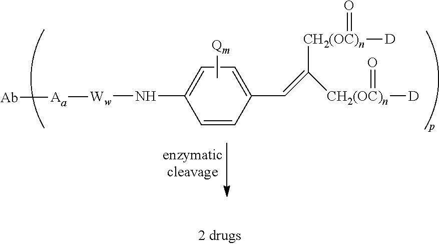

wherein Q is —Ci-Cs alkyl, -0-(Ci-Cs alkyl), -halogen, -nitro or -cyano; m is an integer ranging from 0-4; n is 0 or 1; and p ranges ranging from 1 to about 20.

In another embodiment, linker L may be a dendritic type linker for covalent attachment of more than one drug moiety through a branching, multifunctional linker moiety to an antibody (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where a cysteine engineered antibody bears only one reactive cysteine thiol group, a multitude of drug moieties may be attached through a dendritic linker.

Exemplary linker components and combinations thereof are shown below in the context of ADCs of Formula II:

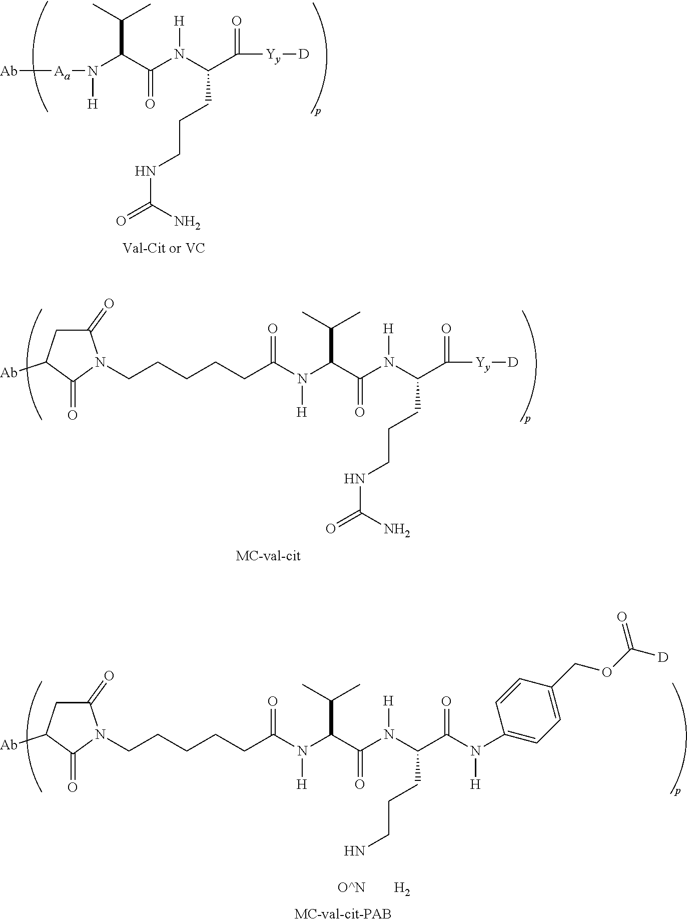

Linkers components, including stretcher, spacer, and amino acid units, may be synthesized by methods known in the art, such as those described in US 2005-0238649 A1. Additional non-limiting examples of linkers include those described in WO 2015095953, the disclosure of which is herein incorporated by reference in its entirety.

b. Exemplary Drug Moieties

### (1) Maytansine and Maytansinoids

In some embodiments, an immunoconjugate comprises an antibody conjugated to one or more maytansinoid molecules. Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody-drug conjugates because they are: (i) relatively accessible to prepare by fermentation or chemical modification or derivatization of fermentation products, (ii) amenable to derivatization with functional groups suitable for conjugation through disulfide and non-disulfide linkers to antibodies, (iii) stable in plasma, and (iv) effective against a variety of tumor cell lines.

Maytansine compounds suitable for use as maytansinoid drug moieties are well known in the art and can be isolated from natural sources according to known methods or produced using genetic engineering and fermentation techniques (U.S. Pat. No. 6,790,952; US 2005/0170475; Yu et al (2002) PNAS 99:7968-7973). Maytansinol and maytansinol analogues may also be prepared synthetically according to known methods.

Exemplary maytansinoid drug moieties include those having a modified aromatic ring, such as: C-19-dechloro (U.S. Pat. No. 4,256,746) (prepared by lithium aluminum hydride reduction of ansamytocin P2); C-20-hydroxy (or C-20-demethyl)+/−C-19-dechloro (U.S. Pat. Nos. 4,361,650 and 4,307,016) (prepared by demethylation using Streptomyces ox Actinomyces or dechlorination using LAH); and C-20-demethoxy, C-20-acyloxy (—OCOR), +/−dechloro (U.S. Pat. No. 4,294,757) (prepared by acylation using acyl chlorides) and those having modifications at other positions.

Exemplary maytansinoid drug moieties also include those having modifications such as: C-9-SH (U.S. Pat. No. 4,424,219) (prepared by the reaction of maytansinol with H₂S or P2S5); C-14-alkoxymethyl (demethoxy/CH2 OR) (U.S. Pat. No. 4,331,598); C-14-hydroxymethyl or acyloxymethyl (CH₂OH or CH₂OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudlflora); C-I 8-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Many positions on maytansine compounds are known to be useful as the linkage position, depending upon the type of link. For example, for forming an ester linkage, the C-3 position having a hydroxyl group, the C-14 position modified with hydroxymethyl, the C-15 position modified with a hydroxyl group and the C-20 position having a hydroxyl group are all suitable (U.S. Pat. No. 5,208,020; U.S. RE39151; U.S. Pat. Nos. 6,913,748; 7,368,565; US 2006/0167245; US 2007/0037972).

Maytansinoid drug moieties include those having the structure:

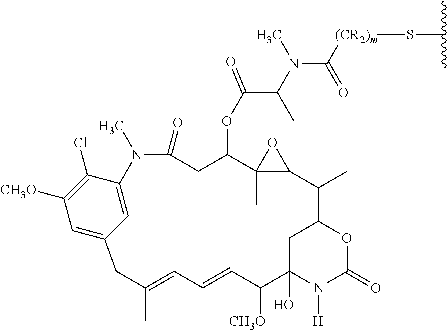

where the wavy line indicates the covalent attachment of the sulfur atom of the maytansinoid drug moiety to a linker of an ADC. R may independently be H or a C₁-C₆ alkyl. The alkylene chain attaching the amide group to the sulfur atom may be methanyl, ethanyl, or propyl, i.e., m is 1, 2, or 3 (U.S. Pat. No. 633,410; U.S. Pat. Nos. 5,208,020; 7,276,497; Chari et al (1992) Cancer Res. 52:127-131; Liu et al (1996) Proc. Natl. Acad. Sci USA 93:8618-8623).

All stereoisomers of the maytansinoid drug moiety are contemplated for the compounds of the invention, i.e. any combination of R and S configurations at the chiral carbons of D. In one embodiment, the maytansinoid drug moiety will have the following stereochemistry:

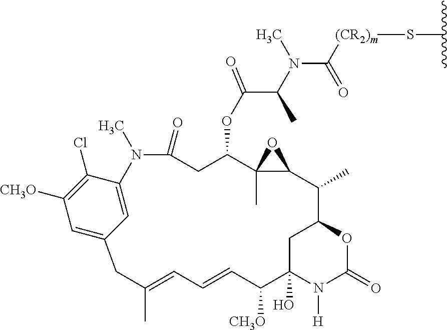

Exemplary embodiments of maytansinoid drug moeities include: DM1; DM3; and DM4, having the structures;

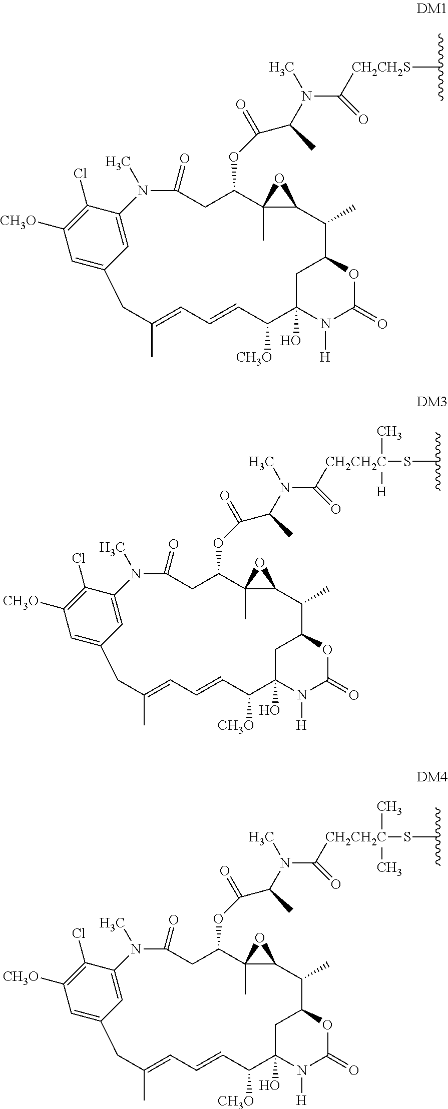

wherein the wavy line indicates the covalent attachment of the sulfur atom of the drug to a linker (L) of an antibody-drug conjugate. (WO 2005/037992; US 2005/0276812 AI).

Other exemplary maytansinoid antibody-drug conjugates have the following structures and abbreviations, (wherein Ab is antibody and p is 1 to about 8):

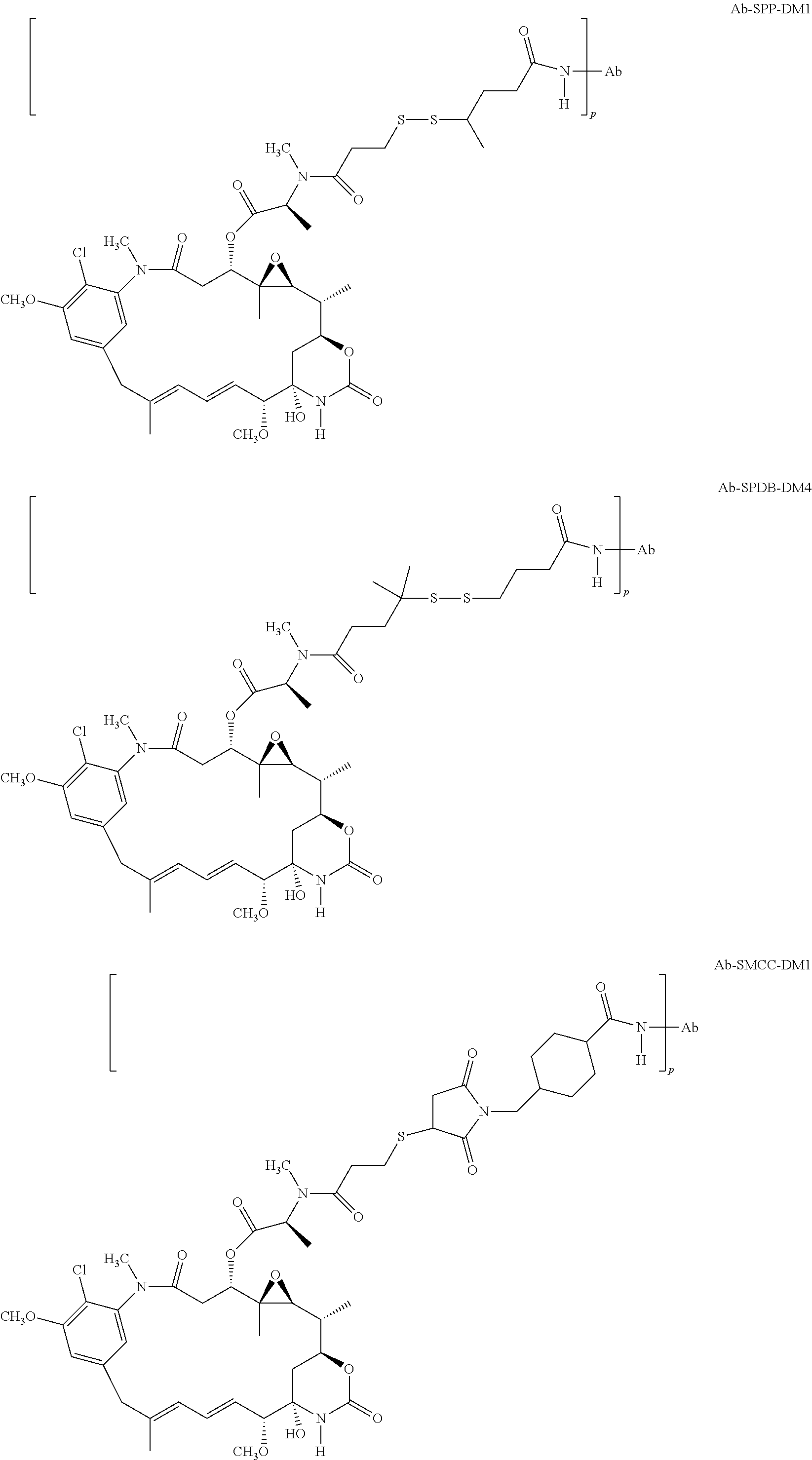

In one embodiment, the antibody-drug conjugate is formed where DM4 is linked through an SPDB linker to a thiol group of the antibody (see U.S. Pat. Nos. 6,913,748 and 7,276,497 incorporated herein by reference in their entirety).

Exemplary antibody-drug conjugates where DM1 is linked through a BMPEO linker to a thiol group of the antibody have the structure and abbreviation:

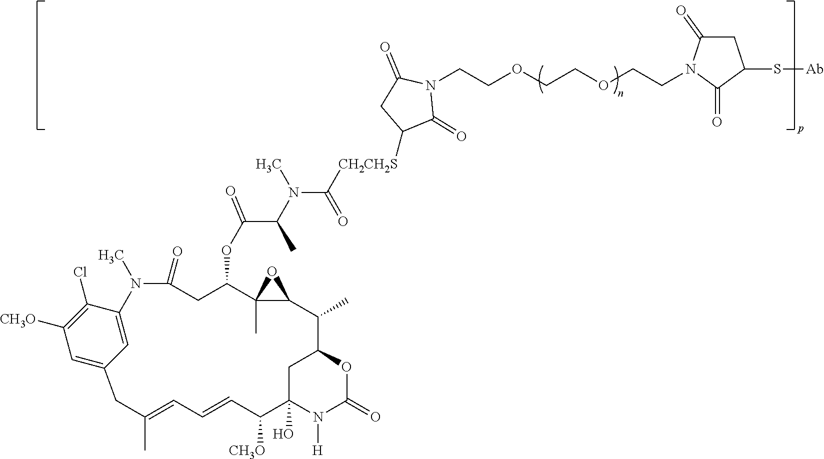

where Ab is antibody; n is 0, 1, or 2; and p is 1, 2, 3, or 4.

Immunoconjugates containing maytansinoids, methods of making the same, and their therapeutic use are disclosed, for example, in Erickson, et al (2006) Cancer Res. 66 (8): 4426-4433; U.S. Pat. Nos. 5,208,020, 5,416,064, US 2005/0276812 A1, and European Patent EP 0 425 235 BI, the disclosures of which are hereby expressly incorporated by reference.

Antibody-maytansinoid conjugates are prepared by chemically linking an antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). Maytansinoids can be synthesized by known techniques or isolated from natural sources. Suitable maytansinoids are disclosed, for example, in U.S. Pat. No. 5,208,020 and in the other patents and nonpatent publications referred to hereinabove, such as maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 BI; Chari et al. Cancer Research 52:127-131 (1992); and US 2005/016993 A1, the disclosures of which are hereby expressly incorporated by reference. Antibody-maytansinoid conjugates comprising the linker component SMCC may be prepared as disclosed in US 2005/0276812 A1, “Antibody-drug conjugates and Methods.” The linkers comprise disulfide groups, thioether groups, acid labile groups, photolabile groups, peptidase labile groups, or esterase labile groups, as disclosed in the above-identified patents. Additional linkers are described and exemplified herein.

Conjugates of the antibody and maytansinoid may be made using a variety of bifunctional protein coupling agents such as N-succinimidyl-3-(2-pyridyldithio) propionate (SPDP), succinimidyl-4-(N-maleimidomethyl) cyclohexane-I-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In certain embodiments, the coupling agent is N-succinimidyl-3-(2-pyridyldithio) propionate (SPDP) (Carlsson et al., Biochem. J. 173:723-737 (1978)) or N-succinimidyl-4-(2-pyridylthio) pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydro xymethyl, the C-15 position modified with a hydroxyl group, and the C-20 position having a hydroxyl group. In one embodiment, the linkage is formed at the C-3 position of maytansinol or a maytansinol analogue.

### (2) Auristatins and Dolastatins

In some embodiments, an immunoconjugate comprises an antibody conjugated to dolastatin or a dolastatin peptidic analog or derivative, e.g., an auristatin (U.S. Pat. Nos. 5,635,483; 5,780,588). Dolastatins and auristatins have been shown to interfere with microtubule dynamics, GTP hydrolysis, and nuclear and cellular division (Woyke et al (2001) Antimicrob. Agents and Chemother. 45 (12): 3580-3584) and have anticancer (U.S. Pat. No. 5,663,149) and antifungal activity (Pettit et al (1998) Antimicrob. Agents Chemother. 42:2961-2965). The dolastatin or auristatin drug moiety may be attached to the antibody through the N (amino) terminus or the C (carboxyl) terminus of the peptidic drug moiety (WO 02/088172). Exemplary auristatin embodiments include the N-terminus linked monomethylauristatin drug moieties DE and DF (US 2005/0238649, disclosed in Senter et al, Proceedings of the American Association for Cancer Research, Volume 45, Abstract Number 623, presented Mar. 28, 2004, the disclosure of which is expressly incorporated by reference in its entirety).

A peptidic drug moiety may be selected from Formulas DE and DF below:

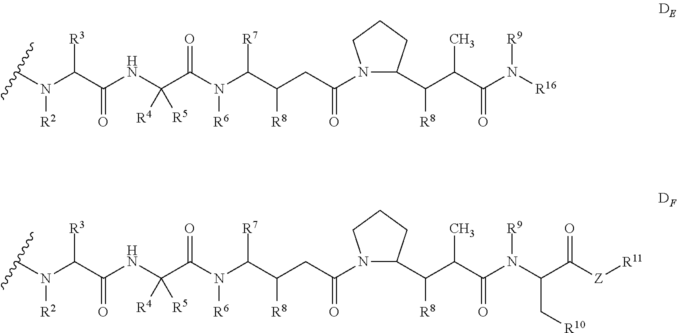

wherein the wavy line of DE and DF indicates the covalent attachment site to an antibody or antibody-linker component, and independently at each location:
R² is selected from H and Ci-Cs alkyl;
R³ is selected from H, Ci-Cg alkyl, C3-C8 carbocycle, aryl, Ci-Cg alkyl-aryl, Ci-Cg alkyl-(C3-Cs carbocycle), C3-C8 heterocycle and Ci-Cg alkyl-(C3-Cs heterocycle);
R⁴ is selected from H, Ci-Cg alkyl, C3-C8 carbocycle, aryl, Ci-Cg alkyl-aryl, Ci-Cg alkyl-(C3-Cs carbocycle), C3-C8 heterocycle and Ci-Cg alkyl-(C3-Cs heterocycle);
R⁵ is selected from H and methyl;
or R⁴ and R⁵ jointly form a carbocyclic ring and have the formula —(CR^{a}R^{b})ₙ— wherein
R^{a} and R^{b} are independently selected from H, Ci-Cg alkyl and C3-C8 carbocycle and n is selected from 2, 3, 4, 5 and 6;
R⁶ is selected from H and Ci-Cg alkyl;
R⁷ is selected from H, Ci-Cs alkyl, C3-C8 carbocycle, aryl, Ci-Cs alkyl-aryl, Ci-Cs alkyl-(C3-Cs carbocycle), C3-C8 heterocycle and Ci-Cg alkyl-(C3-Cs heterocycle);
each R⁸ is independently selected from H, OH, Ci-Cs alkyl, C3-C8 carbocycle and O—(Ci-Cs alkyl);
R⁹ is selected from H and Ci-Cs alkyl;
R¹⁰ is selected from aryl or C3-C8 heterocycle;
Z is O, S, NH, or NR¹², wherein R¹² is Ci-Cs alkyl; R¹¹ is selected from H, C1-C20 alkyl, aryl, C₃-C₈ heterocycle, —(R¹³O)ₘ—R¹⁴, or —(R¹³O)ₘ—CH(R¹⁵)₂;
m is an integer ranging from 1-1000;
R¹³ is C₂-C₈ alkyl;
R¹⁴ is H or C₁-C₈ alkyl;
each occurrence of R¹⁵ is independently H, COOH, —(CH₂)—, —N(R¹⁶)₂, —(CH₂)—, —SO₃H, or —(CH₂)ₙ—SO₃—Ci-C₈ alkyl;
each occurrence of R¹⁶ is independently H, Ci-C₈ alkyl, or —(CH₂)ₙ—COOH;
R¹⁸ is selected from —C(R⁸)₂—C(R⁸)₂-aryl, —C(R⁸)₂—C(R⁸)₂—(C₃-C₈ heterocycle), and —C(R⁸)₂—C(R⁸)₂—(C₃-C₈ carbocycle); and
n is an integer ranging from 0 to 6.

In one embodiment, R³, R⁴ and R⁷ are independently isopropyl or sec-butyl and R⁵ is —H or methyl. In an exemplary embodiment, R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is-phenyl.

In an exemplary embodiment, when Z is —O—, R¹¹ is —H, methyl or t-butyl.

In one embodiment, when Z is —NH, R¹¹ is —CH(R¹⁵)₂, wherein R¹⁵ is —(CH₂)ₙ—N(R¹⁶)₂, and R¹⁶ is —Ci-Cs alkyl or —(CH₂)ₙ—COOH.

In another embodiment, when Z is —NH, R¹¹ is —CH (R¹⁵)₂, wherein R¹⁵ is —(CH₂)ₙ—SOH.

An exemplary auristatin embodiment of formula DE is MMAE, wherein the wavy line indicates the covalent attachment to a linker (L) of an antibody-drug conjugate:

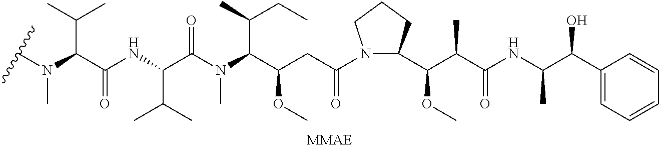 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula DF is MMAF, wherein the wavy line indicates the covalent attachment to a linker (L) of an antibody-drug conjugate (see US 2005/0238649 and Doronina et al. (2006) Bioconjugate Chem. 17:114-124):

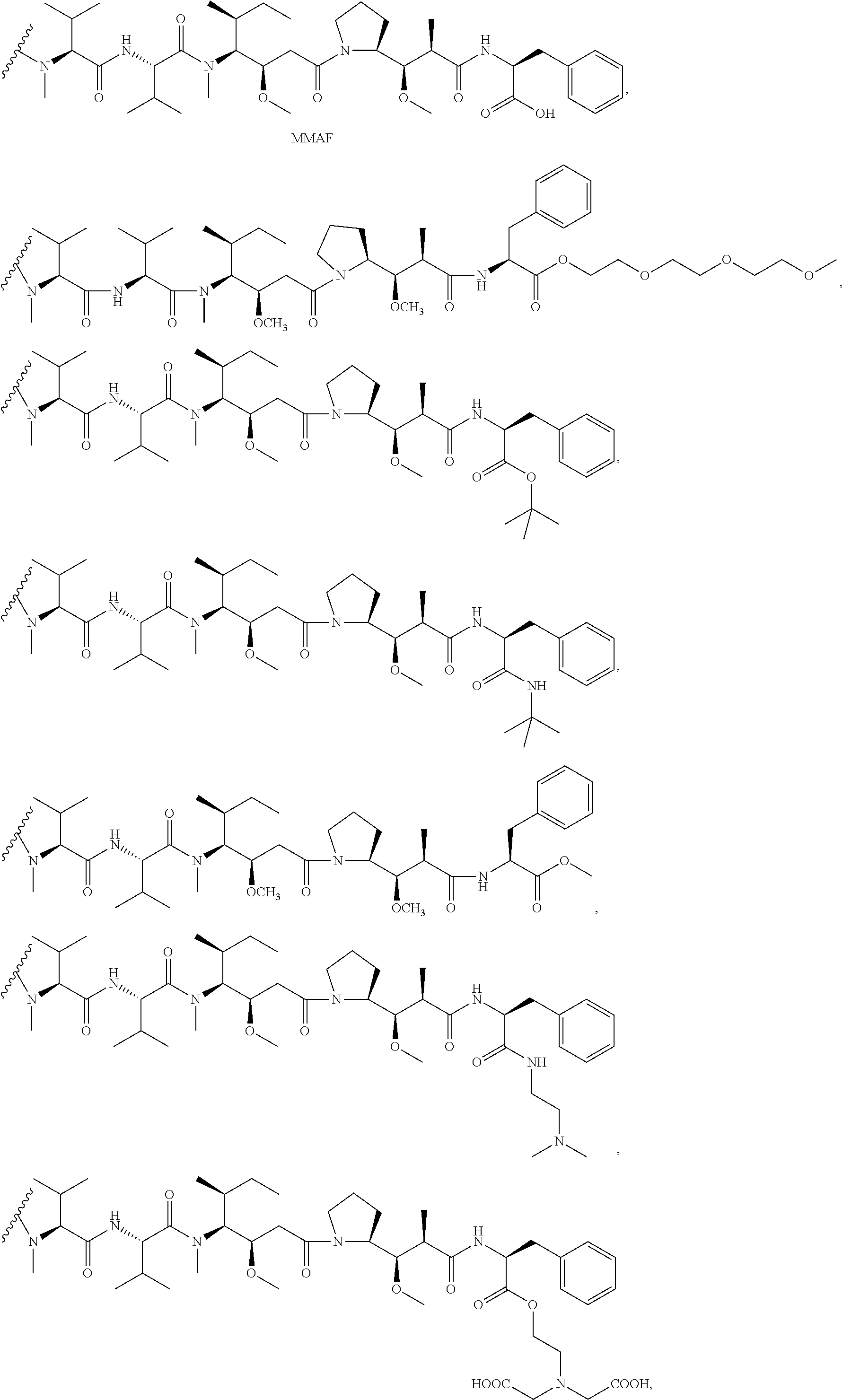

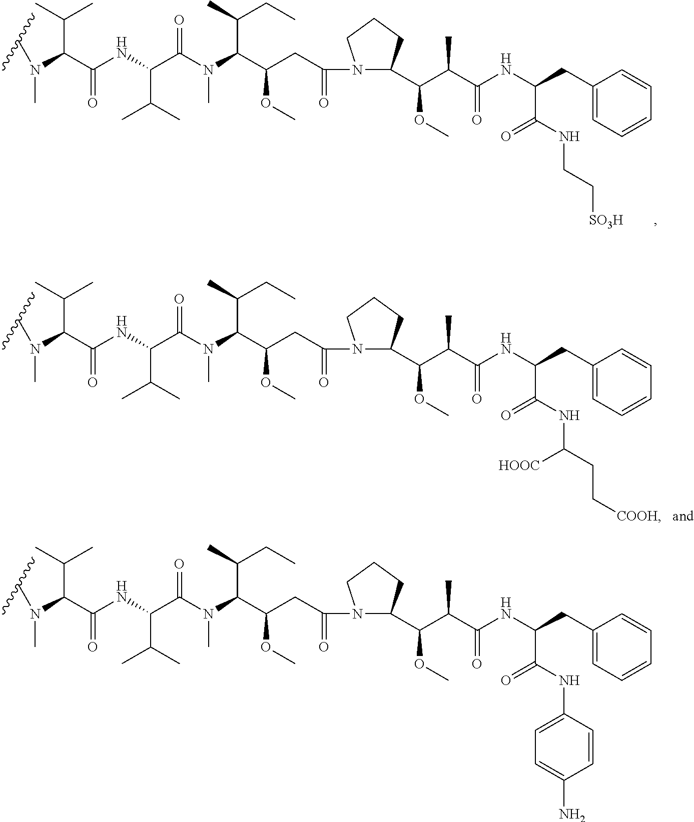

In one aspect, hydrophilic groups including but not limited to, triethylene glycol esters (TEG), as shown above, can be attached to the drug moiety at R¹¹. Without being bound by any particular theory, the hydrophilic groups assist in the internalization and non-agglomeration of the drug moiety.

Exemplary embodiments of ADCs of Formula I comprising an auristatin/dolastatin or derivative thereof are described in US 2005-0238649 and Doronina et al. (2006) Bioconjugate Chem. 17:114-124, which is expressly incorporated herein by reference.

Exemplary embodiments of ADCs of Formula I comprising MMAE or MMAF and various linker components have the following structures and abbreviations (wherein “Ab” is an antibody; p is 1 to about 8, “Val-Cit” or “vc” is a valine-citrulline dipeptide; and “S” is a sulfur atom. It will be noted that in certain of the structural descriptions of sulfur linked ADC herein the antibody is represented as “Ab-S” merely to indicate the sulfur link feature and not to indicate that a particular sulfur atom bears multiple linker-drug moieties. The left parentheses of the following structures may also be placed to the left of the sulfur atom, between Ab and S, which would be an equivalent description of the ADC of the invention described throughout herein.

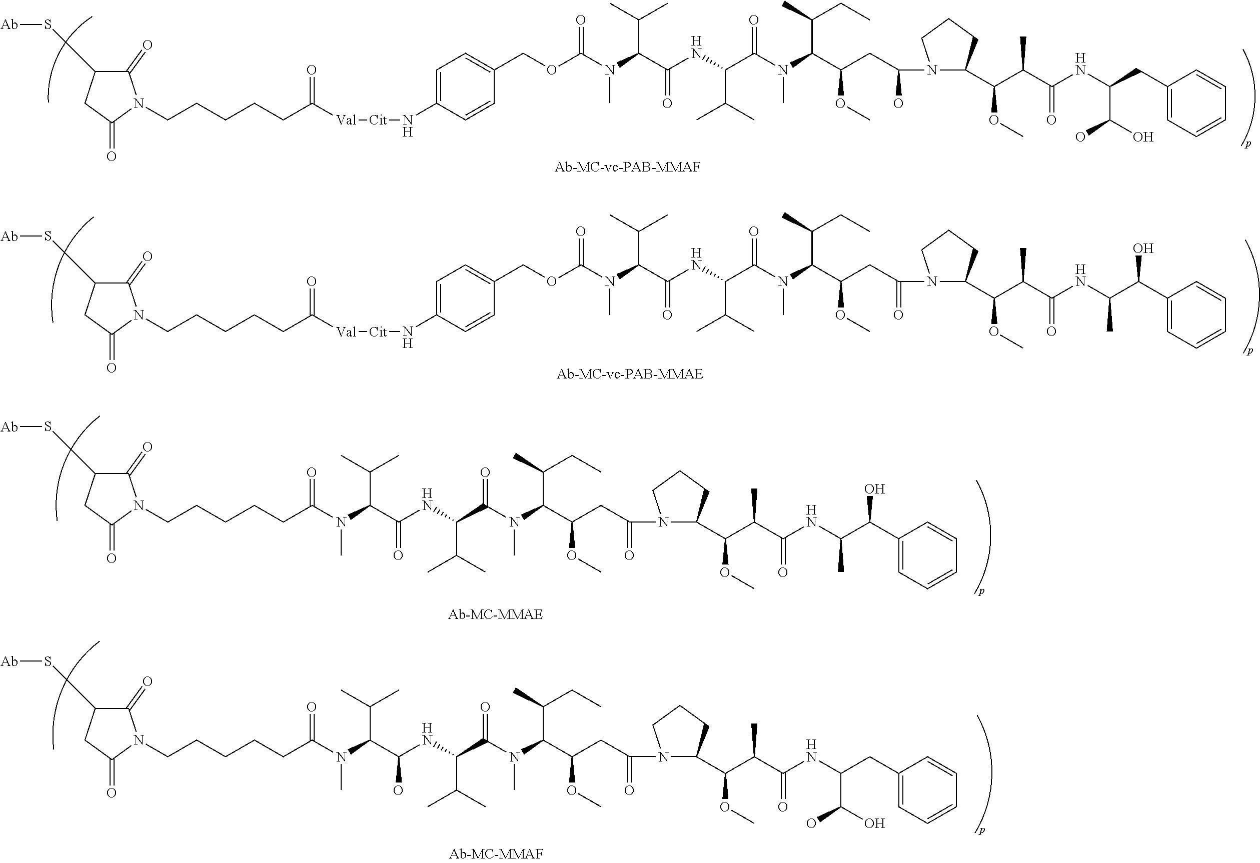

Exemplary embodiments of ADCs of Formula I comprising MMAF and various linker components further include Ab-MC-PAB-MMAF and Ab-PAB-MMAF.

Interestingly, immunoconjugates comprising MMAF attached to an antibody by a linker that is not proteolytically cleavable have been shown to possess activity comparable to immunoconjugates comprising MMAF attached to an antibody by a proteolytically cleavable linker. See, Doronina et al. (2006) Bioconjugate Chem. 17:114-124. In such instances, drug release is believed to be effected by antibody degradation in the cell. Id.

Typically, peptide-based drug moieties can be prepared by forming a peptide bond between two or more amino acids and/or peptide fragments. Such peptide bonds can be prepared, for example, according to the liquid phase synthesis method (see E. Schroder and K. Liibke, “The Peptides”, volume 1, pp 76-136, 1965, Academic Press) that is well known in the field of peptide chemistry. Auristatin/dolastatin drug moieties may be prepared according to the methods of: US 2005-0238649 AI; U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) / . Chem. Soc. Perkin Trans. 15:859-863; and Doronina (2003) Nat. Biotechnol. 21 (7): 778-784.

In particular, auristatin/dolastatin drug moieties of formula DF, such as MMAF and derivatives thereof, may be prepared using methods described in US 2005-0238649 A1 and Doronina et al. (2006) Bioconjugate Chem. 17:114-124. Auristatin/dolastatin drug moieties of formula DE, such as MMAE and derivatives thereof, may be prepared using methods described in Doronina et al. (2003) Nat. Biotech. 21:778-784. Drug-linker moieties MC-MMAF, MC-MMAE, MC-vc-PAB-MMAF, and MC-vc-PAB-MMAE may be conveniently synthesized by routine methods, e.g., as described in Doronina et al. (2003) Nat. Biotech. 21:778-784, and Patent Application Publication No. US 2005/0238649 A1, and then conjugated to an antibody of interest.

### (3) Calicheamicin

In other embodiments, the immunoconjugate comprises an antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γl¹, ai^{I}, 013¹, N-acetyl-γl¹, PSAG and Θl (Hinman et al., Cancer Research 53:3336-3342 (1993), Lode et al., Cancer Research 58:2925-2928 (1998), and the aforementioned U.S. patents to American Cyanamid). Another anti-tumor drug to which the antibody can be conjugated is QFA, which is an antifolate. Both calicheamicin and QFA have intracellular sites of action and do not readily cross the plasma membrane. Therefore, cellular uptake of these agents through antibody-mediated internalization greatly enhances their cytotoxic effects.

c. Other Cytotoxic Agents

Other antitumor agents that can be conjugated to an antibody include BCNU, streptozocin, vincristine and 5-fluorouracil, the family of agents known collectively as the LL-E33288 complex, described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO 93/21232 published Oct. 28, 1993. The present invention further contemplates an immunoconjugate formed between an antibody and a compound with nucleolytic activity (e.g., a ribonuclease or a DNA endonuclease such as a deoxyribonuclease; DNase).

In certain embodiments, an immunoconjugate may comprise a highly radioactive atom. A variety of radioactive isotopes are available for the production of radioconjugated antibodies. Examples include At²¹¹, 1¹³¹, 1¹²⁵, Y⁹⁰, Re¹⁸⁶, Re¹⁸⁸, Sm¹⁵³, Bi²¹², P³², Pb²¹² and radioactive isotopes of Lu. When the immunoconjugate is used for detection, it may comprise a radioactive atom for scintigraphic studies, for example tc^{99m} or I¹²³, or a spin label for nuclear magnetic resonance (MR) imaging (also known as magnetic resonance imaging, mri), such as iodine-123, iodine-131, indium-111, fluorine-19, carbon-13, nitrogen-15, oxygen-17, gadolinium, manganese or iron.

The radio- or other labels may be incorporated in the immunoconjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80:49-57 can be used to incorporate iodine-123. “Monoclonal Antibodies in Immunoscintigraphy” (Chatal, CRC Press 1989) describes other methods in detail.

In certain embodiments, an immunoconjugate may comprise an antibody conjugated to a prodrug-activating enzyme that converts a prodrug (e.g., a peptidyl chemotherapeutic agent, see WO 81/01145) to an active drug, such as an anti-cancer drug. Such immunoconjugates are useful in antibody-dependent enzyme-mediated prodrug therapy (“ADEPT”). Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. Enzymes may be covalently bound to antibodies by recombinant DNA techniques well known in the art. See, e.g., Neuberger et al., Nature 312:604-608 (1984).

d. Drug Loading

Drug loading is represented by p, the average number of drug moieties per antibody in a molecule of Formula I. Drug loading may range from 1 to 20 drug moieties (D) per antibody. ADCs of Formula I include collections of antibodies conjugated with a range of drug moieties, from 1 to 20. The average number of drug moieties per antibody in preparations of ADC from conjugation reactions may be characterized by conventional means such as mass spectroscopy, ELISA assay, and HPLC. The quantitative distribution of ADC in terms of p may also be determined. In some instances, separation, purification, and characterization of homogeneous ADC where p is a certain value from ADC with other drug loadings may be achieved by means such as reverse phase HPLC or electrophoresis.

Pharmaceutical formulations of Formula I antibody-drug conjugates may thus be a heterogeneous mixture of such conjugates with antibodies linked to 1, 2, 3, 4, or more drug moieties.

For some antibody-drug conjugates, p may be limited by the number of attachment sites on the antibody. For example, where the attachment is a cysteine thiol, as in the exemplary embodiments above, an antibody may have only one or several cysteine thiol groups, or may have only one or several sufficiently reactive thiol groups through which a linker may be attached. In certain embodiments, higher drug loading, e.g. p>5, may cause aggregation, insolubility, toxicity, or loss of cellular permeability of certain antibody-drug conjugates. In certain embodiments, the drug loading for an ADC of the invention ranges from 1 to about 8; from about 2 to about 6; or from about 3 to about 5. Indeed, it has been shown that for certain ADCs, the optimal ratio of drug moieties per antibody may be less than 8, and may be about 2 to about 5. See US 2005-0238649 A1.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent followed by drug moiety reagent, then the resulting product is a mixture of ADC compounds with a distribution of one or more drug moieties attached to an antibody. The average number of drugs per antibody may be calculated from the mixture by a dual ELISA antibody assay, which is specific for antibody and specific for the drug. Individual ADC molecules may be identified in the mixture by mass spectroscopy and separated by HPLC, e.g. hydrophobic interaction chromatography (see, e.g., McDonagh et al (2006) Prot. Engr. Design & Selection 19 (7): 299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous ADC with a single loading value may be isolated from the conjugation mixture by electrophoresis or chromatography.

### I. Articles of Manufacture and Kits

Another embodiment of the invention is an article of manufacture containing materials useful for the treatment, prevention and/or diagnosis of GPC3-expressing cancer. The article of manufacture comprises a container and a label or package insert on or associated with the container. Suitable containers include, for example, bottles, vials, syringes, etc. The containers may be formed from a variety of materials such as glass or plastic. The container holds a composition which is effective for treating, preventing and/or diagnosing the cancer condition and may have a sterile access port (for example the container may be an intravenous solution bag or a vial having a stopper pierceable by a hypodermic injection needle). At least one active agent in the composition is an anti-GPC3 antibody of the invention. In some embodiments, the label or package insert indicates that the composition is used for treating cancer. The label or package insert will further comprise instructions for administering the antibody composition to the cancer patient. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for GPC3-expressing cell killing assays, for purification or immunoprecipitation of GPC3 polypeptide from cells, IHC analysis of GPC3-expressing cells, and the like. For isolation and purification of GPC3 polypeptide, the kit can contain an anti-GPC3 antibody coupled to beads (e.g., sepharose beads). Kits can be provided which contain the antibodies for detection and quantitation of GPC3 polypeptide in vitro, e.g., in an ELISA or a Western blot. As with the article of manufacture, the kit comprises a container and a label or package insert on or associated with the container. The container holds a composition comprising at least one anti-GPC3 antibody of the invention. Additional containers may be included that contain, e.g., diluents and buffers, control antibodies. The label or package insert may provide a description of the composition as well as instructions for the intended in vitro or detection use.

In one embodiment, reagents for performing an IHC in vitro diagnostic (IVD) assay as herein disclosed can be provided in the form of a kit or packet. Except for the tissue preparation itself, some or all of the materials required for the assay can be provided in the kit. The kit can include but is not limited to, for example, slides for mounting tissue preparations, heat inactivating epitope retrieval (HIER) buffer, optionally a highly stable form of hydrogen peroxide for blocking endogenous peroxidase, a universal blocking reagent used for reducing nonspecific staining often found with IHC, anti-GPC3 primary antibody (e.g., 204) in diluted or undiluted form, anti-mouse secondary antibody appropriately labeled depending on the particular detection scheme, materials for visualizing the detection of antibody-antigen complex formation (e.g., DAB in the case where the secondary antibody is labeled with HRP), a stop reagent, hematoxylin for use as a nuclear counterstain, and the like.

The kit or packet may also include instructions and items for the collection or transport of a patient sample (e.g., tissue preparation) to a healthcare provider, or for receiving a sample from a healthcare provider, or for performing the evaluative methods described herein. For example, besides instructional information, a kit or packet featured in the invention can include one or more tools used in tumor biopsy.

The kit can include one or more containers for the reagents required for the IHC IVD assay. The reagents can be provided in a concentration suitable for use in the assay or with instructions for dilution for use in the assay. In some embodiments, the kit contains separate containers, dividers or compartments for the assay components, and the informational material. For example, the assay components can be contained in a bottle or vial, and the informational material can be contained in a plastic sleeve or packet. In other embodiments, the separate elements of the kit are contained within a single, undivided container. For example, an assay reagent is contained in a bottle or vial that has attached thereto the informational material in the form of a label. In some embodiments, the kit includes a plurality (e.g., a pack) of individual containers, each containing one or more unit forms (e.g., for use with one assay) of an assay component. For example, the kit includes a plurality of ampoules, foil packets, or blister packs, each containing a single unit of assay reagent for use in the IHC IVD of the present disclosure. The containers of the kits can be air tight and/or waterproof. The container can be labeled for use.

The informational material of a kit or packet is not limited in its form. In many cases, the informational material, e.g., instructions, is provided in printed matter, e.g., a printed text, drawing, and/or photograph, e.g., a label or printed sheet. However, the informational material can also be provided in other formats, such as computer readable material, video recording, or audio recording. In another embodiment, the informational material of the kit is contact information, e.g., a physical address, email address, website, or telephone number, where a user of the kit or packet can obtain substantive information about how to find the information required for the IHC IVD analysis e.g., where and how to identify prior treatments administered to a subject, and how to perform the assay to determine GPC3 expression on cells of a tissue preparation. The informational material can also be provided in any combination of formats.

In some embodiments, a tissue preparation is provided to an assay provider, e.g., a service provider (such as a third party facility) or a healthcare provider, who evaluates the sample in an assay and provides a read out. For example, in one embodiment, an assay provider receives a tissue preparation from a subject, such as a tumor biopsy sample, and evaluates the sample using the IHC IVD assay described herein, and determines that the sample contains cells that express membrane-bound GPC3. In some embodiments, the assay provider, e.g., a service provider or healthcare provider, can further determine, e.g., by contacting a healthcare provider or a database service provider, any amount of prior anti-GPC3 therapy that a patient has received or whether a patient has previously received treatment with any other relevant immunotherapy or other cancer therapy (e.g., chemotherapy).

The assay provider can further determine based on the results of the assay that the subject is either a candidate to begin or continue treatment with an anti-GPC3 therapy, such as an anti-GPC3 antibody or an anti-GPC3 CAR-T therapy, or that the subject is not a candidate to begin or continue such treatment. In a case where the patient has already been receiving such an anti-GPC3 therapy, based on the results of the assay the assay provider can potentially determine whether any adjustments to the current therapy in terms of dosing, dosing interval, and the like, should or could be made. Such determinations may be made by following instructions included within the kit. In other words, such determinations are not simply “in the head” of a person working at the assay provider, but rather may be based on instructions included in the kit and defined by the IHC IVD assay itself.

The assay provider can provide the results of the IHC IVD assay, and optionally, conclusions regarding one or more of diagnosis, prognosis, or appropriate therapy options to, for example, a healthcare provider, or patient, or an insurance company, in any suitable format, such as by mail or electronically, or through an online database. The information collected and provided by the assay provider can be stored in a database.

Thus, in one aspect, the invention provides an article of manufacture comprising a container; and a composition contained within the container, wherein the composition comprises one or more GPC3 antibodies or CAR modified immune cell, preferably a CAR-T or CAR-NK cell, of the invention. In one embodiment, the composition comprises a nucleic acid of the invention. In one embodiment, a composition comprising an antibody or CAR modified immune cell, preferably a CAR-T or CAR-NK cell, further comprises a carrier, which in some embodiments is pharmaceutically acceptable. In one embodiment, an article of manufacture of the invention further comprises instructions for administering the composition (e.g., the antibody) to a subject.

In one aspect, the invention provides a kit comprising a first container comprising a composition comprising one or more GPC3 antibodies or CAR modified immune cells, preferably a CAR-T or CAR-NK cells, of the invention; and a second container comprising a buffer. In one embodiment, the buffer is pharmaceutically acceptable. In one embodiment, a kit further comprises instructions for administering the composition (e.g., the antibody) to a subject.

In one aspect, the invention provides use of an article of manufacture of the invention in the preparation of a medicament for the therapeutic and/or prophylactic treatment of a disease, such as a cancer, a tumor and/or a cell proliferative disorder.

In one aspect, the invention provides use of a kit of the invention in the preparation of a medicament for the therapeutic and/or prophylactic treatment of a disease, such as a cancer, a tumor and/or a cell proliferative disorder.

### III. FURTHER METHODS OF USING ANTI-GPC3 ANTIBODIES

### A. Therapeutic Methods

An antibody of the invention may be used in, for example, in vitro, ex vivo, and in vivo therapeutic methods. In one aspect, the invention provides methods for inhibiting cell growth or proliferation, either in vivo or in vitro, the method comprising exposing a cell to an anti-GPC3 antibody under conditions permissive for binding of the antibody to GPC3. “Inhibiting cell growth or proliferation” means decreasing a cell's growth or proliferation by at least 10%, 20%, 30%, 40%, 50%, 60%, 70%, 80%, 90%, 95%, or 100%, and includes inducing cell death. In certain embodiments, the cell is a tumor cell. In certain embodiments, the cell is a xenograft, e.g., as exemplified herein. The antibodies may also (i) inhibit the growth or proliferation of a cell to which they bind; (ii) induce the death of a cell to which they bind; (iii) inhibit the delamination of a cell to which they bind; (iv) inhibit the metastasis of a cell to which they bind; or (v) inhibit the vascularization of a tumor comprising a cell to which they bind.

In one aspect, an antibody of the invention is used to treat or prevent a cell proliferative disorder. In certain embodiments, the cell proliferative disorder is associated with increased expression and/or activity of GPC3. For example, in certain embodiments, the cell proliferative disorder is associated with increased expression of GPC3 on the surface of a cell. In certain embodiments, the cell proliferative disorder is a tumor or a cancer.

In one aspect, the invention provides methods for treating a cell proliferative disorder comprising administering to an individual an effective amount of an anti-GPC3 antibody. In one embodiment, an anti-GPC3 antibody can be used in a method for binding GPC3 in an individual suffering from a disorder associated with increased GPC3 expression and/or activity, the method comprising administering to the individual the antibody such that GPC3 in the individual is bound. In one embodiment, the GPC3 is human GPC3, and the individual is a human individual. An anti-GPC3 antibody can be administered to a human for therapeutic purposes. Moreover, an anti-GPC3 antibody can be administered to a non-human mammal expressing GPC3 with which the antibody cross-reacts (e.g., a primate, pig, rat, or mouse) for veterinary purposes or as an animal model of human disease. Regarding the latter, such animal models may be useful for evaluating the therapeutic efficacy of antibodies of the invention (e.g., testing of dosages and time courses of administration).

An antibody of the invention (and any additional therapeutic agent or adjuvant) can be administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Dosing can be by any suitable route, e.g. by injections, such as intravenous or subcutaneous injections, depending in part on whether the administration is brief or chronic.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### B. Activity Assays

Anti-GPC3 antibodies of the invention may be characterized for their physical/chemical properties and/or biological activities by various assays known in the art.

### 1. Activity Assays

In one aspect, assays are provided for identifying anti-GPC3 antibodies thereof having biological activity. Biological activity may include, e.g., the ability to inhibit cell growth or proliferation (e.g., “cell killing” activity), or the ability to induce cell death, including programmed cell death (apoptosis). Antibodies having such biological activity in vivo and/or in vitro are also provided.

In certain embodiments, an anti-GPC3 antibody is tested for its ability to inhibit cell growth or proliferation in vitro. Assays for inhibition of cell growth or proliferation are well known in the art. Certain assays for cell proliferation, exemplified by the “cell killing” assays described herein, measure cell viability. One such assay is the CellTiter-Glo™ Luminescent Cell Viability Assay, which is commercially available from Promega (Madison, WI). That assay determines the number of viable cells in culture based on quantitation of ATP present, which is an indication of metabolically active cells. See Crouch et al (1993) J. Immunol. Meth. 160:81-88, U.S. Pat. No. 6,602,677. The assay may be conducted in 96- or 384-well format, making it amenable to automated high-throughput screening (HTS). See Cree et al (1995) Anticancer Drugs 6:398-404. The assay procedure involves adding a single reagent (CellTiter-Glo® Reagent) directly to cultured cells. This results in cell lysis and generation of a luminescent signal produced by a luciferase reaction. The luminescent signal is proportional to the amount of ATP present, which is directly proportional to the number of viable cells present in culture. Data can be recorded by luminometer or CCD camera imaging device. The luminescence output is expressed as relative light units (RLU).

Another assay for cell proliferation is the “MTT” assay, a colorimetric assay that measures the oxidation of 3-(4,5-dimethylthiazol-2-yl)-2,5-diphenyltetrazolium bromide to formazan by mitochondrial reductase. Like the CellTiter-Glo™ assay, this assay indicates the number of metabolically active cells present in a cell culture. See, e.g., Mosmann (1983) J. Immunol. Meth. 65:55-63, and Zhang et al. (2005) Cancer Res. 65:3877-3882.

In one aspect, an anti-GPC3 antibody is tested for its ability to induce cell death in vitro. Assays for induction of cell death are well known in the art. In some embodiments, such assays measure, e.g., loss of membrane integrity as indicated by uptake of propidium iodide (PI), trypan blue (see Moore et al. (1995) Cytotechnology, 17:1-11), or 7AAD. In an exemplary PI uptake assay, cells are cultured in Dulbecco's Modified Eagle Medium (D-MEM): Ham's F-12 (50:50) supplemented with 10% heat-inactivated FBS (Hyclone) and 2 mM L-glutamine. Thus, the assay is performed in the absence of complement and immune effector cells. Cells are seeded at a density of 3×106 per dish in 100×20 mm dishes and allowed to attach overnight. The medium is removed and replaced with fresh medium alone or medium containing various concentrations of the antibody. The cells are incubated for a 3-day time period. Following treatment, monolayers are washed with PBS and detached by trypsinization. Cells are then centrifuged at 1200 rpm for 5 minutes at 4° C., the pellet resuspended in 3 ml cold Ca2+ binding buffer (10 mM Hepes, pH 7.4, 140 mM NaCl, 2.5 mM CaCl2) and aliquoted into 35 mm strainer-capped 12×75 mm tubes (1 ml per tube, 3 tubes per treatment group) for removal of cell clumps. Tubes then receive PI (10 μg/ml). Samples are analyzed using a FACSCAN™ flow cytometer and FACSCONVERT™ CellQuest software (Becton Dickinson). Antibodies which induce statistically significant levels of cell death as determined by PI uptake are thus identified.

In one aspect, an anti-GPC3 antibody is tested for its ability to induce apoptosis (programmed cell death) in vitro. An exemplary assay for antibodies that induce apoptosis is an annexin binding assay. In an exemplary annexin binding assay, cells are cultured and seeded in dishes as discussed in the preceding paragraph. The medium is removed and replaced with fresh medium alone or medium containing 0.001 to 10 μg/ml of the antibody. Following a three-day incubation period, monolayers are washed with PBS and detached by trypsinization. Cells are then centrifuged, resuspended in Ca2+ binding buffer, and aliquoted into tubes as discussed in the preceding paragraph. Tubes then receive labeled annexin (e.g. annexin V-FITC) (1 μg/ml). Samples are analyzed using a FACSCAN™ flow cytometer and FACSCONVERT™ CellQuest software (BD Biosciences). Antibodies that induce statistically significant levels of annexin binding relative to control are thus identified. Another exemplary assay for antibodies that induce apoptosis is a histone DNA ELISA colorimetric assay for detecting internucleosomal degradation of genomic DNA. Such an assay can be performed using, e.g., the Cell Death Detection ELISA kit (Roche, Palo Alto, CA).

Cells for use in any of the above in vitro assays include cells or cell lines that naturally express GPC3 or that have been engineered to express GPC3. Such cells include tumor cells that overexpress GPC3 relative to normal cells of the same tissue origin. Such cells also include cell lines (including tumor cell lines) that express GPC3 and cell lines that do not normally express GPC3 but have been transfected with nucleic acid encoding GPC3.

In one aspect, an anti-GPC3 antibody thereof is tested for its ability to inhibit cell growth or proliferation in vivo. In certain embodiments, an anti-GPC3 antibody thereof is tested for its ability to inhibit tumor growth in vivo. In vivo model systems, such as xenograft models, can be used for such testing. In an exemplary xenograft system, human tumor cells are introduced into a suitably immunocompromised non-human animal, e.g., a SCID mouse. An antibody of the invention is administered to the animal. The ability of the antibody to inhibit or decrease tumor growth is measured. In certain embodiments of the above xenograft system, the human tumor cells are tumor cells from a human patient. In certain embodiments, the human tumor cells are introduced into a suitably immunocompromised non-human animal by subcutaneous injection or by transplantation into a suitable site, such as a mammary fat pad.

### 2. Binding Assays and Other Assays

In one aspect, an anti-GPC3 antibody is tested for its antigen binding activity. For example, in certain embodiments, an anti-GPC3 antibody is tested for its ability to bind to GPC3 expressed on the surface of a cell. A FACS assay may be used for such testing.

In one aspect, competition assays may be used to identify a monoclonal antibody that competes with a monoclonal antibody comprising the variable domains of SEQ ID NO: 2 and SEQ ID NO: 4 or a chimeric antibody comprising the variable domain of the monoclonal antibody comprising the sequences of SEQ ID NO: 2 and SEQ ID NO: 4 and constant domains from IgGI for binding to GPC3. In certain embodiments, such a competing antibody binds to the same epitope (e.g., a linear or a conformational epitope) that is bound by a monoclonal antibody comprising the variable domains of SEQ ID NO: 2 and SEQ ID NO: 4 or a chimeric antibody comprising the variable domain of the monoclonal antibody comprising the sequences of SEQ ID NO: 2 and SEQ ID NO: 4 and constant domains from IgGI. Exemplary competition assays include, but are not limited to, routine assays such as those provided in Harlow and Lane (1988) Antibodies: A Laboratory Manual ch. 14 (Cold Spring Harbor Laboratory, Cold Spring Harbor, NY). Detailed exemplary methods for mapping an epitope to which an antibody binds are provided in Morris (1996) “Epitope Mapping Protocols,” in Methods in Molecular Biology vol. 66 (Humana Press, Totowa, NJ). Two antibodies are said to bind to the same epitope if each blocks binding of the other by 50% or more.

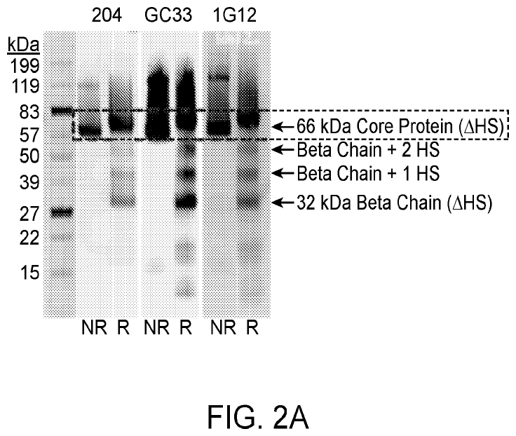

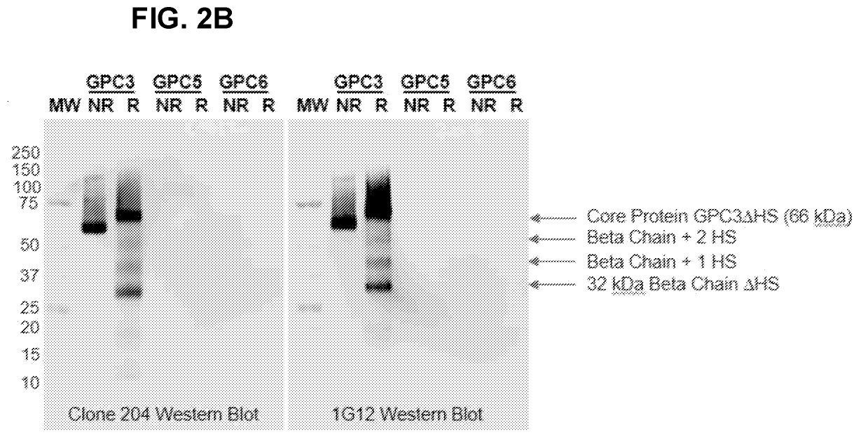

In an exemplary competition assay, immobilized GPC3 is incubated in a solution comprising a first labeled antibody that binds to GPC3 (e.g., a monoclonal antibody comprising the variable domains of SEQ ID NO: 2 and SEQ ID NO: 4 or a chimeric antibody comprising the variable domain of the monoclonal antibody comprising the sequences of SEQ ID NO: 2 and SEQ ID NO: 4 (FIG. 2) and constant domains from IgGI) and a second unlabeled antibody that is being tested for its ability to compete with the first antibody for binding to GPC3. The second antibody may be present in a hybridoma supernatant. As a control, immobilized GPC3 is incubated in a solution comprising the first labeled antibody but not the second unlabeled antibody. After incubation under conditions permissive for binding of the first antibody to GPC3, excess unbound antibody is removed, and the amount of label associated with immobilized GPC3 is measured. If the amount of label associated with immobilized GPC3 is substantially reduced in the test sample relative to the control sample, then that indicates that the second antibody is competing with the first antibody for binding to GPC3. In certain embodiments, immobilized GPC3 is present on the surface of a cell or in a membrane preparation obtained from a cell expressing GPC3 on its surface.

In one aspect, purified anti-GPC3 antibodies can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

The following examples are offered for illustrative purposes only, and are not intended to limit the scope of the present invention in any way.

All patent, patent application, and literature references cited in the present specification are hereby incorporated by reference in their entirety.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Anti-GPC3 Monoclonal Antibody Generation

### Immunization

Six-week old Balb/c mice were immunized with purified rhGPC3 protein and treated with adjuvant doublet therapy. For subsequent boosts, each mouse received rhGPC3 with adjuvant therapy every 3-4 days over the course of six weeks.

### Selection of Mouse Donors

Supplemental bleeds were obtained after the 5th and 12th injections of rhGPC3, and serum samples were collected for titer determination. To monitor the immune response, sera from immunized mice were screened by flow cytometry for anti-hGPC3 antibodies binding to hGPC3-expressing cell lines (e.g., HepG2 and RAT2 GPC3). Mice with the highest serum titers were chosen for fusions. Mice chosen for fusions were sacrificed four days after the last boost.

### Generation of Hybridomas Producing Mouse Antibodies

Lymphocytes isolated from the mouse lymph nodes were fused with non-secreting mouse myeloma cell line Sp2/0-Ag14, and these hybridoma cells were single-cell sorted into 384-well plates after a 6 day incubation period.

After a 9-day incubation period, hybridoma supernatants were screened undiluted for antigen specificity via flow cytometry. Positive clones were expanded in 96 well plates, followed by secondary screening and expansion in 24 well plates. Antibody concentration from hybridoma supernatants was quantified, isotyped/sequenced, and submitted for small scale purification.

### Sequencing Procedure

Total RNA was isolated from hybridomas. 2-step reverse transcriptase polymerase chain reaction (RT-PCR) using SMARTer® rapid amplification of cDNA ends (RACE) 5′/3′ was performed (Takara Bio, Inc., Kusatsu, Shiga, Japan). Briefly, cDNA was synthesized via reverse transcription. RACE PCR of heavy and light chains was performed using gene-specific primers for specific isotypes (Bradbury, A. 2010. Cloning Hybridoma cDNA by RACE. In: Kontermann R., Dübel S. (eds) Antibody Engineering. Springer Protocols Handbooks. Springer, Berlin, Heidelberg. https://doi.org/10.1007/978-3-642-01144-32).

RACE PCR products were subcloned using CloneJETPCR cloning kit (ThermoFisher, Waltham, MA), followed by Sanger sequencing and annotation by Kabat numbering and analysis. Specifically, the extent of the framework region and CDRs has been defined according to Kabat et al. (see, Kabat et al., Sequences of Proteins of Immunological Interest, U.S. Department of Health and Human Services, 1991). The Kabat database is maintained online (world wide web at ncbi.nlm.nih.gov/igblast/).

Sequence summary of V_{H} and V_{L} Domains

Table 1 below depicts amino acid sequences of framework regions (FRs 1-4) and complementary determining regions (CDRs 1-3) of the 204 mAb. Depicted at Table 2 are nucleic acid sequences and amino acid sequences corresponding to the heavy chain variable region and the light chain variable region of 204. Tables 3 and 4 illustrate the SEQ ID NOs corresponding to the sequences shown in Tables 1 and 2, respectively.

TABLE 1
Framework regions and complementary determining regions of 204

| GPC3 | Iso- | | | | | | | |
|---|---|---|---|---|---|---|---|---|
| clone | type | FR1 | CDR1 | FR2 | CDR2 | FR3 | CDR3 | FR4 |
| 204 | igG2b | EVQLQQSGPE | EYAMH | WVKQSH | GINPNNGVT | KATLTVDKSSSTAYMEL | GLLW | WGQGT |
| | | LVKPGASVKI | (SEQ ID | GKSLEWIG | TYNQRFKG | RSLTSEDSAVYYCAR | YAY | LVTVSA |
| | | SCKTSGYTFT | NO: 6) | (SEQ ID | (SEQ ID | (SEQ ID NO: 9) | (SEQ ID | (SEQ ID |
| | | (SEQ ID | | NO: 7) | NO: 8) | | NO: 10) | NO: 11) |
| | | NO: 5) | | | | | | |
| | Kappa | DIKMTQSPSS | KASQDIN | WFQQKP | RANRLVD | GVPSRFSGSGSGQDYS | LQYDE | FGAG |
| | | MYASLGERVT | SYLS | GKSPKTL | (SEQ ID | LTISSLEYEDMGIYYC | FPLT | TKLELK |
| | | ITC | (SEQ ID | IY | NO: 15) | (SEQ ID NO: 16) | (SEQ ID | (SEQ ID |
| | | (SEQ ID | NO: 13) | (SEQ ID | | | NO: 17) | NO: 18) |
| | | NO: 12) | | NO: 14) | | | | |

TABLE 2
VH and VL regions of 204

| GPC3 | Iso- | | |
|---|---|---|---|
| clone | type | Amino Acid Sequence | Nucleotide sequence |
| 204 | igG2b | EVQLQQSGPELVKPGASVKISCKTSGYTFTE | GAGGTCCAGCTGCAACAGTCTGGACCTGAGCTGG |
| | | YAMHWVKQSHGKSLEWIGGINPNNGVTT | TGAAGCCTGGGGCTTCAGTGAAGATATCCTGCAA |
| | | YNQRFKGKATLTVDKSSSTAYMELRSLTSED | GACTTCTGGATACACATTCACTGAATACGCCATGC |
| | | SAVYYCARGLLWYAYWGQGTLVTVSA | ACTGGGTGAAGCAGAGCCATGGAAAGAGCCTTGA |
| | | (SEQ ID NO: 2) | GTGGATTGGAGGTATTAATCCTAACAATGGTGTTA |
| | | | CTACTTACAACCAGAGGTTCAAGGGCAAGGCCACA |
| | | | TTGACTGTAGACAAGTCCTCCAGCACAGCCTACATG |
| | | | GAGCTCCGCAGCCTGACATCTGAGGATTCTGCAGTC |
| | | | TATTACTGTGCAAGAGGCCTACTATGGTATGCTTAC |
| | | | TGGGGCCAAGGGACTCTGGTCACTGTCTCTGCA |
| | | | (SEQ ID NO: 1) |
| | Kappa | DIKMTQSPSSMYASLGERVTITCKASQDINS | GACATCAAGATGACCCAGTCTCCATCTTCCATGTATGC |
| | | YLSWFQQKPGKSPKTLIYRANRLVDGVPSRF | ATCTCTAGGAGAGAGAGTCACTATCACTTGCAAGGCG |
| | | SGSGSGQDYSLTISSLEYEDMGIYYCLQYDEF | AGTCAGGACATTAATAGCTATTTAAGCTGGTTCCAGCA |
| | | PLTFGAGTKLELK | GAAACCAGGGAAATCTCCTAAGACCCTGATCTATCGTG |
| | | (SEQ ID NO: 4) | CAAACAGATTGGTAGATGGGGTCCCATCAAGGTTCAGT |
| | | | GGCAGTGGATCTGGGCAAGATTATTCTCTCACCATCAGC |
| | | | AGCCTGGAGTATGAAGATATGGGAATTTATTATTGTCTA |
| | | | CAGTATGATGAGTTTCCTCTCACGTTCGGTGCTGGGACCA |
| | | | AGCTGGAGCTGAAA |
| | | | (SEQ ID NO: 3) |

Tables 3 and 4 illustrate the SEQ ID NOs corresponding to the sequences shown in Tables 1 and 2, respectively.

TABLE 3
204 V_{H} and V_{L} SEQ ID NOs

| | Nucleotide | AA |
|---|---|---|
| V_{H} | SEQ ID NO: 1 | SEQ ID NO: 2 |
| V_{L} | SEQ ID NO: 3 | SEQ ID NO: 4 |

TABLE 4
204 FR and CDR SEQ ID NOs

| | |
|---|---|
| HFR1 | SEQ ID NO: 5 |
| HCDR1 | SEQ ID NO: 6 |
| HFR2 | SEQ ID NO: 7 |
| HCDR2 | SEQ ID NO: 8 |
| HFR3 | SEQ ID NO: 9 |
| HCDR3 | SEQ ID NO: 10 |
| HFR4 | SEQ ID NO: 11 |
| LFR1 | SEQ ID NO: 12 |
| LCDR1 | SEQ ID NO: 13 |
| LFR2 | SEQ ID NO: 14 |
| LCDR2 | SEQ ID NO: 15 |
| LFR3 | SEQ ID NO: 16 |
| LCDR3 | SEQ ID NO: 17 |
| LFR4 | SEQ ID NO: 18 |

### Example 2. Characterization of GPC.204 Monoclonal Antibody

### Cell-Based Assay Bio-Layer Inferometry (BLI) Competitive Binding Assay

A cross-competition assay was performed In-Tandem by immobilizing antigen at low levels (to minimize crowding artifacts), saturating the antigen with the anti-GPC3 antibody, and then exposing the biosensors to the competing antibodies. Streptavidin (SA) biosensors (Fortébio, Fremont, CA) were loaded with biotinylated antigen at a concentration of 1 μg/mL over 200 seconds (to final wavelength shift of 0.8 nm). The antigen-loaded biosensors were exposed to 150 nM of saturating antibody for 600 seconds (until a sustained plateau was achieved), and then to 150 nM competing antibody for 300 seconds. Antigen-loaded biosensors were also exposed to the competing antibodies after exposure to buffer only (i.e., no saturating antibody) to illustrate maximal binding to the antigen for each antibody. The binding data were analyzed using the Octet Data Analysis Software v11.0 (Sartorious, AG, Göttingen, Germany) and the “Process Epitope Binning Data” feature of the software. The resulting binding signal (nm) of each competing antibody was expressed as % binding by dividing by the maximal binding signal for each antibody (no saturating antibody).

The results of the assay are quantified in Table 5 below. In Table 5, competitive antibody binding of less than 50% was considered blocking. Binding percentage on the diagonal measures self-blocking and is expected to be close to 0%.

TABLE 5
Results of BLI competitive binding assay

| | |
|---|---|
| Saturating | % Binding of Competing Antibody |

| Antibody | 204 | GC33 |
|---|---|---|
| 204 | 18.2 | 97.2 |
| GC33 | 89.9 | 0 |
| Buffer | 100 | 100 |

The data in Table 5 indicate that 204 binds to a different GPC3 epitope as compared with GC33, because GPC3 bound by 204 did not block the binding of GP33, and GPC3 bound by GP33 did not substantially block the binding of 204.

### BLI Binding Assay

To asses binding parameters of purified mouse anti-human GPC3 monoclonal antibodies of the present disclosure, BLI binding assays were performed. Briefly, anti-mouse IgG-Fc capture (AMC) biosensors (Fortébio, Fremont, CA) were loaded with purified antibody (mouse IgG) at a concentration of 1-3 μg/mL over 300 seconds (to final wavelength shift of 0.8-1.0 nm). Humanized GC33 antibody was immobilized to anti-human IgG-Fc capture (AHC) biosensors using the same parameters. The antibody loaded biosensors were exposed to 5 concentrations of antigen (analyte) beginning at 100 nM and serially diluted 1:3. An antibody loaded biosensor was also exposed to a reference will containing kinetic buffer only (to correct for drift). Biosensors not loaded with antibody were also exposed to the antigen to check for non-specific binding to the sensors and no binding was observed (data not shown). The data from at least 4 concentrations were fit using a global 1:1 curve fit model, where the possible kinetic association and dissociation rates and the K_{D} values were determined. For the calculated constants to be accepted, binding statistics had to fall within acceptable parameters (i.e., Chi²≤3.0 and R²≥0.95).

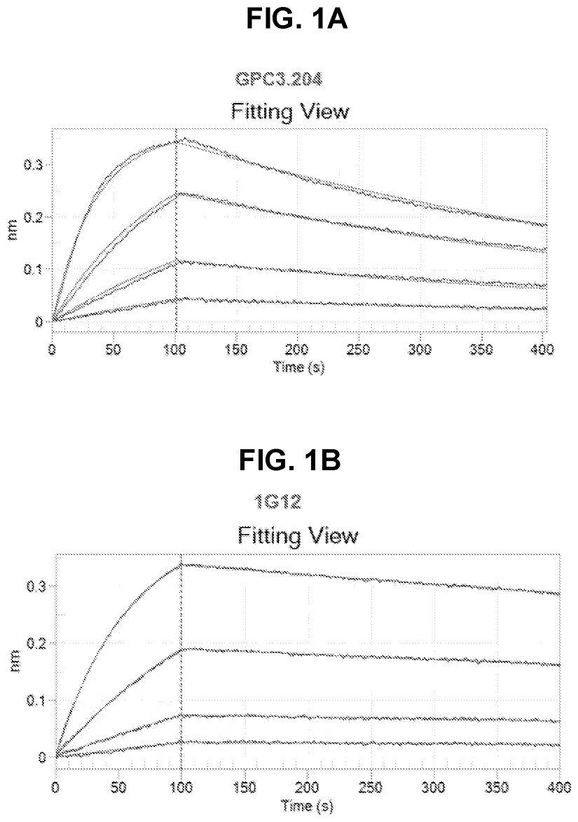

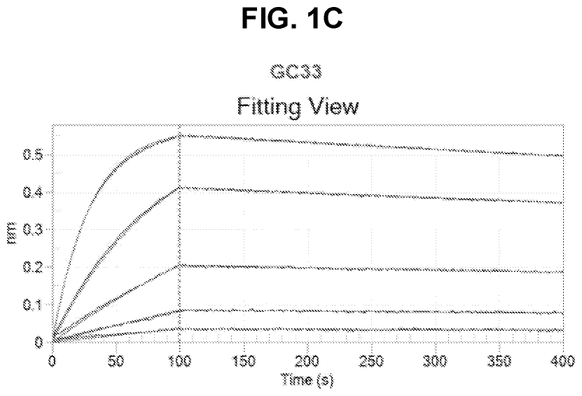

Raw data for the binding assays corresponding to the various anti-GPC3 monoclonal antibodies of the present disclosure are depicted at FIGS. 1A-1C. The determined kinetic association/dissociation rates and K_{D} values are shown in Table 6 below.

TABLE 6
Binding and kinetic parameters for 204, 1G12, and GC33

| | Epitope | K_{D} | kₐ | k_{d} | | |
|---|---|---|---|---|---|---|
| Antibody | Domain | (nM) | (1 · Ms) | (1/s) | Chi² | R² |
| 204 | C-Term | 2.5 | 8.2 × 10⁵ | 2.1 × 10⁻³ | 0.04 | 1.00 |
| 1G12 | C-Term | 3.0 | 1.7 × 10⁵ | 5.3 × 10⁻⁴ | 0.00 | 1.00 |
| GC33 | C-Term | 1.0 | 3.4 × 10⁵ | 3.4 × 10⁻⁴ | 0.02 | 1.00 |

The data depicted at Table 6 illustrates that the C-terminal 204 antibody demonstrates comparable affinity for recombinant GPC3 when compared to the other control C-terminal GPC3 antibodies 1G12 and GC33.

### Assessment of GPC3 in Cell Lysates Via Western Blot

Various tumor cell lines were grown to confluency, serum-free supernatants were collected at 24 hours and concentrated 20-fold. 4×LDS buffer (Thermofisher, Waltham, MA)±8% β-Me was added to concentrated serum-free supernatants or to rhGPC3 to a final concentration of 1×LDS±2% β-Me, and the samples were heated at 95° C. for 10 minutes. Samples were loaded on a 10-well Bis-Tris gel with 40 μL supernatant or 300 ng rhGPC3 and run at 200V for 40 minutes. Protein was transferred to nitrocellulose as per iBlot2 instructions (Thermofisher, Waltham, MA). The blot was stained with primary antibodies at 1 μg/mL in 5% BSA, 1×TBS, 0.1% Tween 20 staining diluent for 2 hours at room temperature. The blot was then stained with IRdye800 conjugated secondary antibodies (Li-Cor Biosciences, Lincoln, NE) as per Li-Cor recommendations and imaged using the Li-Cor Odyssey instrument.

FIG. 2A depicts western blotting results of antibodies of the present disclosure that recognize the C-terminus of rhGPC3. As shown, each of 204, GC33, and 1G12 detect the 32 kDa beta chain of GPC3 under reducing conditions (R) but not under non-reducing conditions (NR). FIG. 2B depicts western blotting results of the 204 and 1G12 antibodies used to probe rhGPC3, rhGPC5, and rhGPC6 under reducing and non-reducing conditions. Similar to FIG. 2A, the 32 kDa beta chain of GPC3 is detected under reducing conditions but not under non-reducing conditions. Neither the 204 antibody nor the 1G12 antibody detect rhGPC5 or rhGPC6 regardless of whether the samples were reduced or not.

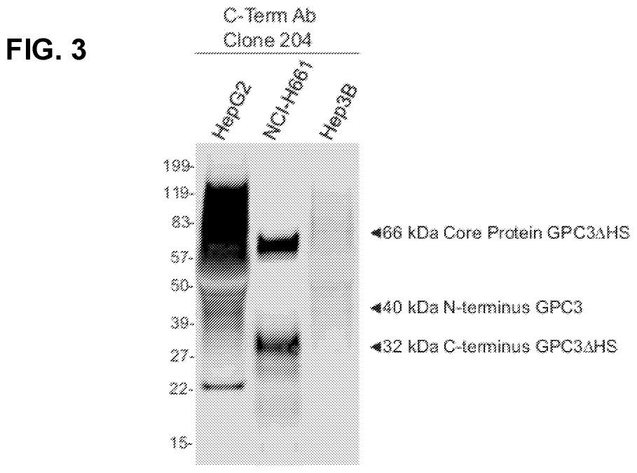

Turning to FIG. 3 depicted is a western blot analysis of soluble native human GPC3 probed with 204. Tumor cell lines tested included HepG2, NCI-H661, and Hep3B. The 32 kDa beta chain (without the HS side chains) was only detected under reducing conditions in the NCI-H661 tumor cell line supernatant. In fact, the soluble GPC3 appears to exist only as the un-glycosylated and un-sulfinated core protein in the NCI-H661 supernatant. For HepG2 and Hep3B, the smear between 37-50 kDa under reducing conditions likely represents the glycosylated, sulfinated forms of the C-terminal fragment.

ADAM Cleavage of rhGPC3

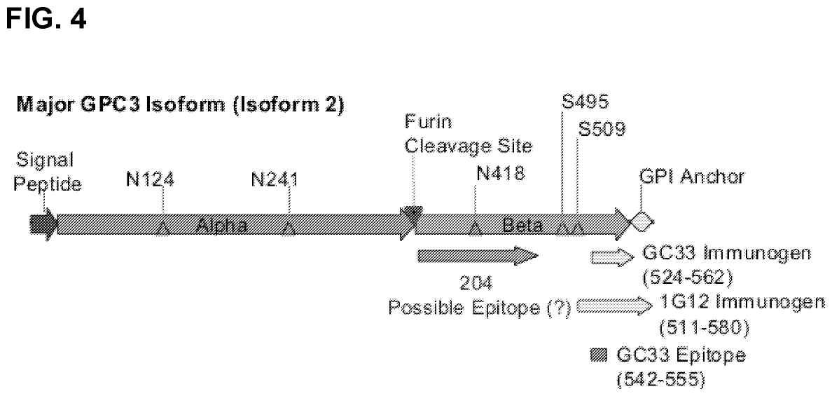

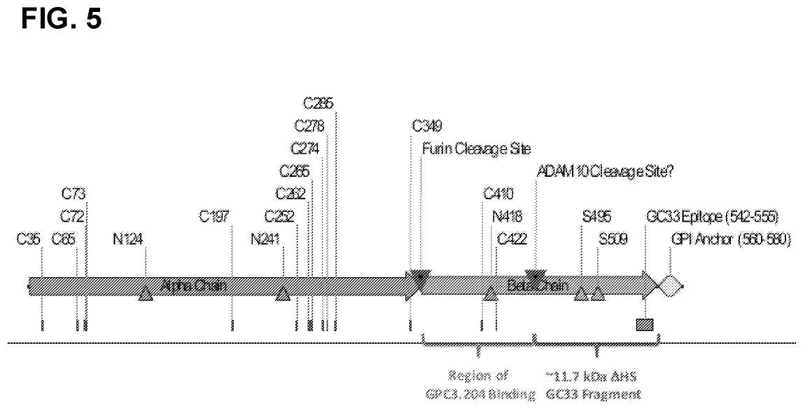

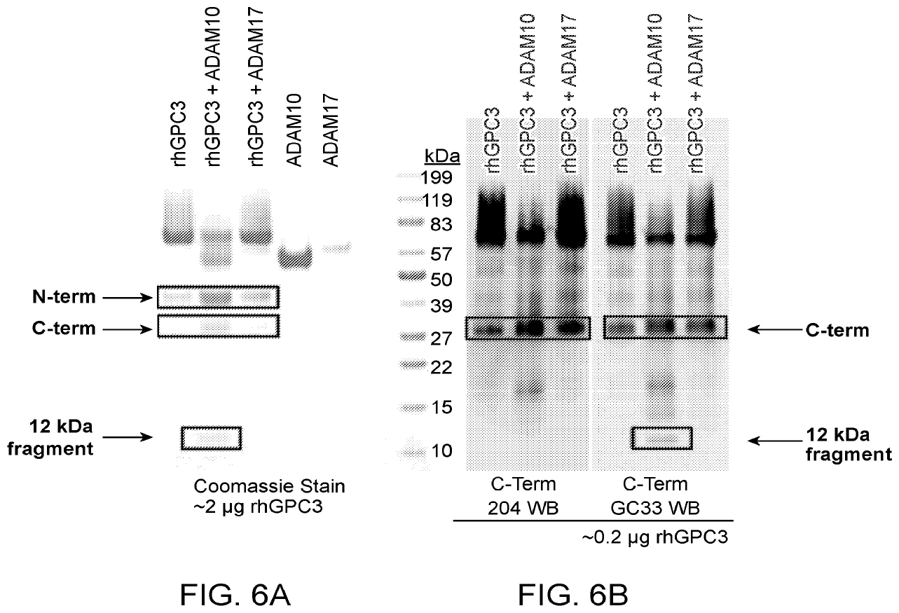

ADAM10 and ADAM17 cleavage of rhGPC3 was used to probe the binding region of 204. Depicted at FIG. 4 is a high-level illustration of the major GPC3 isoform (isoform 2), showing the furin cleavage site, possible 204 epitope, GC33 immunogen (524-562), 1G12 immunogen (511-580), and GC33 epitope (542-555). Depicted at FIG. 5 is a similar illustration, additionally showing a potential ADAM 10 cleavage site. ADAM10 and ADAM17 were used to cleave rhGPC3 (˜2 μg), samples were run on SDS-PAGE, and protein was visualized with coomassie stain (FIG. 6A). As shown, ADAM10 and ADAM17 (to a lesser degree) both appear to cleave rhGPC3 at the furin cleavage site, resulting in an increased intensity of the N-terminal and C-terminal fragments compared to undigested rhGPC3. Similarly prepared samples were analyzed via western blot where the primary antibody used was 204 and GC33 (FIG. 6B).

An ˜12 kDa ADAM 10 fragment (refer to FIGS. 6A-6B) is detected by the GC33 antibody and by coomassie, which corresponds to a predicted 11.7 kDa C-terminal GPC3AHS cleavage fragment between the proposed ADAM10 cleavage site and the GPI-anchor cleavage site (by notum). The 204 antibody does not detect this band, indicating that the epitope for the 204 antibody is somewhere between the furin-cleavage site and the predicted ADAM10 cleavage site.

### Example 3. Evaluation of Cell Surface GPC3 Via IHC

### Optimized GPC3 IHC Protocol for 204 mAb

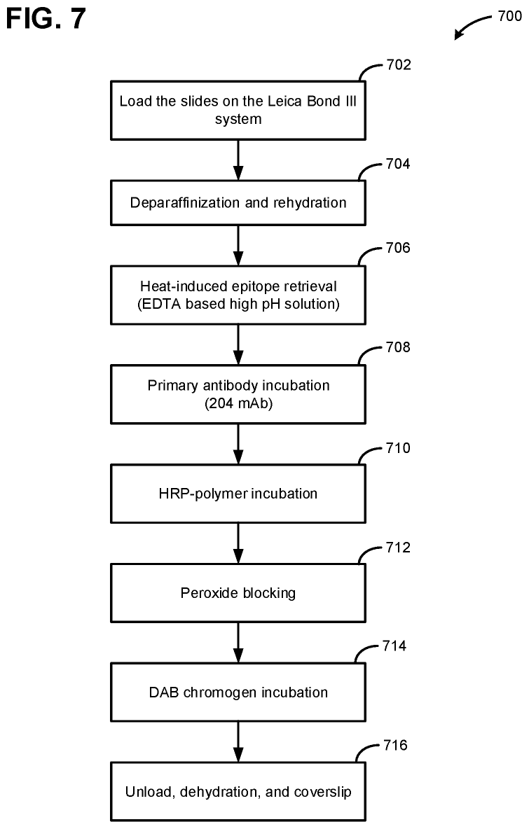

An overview of an optimized GPC3 IHC protocol for the 204 mAb is depicted as method 700 at FIG. 7. Briefly, step 702 of method 700 includes loading the slides on a Leica Bond Ill system (Leica Biosystems Inc., Richmond, IL) although other automated IHC staining systems may be used without departing from the scope of this disclosure. At step 704, method 700 includes deparrafinization and rehydration steps. At step 706, method 700 includes a heat-induced epitope retrieval step, which relies on an EDTA-based high pH solution. At step 708, method 700 includes incubating slides in the primary antibody (i.e., 204 mAb). Proceeding to step 708, method 700 includes incubation of slides with HRP-polymer-conjugated secondary antibody. Step 712 includes a peroxide blocking step, followed by incubation with DAB chromogen at step 714. Step 716 includes unloading, dehydration, and application of coverslip to the stained slides. While not explicitly illustrated, it may be understood that prior to conducting the methodology of FIG. 7, selected tissue was fixed and embedded in paraffin, followed by cutting of the tissue and mounting. Specifically, the protocol included 5/5/3 min dips in 95% EtOH, 3/3/3 min dips in 100% EtOH, followed by treatment with xylene (until clear/5/5 min dips), and then mounting. Deparaffinization at step 704 was performed using a standard protocol.

Thus, FIG. 7 represents a high-level methodology 700 corresponding to optimized GPC3 IHC staining protocol for the 204 mAb of the present disclosure. A more detailed version of the above-discussed optimized protocol is illustrated in Table 7, where steps are conducted in the order in which they descend in the table.

TABLE 7
Optimized membrane-bound GPC3 IHC staining procedure
for FFPE specimens on Leica Bond III staining system

| | Incubation | | | | |
|---|---|---|---|---|---|
| Step | time | | Dispense volume | Temperature | |
| Tissue sectioning | N/A | | N/A | Ambient | |
| and air drying | | | | | |
| Bake slides | 60 | min | N/A | 55-65° | C. |
| Load Slides onto | N/A | | N/A | Ambient | |
| Leica Bond III | | | | | |
| Bond Dewax Solution | N/A | | 150 μL, 3 times | 72° | C. |
| Alcohol | N/A | | 150 μL, 3 times | Ambient | |
| Bond Wash Solution | N/A | | 150 μL, 3 times | Ambient | |
| Bond ER Solution 2 | 20 | minutes | 150 μL, 4 times | 100° | C. |

| Bond Wash Solution | N/A | | 150 μL, 5 times | Ambient |
|---|---|---|---|---|
| Primary Antibody | 30 | minutes | 150 μL, 1 time | Ambient |
| (0.1 μg/mL; 1:5000 in | | | | |
| Bond Diluent) | | | | |
| Bond Wash Solution | N/A | | 150 μL, 3 times | Ambient |
| Post Primary | 8 | minutes | 150 μL, 1 time | Ambient |
| Bond Wash Solution | N/A | | 150 μL, 3 times | Ambient |
| Polymer | 8 | minutes | 150 μL, 1 time | Ambient |
| Bond Wash Solution | N/A | | 150 μL, 3 times | Ambient |
| Peroxide Block | 5 | minutes | 150 μL, 1 time | Ambient |
| Bond Wash Solution | N/A | | 150 μL, 3 times | Ambient |
| Deionized Water | N/A | | 150 μL, 2 times | Ambient |
| Mixed DAB Refine | 10 | minutes | 150 μL, 2 times | Ambient |
| Deionized Water | N/A | | 150 μL, 3 times | Ambient |
| Hematoxylin | 7 | minutes | 150 μL, 1 time | Ambient |

| | | | |
|---|---|---|---|
| Deionized Water | N/A | 150 μL, 3 times | Ambient |
| Bond Wash Solution | N/A | 150 μL, 1 time | Ambient |
| Deionized Water | N/A | 150 μL, 1 time | Ambient |
| Unload, Dehydrate, | N/A | N/A | Ambient |
| and coverslip | | | |

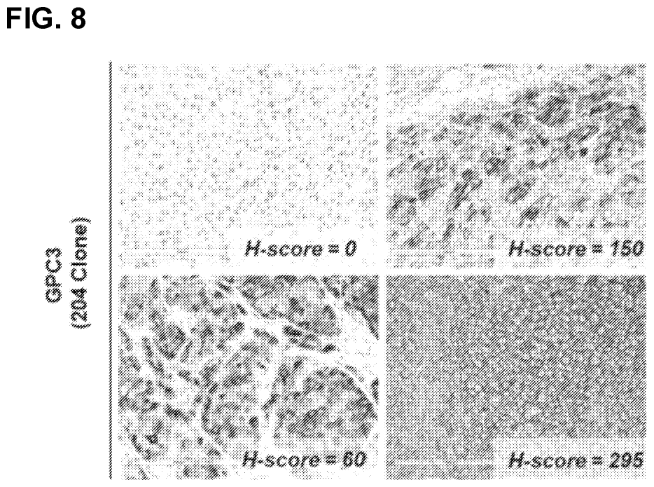

Exemplary results are depicted at FIG. 8. Specifically, FIG. 8 depicts representative images from a TMA of human HCC, where tissues were stained with the optimized protocol discussed above for 204 mAb. Staining was evaluated by a board-certified pathologist using the semi-quantitative membrane-bound GPC3 H-scoring as herein disclosed.

Comparison of 204 and 1G12 mAbs in Healthy Vs Diseased Lung and Liver Tissues

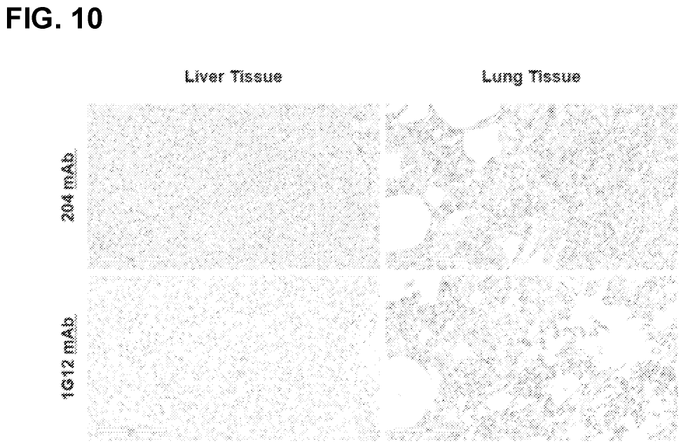

Using the optimized protocol discussed above for the 204 antibody, staining of healthy vs diseased tissue and corresponding membrane-associated H-scores was examined for the 204 antibody as compared to the 1G12 antibody. Specifically, the diseased tissue samples included squamous cell carcinoma of the lung, and HCC. Shown at FIG. 9 are representative examples of diseased tissue stained with 204 and 1G12 antibodies, along with corresponding membrane-associated H-score determinations. Shown at FIG. 10 are representative examples of the lack of staining observed with the 204 mAb as well as the 1G12 mAb in normal liver and lung tissues (i.e., adjacent tissues to the corresponding diseased tissue samples of FIG. 9).

Comparison of 1G12 and 204 in GPC3^{hi} and GPC3^{lo} Cells

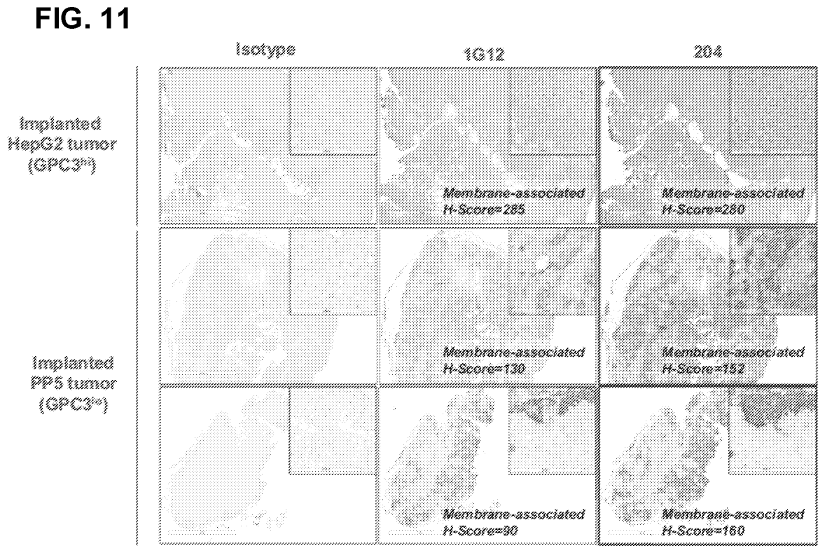

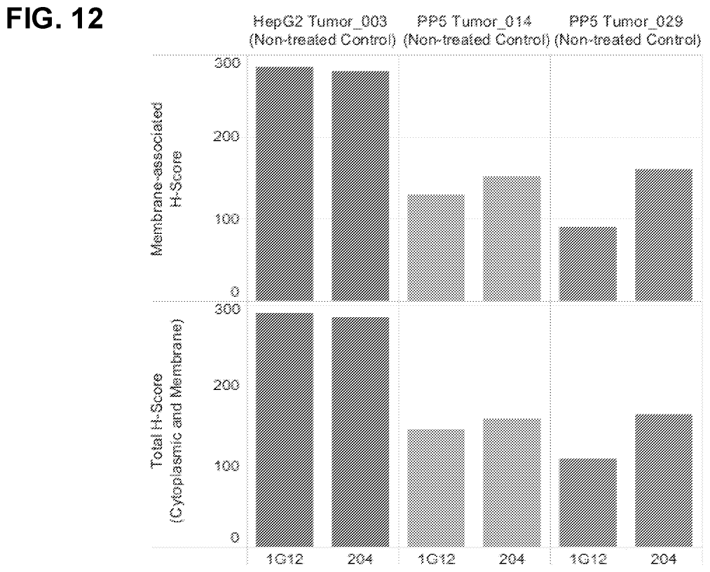

In this study, human HCC cell lines were implanted subcutaneously in NSG mice, and tumors were harvested on day 24 and day 31 post-implantation for PP5 and HepG2, respectively. PP5 tumors have considerably higher GPC3 expression variability as compared to HepG2 tumors, hence PP5 tumors have lower overall GPC3 expression. Thus, PP5 cells are herein referred to as GPC31° and HepG2 cells are herein referred to as GPC3hi. A similar protocol as that discussed above was used in this study. The IHC protocol used antibody titers selected by a pathologist, including 0.5 μg/mL for 1G12, and 0.1 μg/mL for 204. An isotype antibody was used as a negative control. As shown at FIG. 11, in this circumstance membrane-associated H-scores were substantially similar for the GPC3^{hi} cells. Alternatively, membrane-associated H-scores were significantly higher for GPC3^{lo} cells stained with the 204 antibody as compared to the 1G12 antibody. The data corresponding to FIG. 11 is quantified in FIG. 12.

### GPC3 Prevalence in Various Cancers

In this study, 204 and 1G12 antibodies were used to probe TMAs prepared from various types of cancer cells for GPC3 expression. Specifically, the types of TMA prepared for analysis with the 204 antibody included HCC, small cell carcinoma (SCC) of the lung, ovarian clear cell carcinoma (OCCC), and gastric cancer (stage III/IV). The types of TMA prepared for analysis with the 1G12 antibody included HCC, SCC, and OCCC. Two cores per subject were included in the HCC TMA in 28/29 subjects. With regard to the HCC TMA probed using the 204 antibody, membrane-associated H-scores from cores for all positive subjects were used to calculate the mean and the median. Data obtained using the 204 antibody is depicted in Table 8, and data obtained using the 1G12 antibody is depicted in Table 9.

TABLE 8
TMAs probed with 204 mAb

| | | | | | | Median of | Mean of |
|---|---|---|---|---|---|---|---|
| | | | Prevalence | | | mem.- | mem.- |
| | | | of cyto. | | Prevalence | associated | associated |
| | | Tot. # | plus | # mem.- | of mem.- | GPC3 H- | GPC3 H- |
| | Tot. # of | GPC3 | mem.- | associated | associated | score | score |
| Type of | cases | pos. | associated | GPC3 pos. | GPC3 pos. | from pos. | from pos. |
| TMA | evaluated | cases | GPC3 | cases | cases | cases only | cases only |
| HCC**, *** | 29 | 22 | 76% | 13 | 45% | 60.0 | 117 |
| SCC of the | 65 | 18 | 28% | 11 | 17% | 4.0 | 43.3 |
| Lung | | | | | | | |
| OCCC | 30 | 9 | 30% | 6 | 20% | 4.0 | 31.3 |
| Gastric | 120 | 3 | 3% | 1 | 1% | N/A | N/A |
| (III/IV)* | | | | | | | |

TABLE 9
TMAs probed with 1G12 mAb

| | | | | | | Median of | Mean of |
|---|---|---|---|---|---|---|---|
| | | | Prevalence | | | mem.- | mem.- |
| | | | of cyto. | | Prevalence | associated | associated |
| | | Tot. # | plus | # mem.- | of mem.- | GPC3 H- | GPC3 H- |
| | Tot. # of | GPC3 | mem.- | associated | associated | score | score |
| Type of | cases | pos. | associated | GPC3 pos. | GPC3 pos. | from pos. | from pos. |
| TMA | evaluated | cases | GPC3 | cases | cases | cases only | cases only |
| HCC** | 29 | 22 | 76% | 12 | 41% | 30.0 | 106.1 |
| SCC of the | 65 | 22 | 34% | 15 | 23% | 10.0 | 22.4 |
| Lung | | | | | | | |
| OCCC | 30 | 10 | 33% | 6 | 20% | 5.0 | 26.5 |

### Prevalence Distribution of Membrane-Associated GPC3

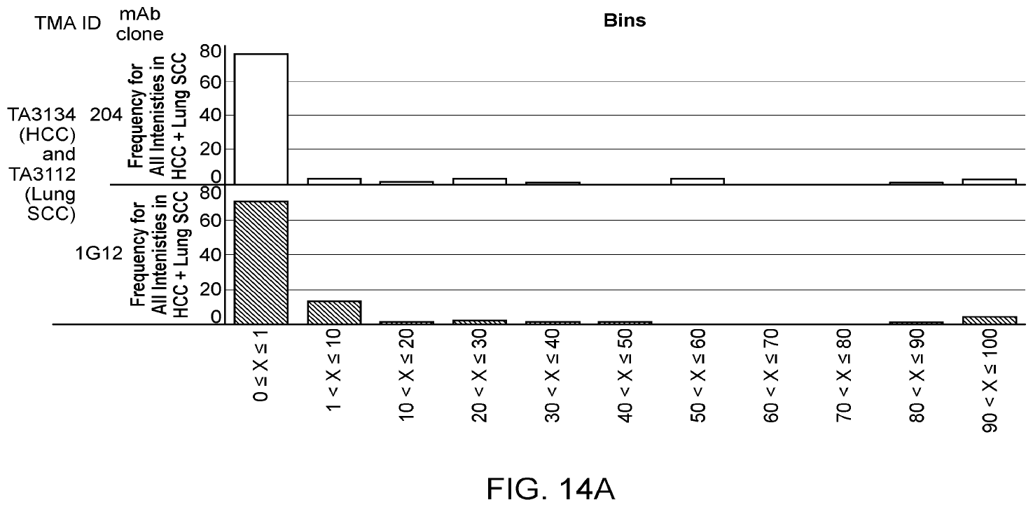

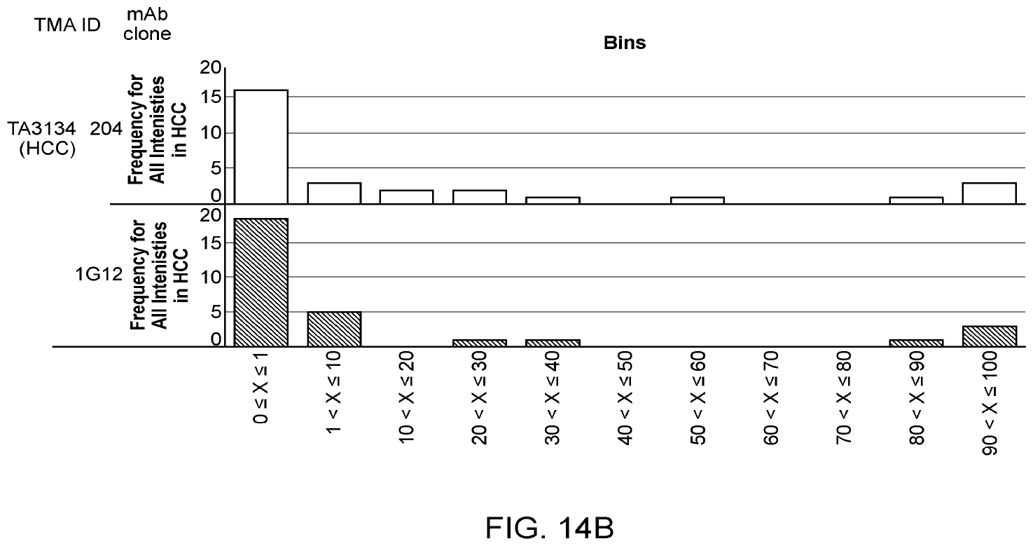

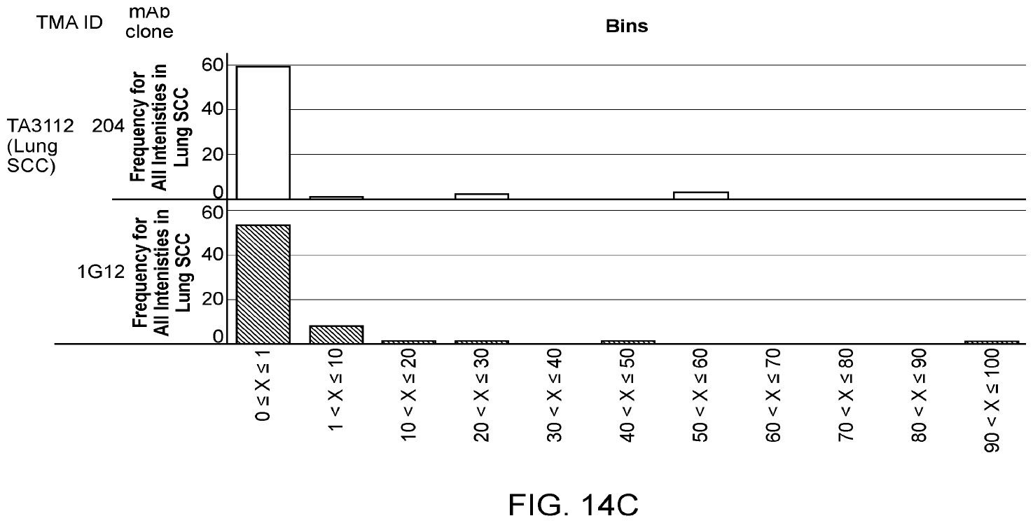

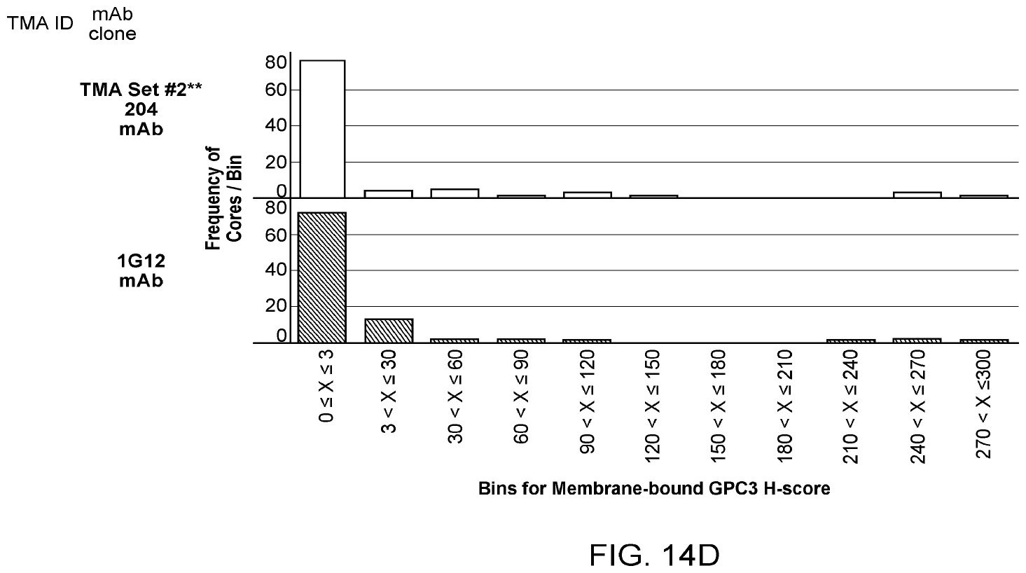

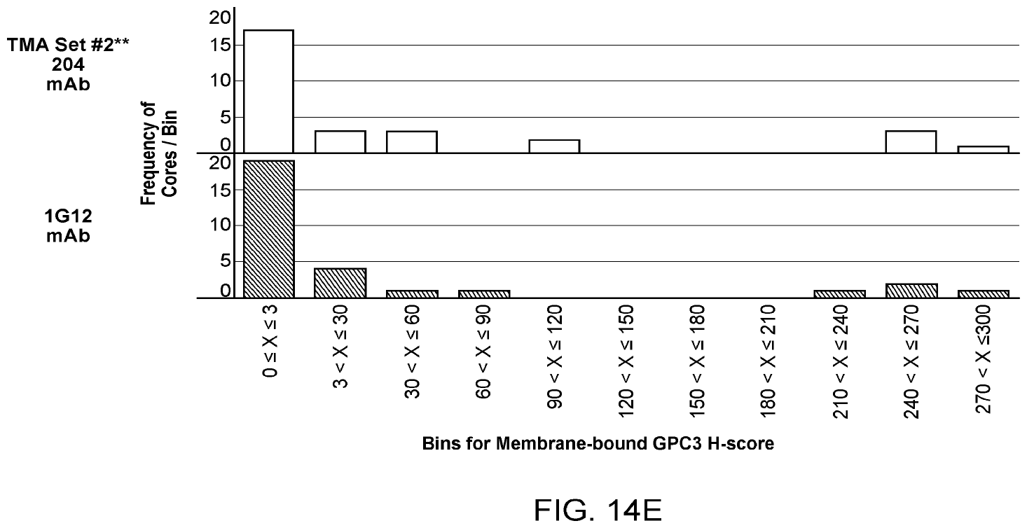

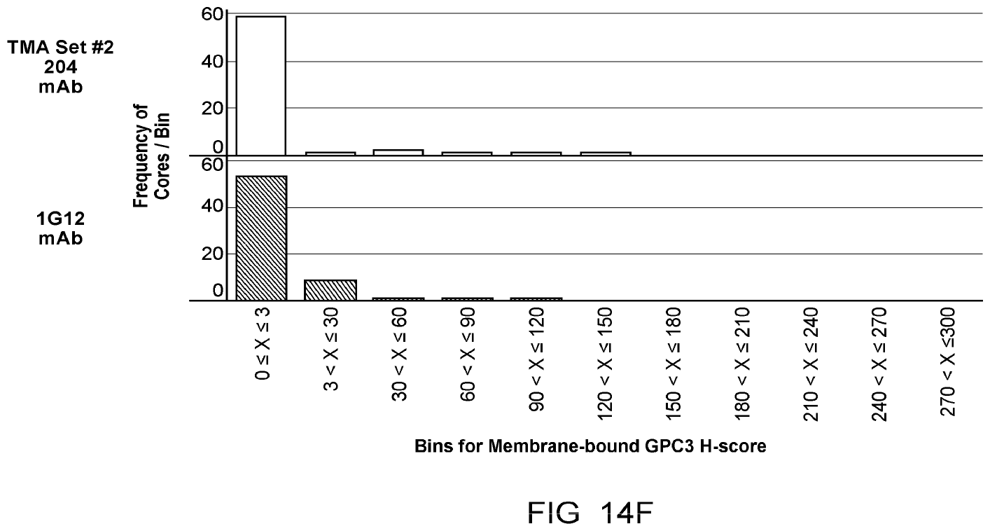

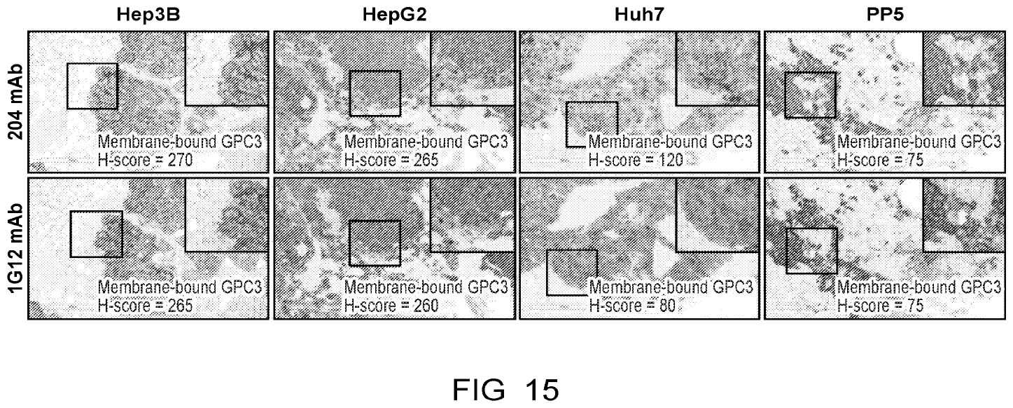

In this study, prevalence distribution of membrane-associated GPC3 in HCC and lung SCC (FIG. 14A), HCC (FIG. 14B), and lung SCC (FIG. 14C) was examined using each of the 204 and 1G12 antibodies. Staining intensity was scored using a semi-quantitative integer scale from 0 (negative) to 3 (or 3+) by a certified pathologist. Percent intensities were combined for 1+, 2+ and 3+. The first bin was set for 0-1%, the second bin was set at >1% to ≤10%, and so on in increments of 10%. With regard to FIGS. 14A and 14B, because two cores per subject are included in the HCC TMA TA3134 in most cases (28/29 subjects), intensities were averaged in those cases.

Prevalence distribution of membrane-associated GPC3 in HCC and lung SCC (FIG. 14D), HCC (FIG. 14E), and lung SCC (FIG. 14E) was also examined using membrane-specific H-scores obtained from IHC staining using the 204 and 1G12 antibodies. Membrane-bound GPC3 prevalence distribution was evaluated by assessing membrane-specific H-scores starting at ≤3 (≤1%), >3 (>1%) and ≤30 (≤10%), and then increments of 30 (10%). Similar to that discussed with regard to FIGS. 14A-14C, two cores per subject (sample) were included in the HCC TMA in 28/29 subjects for the analysis using the 204 and 1G12 mAbs. To calculate the mean and median of membrane-bound H-scores, cores from all positive subjects were used.

### GPC3 Prevalence in Cirrhotic and HCC Patients

In this study, membrane-associated GPC3 presence in cirrhotic and HCC patients was evaluated using the 204 antibody as compared to the 1G12 antibody. The types of TMA analyzed included tissue from HCC patients, adjacent normal liver tissue (with mild inflammation), and liver tissues from patients with cirrhosis alone/plus inflammation or hepatitis. Two cores per subject were included in the HCC TMA in 28/29 subjects. Data obtained using the 204 antibody is depicted in Table 10, and data obtained using the 1G12 antibody is depicted in Table 11. As illustrated, GPC3 expression was not detected on the cell membrane of hepatocytes in patients with cirrhosis when probed using the 204 antibody or the 1G12 antibody.

TABLE 10
GPC3 prevalence in cirrhotic and HCC patients assessed in TMAs probed with 204 mAb

| | | | Prevalence of | | Prevalence of |
|---|---|---|---|---|---|
| | | | cyto. plus | # mem.- | mem.- |
| | Tot. # of | | mem.- | associated | associated |
| | cases | Tot. # GPC3 | associated | GPC3 pos. | GPC3 pos. |
| Type of TMA | evaluated | pos. cases | GPC3 | cases | cases |
| Adjacent normal liver tissue (mild inflammation) | 26 | 0 | 0% | 0 | 0% |
| Liver tissues from patients with Cirrhosis | 35 | 2 | 6% | 0 | 0% |
| alone/plus inflammation or hepatitis | | | | | |
| HCC | 29 | 22 | 76% | 13 | 45% |

TABLE 11
GPC3 prevalence in cirrhotic and HCC patients assessed in TMAs probed with 1G12 mAb

| | | | Prevalence of | | Prevalence of |
|---|---|---|---|---|---|
| | | | cyto. plus | # mem.- | mem.- |
| | Tot. # of | | mem.- | associated | associated |
| | cases | Tot. # GPC3 | associated | GPC3 pos. | GPC3 pos. |
| Type of TMA | evaluated | pos. cases | GPC3 | cases | cases |
| Adjacent normal liver tissue (mild inflammation) | 27 | 0 | 0% | 0 | 0% |
| Liver tissues from patients with Cirrhosis | 37 | 2 | 5% | 0 | 0% |
| alone/plus inflammation or hepatitis | | | | | |
| HCC | 29 | 22 | 76% | 12 | 41% |

### Performance Characteristics: 204 Versus 1G12

Performance characteristics of the 204 antibody were examined. Tissues from formalin-fixed paraffin-embedded (FFPE) blocks and TMAs in the analysis included normal adjacent tissue (NAT), liver cirrhosis tissue, HCC, SCC of the lung, and OCCC. Sensitivity was calculated as the number of true positive assessment divided by number of all positive assessment (TP/(TP+FN)) (TP: true positive; FN: false negative). Specificity was calculated as the number of true negative assessment divided by the number of all negative assessment (TN/(TP+FP)) (TN: true negative; FP: false positive). Accuracy was calculated as the number of correct assessments divided by the number of all assessments ((TN+TP)/(TN+TP+FN+FP)). To be acceptable for proceeding with Laboratory Developed Test (LDT) validation, all three parameters (accuracy, sensitivity, and specificity) had to have a >90% concordance. Results are shown at Table 12.

TABLE 12
204 performance characteristics -
Combined FFPE blocks and TMAs

| | Concordance | Concordance | Concordance |
|---|---|---|---|
| | (cutoff >1%) | (cutoff >5%) | (cutoff >10%) |
| True positive | 42 | 32 | 28 |
| True negative | 207 | 217 | 221 |
| False positive | 4 | 9 | 10 |
| False negative | 7 | 2 | 1 |
| Accuracy | 96% | 96% | 96% |
| Sensitivity | 86% | 94% | 97% |
| Specificity | 98% | 96% | 96% |

Table 13 depicts additional data restricted to just FFPE NAT and tumor blocks, and Table 14 depicts additional data restricted to FFPE NAT, liver cirrhosis and tumor cores from TMAs. Again taken together, the results demonstrate acceptable accuracy sensitivity, and specificity to proceed with LDT validation for 204 using a temporary cutoff of >5% or >10%.

TABLE 13
204 performance characteristics - FFPE NAT and tumor blocks

| | Concordance | Concordance | Concordance |
|---|---|---|---|
| | (Cutoff >1%) | (Cutoff >5%) | (Cutoff >10%) |
| True positive | 13 | 11 | 11 |
| True negative | 23 | 24 | 24 |
| False positive | 1 | 2 | 2 |
| False negative | 0 | 0 | 0 |
| Accuracy | 97% | 95% | 95% |
| Sensitivity | 100% | 100% | 100% |
| Specificity | 96% | 92% | 92% |

TABLE 14
204 performance characteristics - FFPE NAT,
liver cirrhosis and tumor cores from TMAs

| | Concordance | Concordance | Concordance |
|---|---|---|---|
| | (Cutoff >1%) | (Cutoff >5%) | (Cutoff >10%) |
| True positive | 29 | 21 | 17 |
| True negative | 184 | 193 | 197 |
| False positive | 3 | 7 | 8 |
| False negative | 7 | 2 | 1 |
| Accuracy | 96% | 96% | 96% |
| Sensitivity | 81% | 91% | 94% |
| Specificity | 98% | 97% | 96% |

### Head-to-Head Comparison of 204 vs 1G12

In this study, FFPE tumor blocks and FFPE tumor cores were probed with 204 or 1G12 antibody, and membrane-associated H-scores were calculated. The source of the tumor blocks and tumor cores, along with # of tumor blocks/# of tumor cores for each tissue source, and # of cases, is depicted at Table 15.

TABLE 15
Tumor blocks, tumor cores, and number of cases relied upon
for head-to-head comparison of 204 and 1G12 antibodies.

| | | Tumor | # of |
|---|---|---|---|
| | Tumor | Cores | cases |
| Indications | Blocks | (TMA) | (TMA) |
| Gastric cancer, Adenocarcinoma* | 6 | N/A | N/A |
| Liver Cancer, HCC** | 8 | 57 | 29 |
| Lung Cancer, SCC | 9 | 65 | 65 |
| Ovarian Cancer, CCC | 9 | 30 | 30 |

In the analysis, TMA for gastric cancer using 1G12 IHC staining was not included in the head-to-head comparison. With regard to the HCC samples, two cores per subject were included in the HCC TMA in more cases. The amount of tissue available is the key difference between FFPE tumor blocks and cores, otherwise the samples were processed in a similar manner.

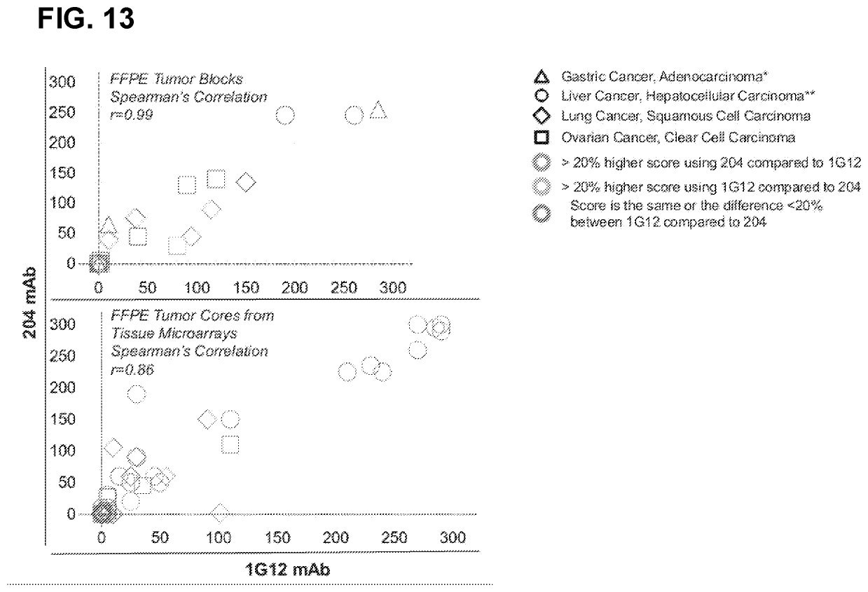

FIG. 13 depicts membrane-associated H-scores obtained using the 204 antibody plotted against membrane-associated H-scores obtained using the 1G12 antibody for both FFPE tumor blocks (top, n=32 samples) and FFPE tumor cores from TMAs (bottom, n=152 samples). As shown, membrane-associated H-scores obtained using the 204 antibody are in good concordance with those using the 1G12 antibody (Spearman's correlation of r=0.99 for the FFPE tumor blocks and r=0.86 for FFPE tumor cores from TMAs). Furthermore, IHC staining using the 204 antibody resulted in higher membrane-associated H-scores in tumor blocks, and particularly tumor cores, more frequently relative to 1G12 staining on the same TMA.

Membrane-Associated GPC3 Expression in FFPE Tissues from Xenograft Tumor Models

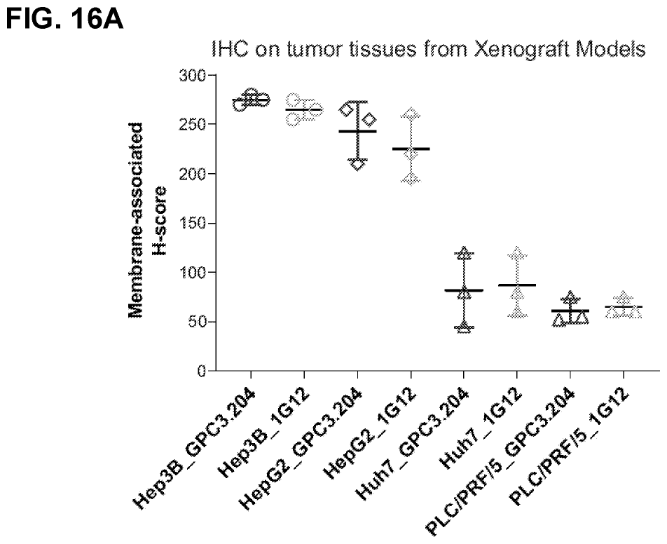

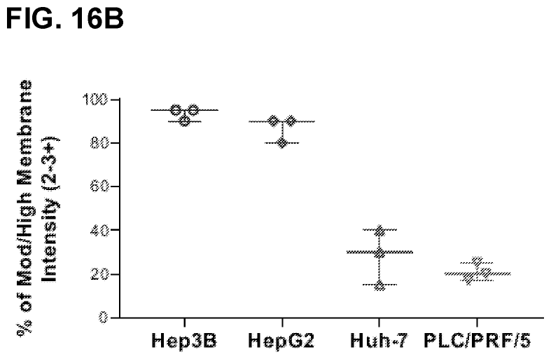

In this study, the 204 antibody was used to probe GPC3 expression in various FFPE tissues from xenograft tumor models. Specifically, the tumor models included Hep3B, HepG2, Huh-7, and PLC/PRF/5 (which are all immortalized cell lines of human HCC). IHC staining was conducted with 204 or 1G12 antibodies to illustrate differences in staining and corresponding membrane-associated H-score for the different antibodies. Briefly, tumor cells were mixed 1:1 with Matrigel and PBS, and then the cells were implanted subcutaneously via the right hind flank into ICR-SCID mice. Images at FIG. 15 are representative of GPC3 IHC staining using the 204 or 1G12 mAbs. The larger square in each image represents a higher resolution image of the region comprising the smaller square for each image. Further illustrated are corresponding calculated membrane-associated H-scores, illustrating equivalent or higher membrane-associated H-scores obtained when using 204 mAb as compared to 1G12 mAb. FIG. 16A shows a plot of the determined membrane-associated H-scores obtained using the 204 or 1G12 antibodies, and FIG. 16B illustrates a plot of calculated % of moderate/high membrane intensity corresponding to just the 204 antibody.

### Comparison of Membranous Staining Vs Cytoplasmic Staining for 204 and 1G12 Antibodies

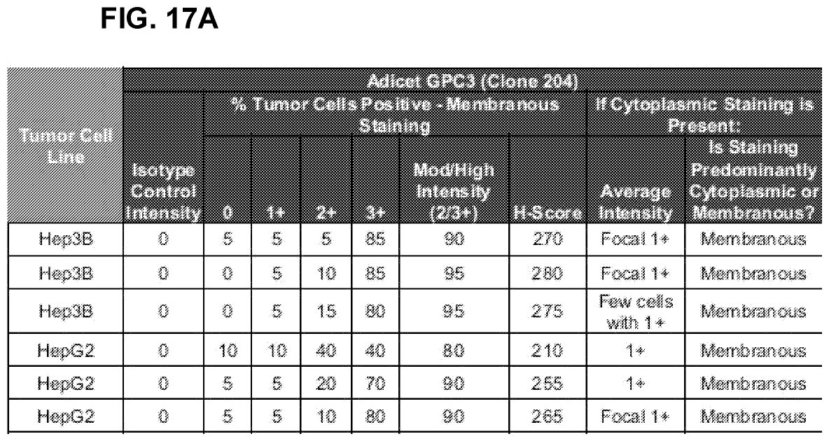

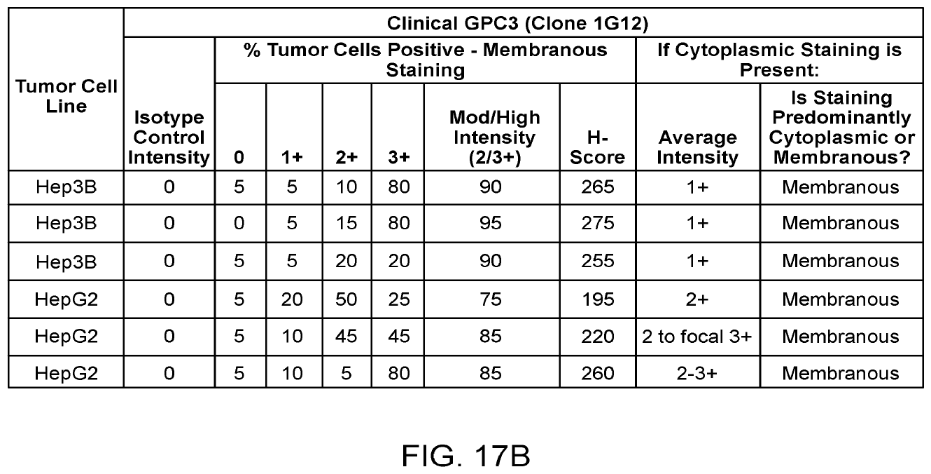

In this study, tissue from Hep3B and HepG2 xenografts were subjected to IHC analysis using the 204 or 1G12 antibody, and intensities and H-scores corresponding to membranous staining was determined. Further, average intensity corresponding to cytoplasmic staining was determined, and it was assessed as to whether the staining was primarily cytoplasmic or membranous. The results obtained using the 204 antibody are depicted at FIG. 17A, and the results obtained using the 1G12 antibody are depicted at FIG. 17B. As shown, with both the 204 and 1G12 antibodies, staining was predominantly membranous for the tumor cell lines Hep3B and HepG2. However, membrane-associated H-scores were generally higher for 204, and average intensity of cytoplasmic staining was somewhat lesser with the 204 antibody as compared with the 1G12 antibody.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. An isolated monoclonal antibody that binds glypican-3 (GPC3), wherein:
the heavy chain of the anti-GPC3 antibody comprises a complementary determining region (CDR) 1 set forth as SEQ ID NO: 6, a CDR2 set forth as SEQ ID NO: 8, and a CDR3 set forth as SEQ ID NO: 10, and the light chain of the antibody comprises a CDR1 set forth as SEQ ID NO: 13, a CDR2 set forth as SEQ ID NO: 15, and a CDR3 set forth as SEQ ID NO: 17.

2. The isolated monoclonal antibody of claim 1, wherein:
The heavy chain of the antibody comprises a CDR1, a CDR2, and a CDR3, respectively set forth as amino acid residues 31-35, 50-66, and 99-105 of SEQ ID NO: 2, and the light chain of the antibody comprises a CDR1, a CDR2, and a CDR3 respectively set forth as amino acid residues 24-34, 50-56, and 89-97 of SEQ ID NO: 4.

3. The isolated monoclonal antibody of claim 1, wherein:
the heavy chain of the antibody comprises SEQ ID NO: 2, and the light chain of the antibody comprises SEQ ID NO: 4; or
the antibody comprises:
(i) a variable heavy (VH) domain comprising the amino acid sequence of SEQ ID NO: 2; and
(ii) a variable light (VL) domain comprising the amino acid sequence of SEQ ID NO: 4.

4. (canceled)

5. The isolated monoclonal antibody of claim 1, wherein:
the antibody is a chimeric, humanized, or human antibody;
the antibody is a bispecific antibody; or
the antibody is an antibody fragment.

6. (canceled)

7. (canceled)

8. The isolated monoclonal antibody of claim 5, wherein the antibody is a Fab fragment, a Fab′ fragment, a F(ab)′₂ fragment, a single chain variable fragment (scFv), or a disulfide stabilized variable fragment (dsFv).

9. A method of detecting GPC3 in a tissue preparation, the method comprising:
contacting the tissue preparation with the isolated monoclonal antibody of claim 1 under conditions sufficient for formation of a complex of the isolated monoclonal antibody of claim 1 with GPC3 present on the cell membrane of cells of the tissue preparation; and
detecting binding of the antibody to the tissue preparation.

10. The method of claim 9, wherein the tissue preparation comprises a hepatocellular carcinoma (HCC), melanoma, squamous cell carcinoma of the lung, Merkel cell carcinoma, or ovarian clear cell carcinoma tumor biopsy.

11. The method of claim 9, wherein the monoclonal antibody is directly labeled.

12. The method of claim 9, further comprising:
contacting a second antibody that specifically binds the monoclonal antibody with the tissue preparation; and
detecting the binding of the second antibody.

13. The method of claim 9, wherein detecting the binding of the antibody to the tissue sample further comprises scoring an amount of the complex detected.

14. The method of claim 13, wherein said scoring is done by a pathologist.

15. The method of claim 13, wherein said detecting the presence of the complex is done via digitization; and
wherein said scoring is automated based on the digitization of the detected complex.

16. The method of claim 13, wherein said scoring further comprises determining a staining intensity of the complex detected via immunohistochemistry using an integer scale from 0 (negative) to 3+, recording the percentage of positively stained cells at each intensity level, and calculating a membrane-associated H-score based on the percentage of positively stained cells at each intensity level.

17. A method for predicting a therapeutic effect of an anti-GPC3 immunotherapy on a cancer, the cancer characterized in that cells of the cancer express GPC3, the method comprising:
detecting the presence of said cells in a subject via the method of claim 9, wherein when the complex of the anti-GPC3 antibody with GPC3 expressed on the membrane of the cancer cells is detected, the anti-GPC3 immunotherapy is predicted to have a therapeutic effect on the cancer in the subject.

18. The method of claim 17, wherein the method of predicting the therapeutic effect is conducted prior to the subject having received any anti-GPC3 immunotherapy.

19. The method of claim 17, wherein the method of predicting the therapeutic effect is conducted while the subject is already in the process of receiving the anti-GPC3 immunotherapy.

20. The method of claim 17, wherein the anti-GPC3 immunotherapy comprises an anti-GPC3 antibody, or a chimeric antigen receptor (CAR) T cell therapy, or CAR NK cell therapy, wherein the CAR is designed to specifically recognize membrane-bound GPC3.

21. (canceled)

22. An isolated nucleic acid molecule encoding the monoclonal antibody of claim 1.

23. The isolated nucleic acid molecule of claim 22, wherein:
a nucleotide sequence encoding the heavy chain of the monoclonal antibody comprises SEQ ID NO: 1 and a nucleotide sequence encoding the light chain of the antibody comprises SEQ ID NO: 3.

24. An expression vector comprising the isolated nucleic acid molecule of claim 22, or a host cell transformed with the expression vector.

25. (canceled)

26. (canceled)

27. An antibody-drug conjugate (ADC), comprising the isolated monoclonal antibody of claim 1.

28. A chimeric antigen receptor (CAR) comprising the antibody fragment of claim 7.

29. A modified immune cell, comprising a chimeric antigen receptor (CAR), wherein said CAR comprises the CAR of claim 28, or a plurality of the modified immune cells.

30. The modified immune cell of claim 29, wherein the modified immune cell is a modified T cell or a modified NK cell.

31. The modified immune cell of claim 30, wherein the modified immune cell is an αβ T cell, or a γδ T cell.

32. (canceled)

33. (canceled)

34. (canceled)

35. A method of inhibiting the growth of a cell that displays a GPC3 epitope that is specifically recognized by the antibody of claim 1, comprising contacting said cell with the isolated monoclonal antibody of claim 1.

36. A composition comprising a therapeutically effective amount of the isolated monoclonal antibody of claim 1, and a pharmaceutically acceptable carrier.

37. A method of treating a subject having a cancer, comprising selecting a subject with a cancer that expresses GPC3 and administering to said subject the composition according to claim 35, thereby treating the cancer in the subject.

38. The method of claim 37, wherein the cancer is liver cancer, ovarian cancer, gastric cancer, Merkel cell carcinoma, or lung cancer.

39. (canceled)

40. (canceled)

41. (canceled)

42. (canceled)


SEQUENCE LISTING (18 sequences)

SEQ ID NO: 1 - DNA, 348 bp, synthetic construct
  source 1..348: mol_type = other DNA
gaggtccagctgcaacagtctggacctgagctggtgaagcctggggcttcagtgaagatatcctgcaagacttctggatacacattcactgaatacgccatgcactgggtgaagcagagccatggaaagagccttgagtggattggaggtattaatcctaacaatggtgttactacttacaaccagaggttcaagggcaaggccacattgactgtagacaagtcctccagcacagcctacatggagctccgcagcctgacatctgaggattctgcagtctattactgtgcaagaggcctactatggtatgcttactggggccaagggactctggtcactgtctctgca

SEQ ID NO: 2 - protein, 116 aa, synthetic construct
EVQLQQSGPELVKPGASVKISCKTSGYTFTEYAMHWVKQSHGKSLEWIGGINPNNGVTTYNQRFKGKATLTVDKSSSTAYMELRSLTSEDSAVYYCARGLLWYAYWGQGTLVTVSA

SEQ ID NO: 3 - DNA, 321 bp, synthetic construct
  source 1..321: mol_type = other DNA
gacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttcctctcacgttcggtgctgggaccaagctggagctgaaa

SEQ ID NO: 4 - protein, 107 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDEFPLTFGAGTKLELK

SEQ ID NO: 5 - protein, 30 aa, synthetic construct
EVQLQQSGPELVKPGASVKISCKTSGYTFT

SEQ ID NO: 6 - protein, 5 aa, synthetic construct
EYAMH

SEQ ID NO: 7 - protein, 14 aa, synthetic construct
WVKQSHGKSLEWIG

SEQ ID NO: 8 - protein, 17 aa, synthetic construct
GINPNNGVTTYNQRFKG

SEQ ID NO: 9 - protein, 32 aa, synthetic construct
KATLTVDKSSSTAYMELRSLTSEDSAVYYCAR

SEQ ID NO: 10 - protein, 7 aa, synthetic construct
GLLWYAY

SEQ ID NO: 11 - protein, 11 aa, synthetic construct
WGQGTLVTVSA

SEQ ID NO: 12 - protein, 23 aa, synthetic construct
DIKMTQSPSSMYASLGERVTITC

SEQ ID NO: 13 - protein, 11 aa, synthetic construct
KASQDINSYLS

SEQ ID NO: 14 - protein, 15 aa, synthetic construct
WFQQKPGKSPKTLIY

SEQ ID NO: 15 - protein, 7 aa, synthetic construct
RANRLVD

SEQ ID NO: 16 - protein, 32 aa, synthetic construct
GVPSRFSGSGSGQDYSLTISSLEYEDMGIYYC

SEQ ID NO: 17 - protein, 9 aa, synthetic construct
LQYDEFPLT

SEQ ID NO: 18 - protein, 10 aa, synthetic construct
FGAGTKLELK